(19) United States
(12) Patent Application Publication
(10) Pub. No.: US 2022/0282398 A1
(43) Pub. Date: Sep. 8, 2022

(54) UNIVERSAL BLOCKING OLIGONUCLEOTIDES FOR REDUCED OFF-TARGET HYBRIDIZATION IN HYBRIDIZATION CAPTURE METHODS

(71) Applicant: BIOO Scientific Corporation, Austin, TX (US)

(72) Inventor: Kerry Gunning, Austin, TX (US)

(21) Appl. No.: 17/688,062

(22) Filed: Mar. 7, 2022

Related U.S. Application Data
(60) Provisional application No. 63/157,082, filed on Mar. 5, 2021.

(51) Int. Cl. C40B 30/04; C12Q 1/6806; C12Q 1/6853; C12Q 1/6832; C40B 40/08

(52) U.S. Cl. CPC C40B 30/04; C12Q 1/6806; C12Q 1/6853; C12Q 1/6832; C40B 40/08

(57) ABSTRACT

Sets of hybridization blockers, kits include at least one set of hybridization blockers, and methods of use thereof in massively parallel nucleic acid sequencing, are provided according to aspects of the present disclosure, each of the hybridization blockers comprising at least one Tm increasing nucleotide, the set of hybridization blockers for use in massively parallel sequencing of a plurality of nucleic acid sequencing library molecules, wherein the set of hybridization blockers efficiently blocks the complementary strand interactions between the adapter regions of different library molecules and is therefore effective to reduce the capture of non-target sequences during a capture enrichment hybridization to maximize the efficiency of the massively parallel sequencing techniques.

### REFERENCE TO RELATED APPLICATION

This application claims priority to U.S. Provisional Patent Application Ser. No. 63/157,082, filed Mar. 5, 2021, the entire content of which is incorporated herein by reference.

### FIELD OF THE INVENTION

According to general aspects of the present disclosure, compositions and methods are provided for reducing hybridization of non-target nucleic acids in sequencing libraries for massively parallel sequencing. According to specific aspects of the present disclosure, sets of hybridization blockers are provided for reducing hybridization of non-target nucleic acids in sequencing libraries for massively parallel sequencing.

### BACKGROUND OF THE INVENTION

Genetic analysis has become increasingly common and is useful in a wide variety of molecular biology applications. For example, genetic testing of individuals is particularly useful for early detection of genetic diseases and can play a role in selection of treatments for a particular disease or condition.

Massively parallel nucleic acid sequencing techniques play a key role in genetic analysis of target nucleic acids but produce less than optimal results when non-target nucleic acids are sequenced along with target nucleic acids.

Methods of target enrichment are often used to reduce contamination of sequencing results with non-target sequences in massively parallel nucleic acid sequencing techniques. One such enrichment method relies on hybridization capture of target nucleic acid sequencing library molecules.

However, sequencing libraries for massively parallel nucleic acid sequencing include adapter sequences attached to insert sequences in nucleic acid sequencing library molecules. Adapters include nucleic acid sequences with various functional properties, such as extension primer-binding sites, one or more index sequences for sample identification, sequencing primer-binding sites, and amplification primer-binding sites. Thus at least some portions of the adapters are identical and present on all nucleic acid sequencing library molecules produced for use in a given massively parallel nucleic acid sequencing technique. Further, sequencing library molecules include two complementary strands, including complementary adapters, such that hybridization steps of massively parallel nucleic acid sequencing techniques can result in “daisy chaining” of adapter sequences and these can be retained by hybridization capture during enrichment steps, thereby capturing non-target nucleic acids and wasting reagents and time sequencing the non-target nucleic acids.

The expanding applications of massively parallel sequencing techniques and increasing need to multiplex larger numbers of individual libraries on a single run has resulted in the need to more efficiently block the complementary strand interactions between the adapter regions of different library molecules to reduce the capture of non-target sequences during a capture enrichment hybridization to maximize the efficiency of the massively parallel sequencing techniques. There is a continuing need for compositions and methods for reducing hybridization of non-target nucleic acids in sequencing libraries for massively parallel sequencing.

### SUMMARY OF THE INVENTION

Sets of hybridization blockers, kits including at least one set of hybridization blockers, and methods of use thereof, are provided according to aspects of the present disclosure, each of the hybridization blockers comprising at least one Tm increasing nucleotide, the set of hybridization blockers for use in massively parallel sequencing of a plurality of nucleic acid sequencing library molecules, wherein the set of hybridization blockers efficiently blocks the complementary strand interactions between the adapter regions of different library molecules and is therefore effective to reduce the capture of non-target sequences during a capture enrichment hybridization to maximize the efficiency of the massively parallel sequencing techniques.

Sets of hybridization blockers, and kits including them, for use in massively parallel sequencing of a plurality of nucleic acid sequencing library molecules according to aspects of the present disclosure are provided.

For reference in description of particular kits, hybridization blockers, and methods, it will be understood that the plurality of nucleic acid sequencing library molecules to be sequenced each has a template strand and a complementary strand, the template strand comprising an insert disposed between a first adapter and a second adapter, the complementary strand comprising a complement of the insert disposed between a complement of the first adapter and a complement of the second adapter.

In some instances, the first adapter comprises a first extension primer-binding site and/or a first sequencing primer-binding site, and/or the second adapter comprises a second extension primer-binding site and/or a second sequencing primer-binding site, the complement of the first adapter comprises a corresponding complement of the first extension primer-binding site and/or a corresponding complement of the first sequencing primer-binding site, and/or the complement of the second adapter comprises a corresponding complement of the second extension primer-binding site and/or a corresponding complement of the second sequencing primer-binding site.

In some instances, the insert of the template strand is a transposon insert disposed between a first transposon end and a second transposon end, the first transposon end is adjacent to the first sequencing primer-binding site and the second transposon end is adjacent to the second sequencing primer-binding site, and the complementary strand comprises a complement of the transposon insert, the first transposon end and the second transposon end.

Sets of hybridization blockers, and kits including them, for use in massively parallel sequencing of a plurality of nucleic acid sequencing library molecules according to aspects of the present disclosure is provided wherein each hybridization blocker comprises an oligonucleotide having a nucleic acid sequence which is a complement of an oligonucleotide present in the template strand and/or complementary strand of at least some of the plurality of nucleic acid sequencing library molecules, with the proviso that none of the hybridization blockers comprises an oligonucleotide having a nucleic acid sequence which is a complement of the insert sequence, or an index sequence, if present, of the nucleic acid sequencing library molecules. According to aspects of the present disclosure, each oligonucleotide of each of the nine hybridization blockers comprises a plurality of Tₘ increasing nucleotide analogs.

Sets of hybridization blockers, and kits including them, for use in massively parallel sequencing of a plurality of nucleic acid sequencing library molecules according to aspects of the present disclosure comprises at least two pairs of split hybridization blockers, each pair of split hybridization blockers comprises a first split oligonucleotide having an oligonucleotide sequence complementary to a portion A of a target oligonucleotide included in the template strand or the complementary strand and a second split oligonucleotide having an oligonucleotide sequence complementary to a portion B of the target oligonucleotide included in the template strand or the complementary strand, wherein portion A and portion B are adjacent in the target oligonucleotide, and wherein the portion A and the portion B of the target oligonucleotide to which each pair of split hybridization blockers hybridizes, is different.

Sets of hybridization blockers, and kits including them, for use in massively parallel sequencing of a plurality of nucleic acid sequencing library molecules according to aspects of the present disclosure comprises at least two pairs of split hybridization blockers, wherein the first pair of split hybridization blockers comprises a first split oligonucleotide having an oligonucleotide sequence complementary to a portion A of the first sequencing primer-binding site and a second split oligonucleotide having an oligonucleotide sequence complementary to a portion B of the first sequencing primer-binding site, or wherein the first pair of split hybridization blockers comprises a first split oligonucleotide having an oligonucleotide sequence complementary to a portion A of the complement of the first sequencing primer-binding site and a second split oligonucleotide having an oligonucleotide sequence complementary to a portion B of the complement of the first sequencing primer-binding site.

Sets of hybridization blockers, and kits including them, for use in massively parallel sequencing of a plurality of nucleic acid sequencing library molecules according to aspects of the present disclosure comprises at least two pairs of split hybridization blockers, wherein the second pair of split hybridization blockers comprises a first split oligonucleotide having an oligonucleotide sequence complementary to a portion A of the second sequencing primer-binding site and a second split oligonucleotide having an oligonucleotide sequence complementary to a portion B of the second sequencing primer-binding site, or wherein the second pair of split hybridization blockers comprises a first split oligonucleotide having an oligonucleotide sequence complementary to a portion A of the complement of the second sequencing primer-binding site and a second split oligonucleotide having an oligonucleotide sequence complementary to a portion B of the complement of the second sequencing primer-binding site.

Sets of hybridization blockers, and kits including them, for use in massively parallel sequencing of a plurality of nucleic acid sequencing library molecules according to aspects of the present disclosure comprises a first pair of split hybridization blockers comprising: a first split oligonucleotide having an oligonucleotide sequence complementary to at least a portion of the first sequencing primer-binding site; and a second split oligonucleotide having an oligonucleotide sequence complementary to at least a portion of the first transposon end.

Sets of hybridization blockers, and kits including them, for use in massively parallel sequencing of a plurality of nucleic acid sequencing library molecules according to aspects of the present disclosure comprises a first pair of split hybridization blockers comprising: a first split oligonucleotide having an oligonucleotide sequence complementary to at least a portion of the complement of the first sequencing primer-binding site; and a second split oligonucleotide having an oligonucleotide sequence complementary to at least a portion of the complement of the first transposon end.

Sets of hybridization blockers, and kits including them, for use in massively parallel sequencing of a plurality of nucleic acid sequencing library molecules according to aspects of the present disclosure comprises a second pair of split hybridization blockers comprising: a first split oligonucleotide having an oligonucleotide sequence complementary to at least a portion of the second sequencing primer-binding site; and a second split oligonucleotide having an oligonucleotide sequence complementary to at least a portion of the second transposon end.

Sets of hybridization blockers, and kits including them, for use in massively parallel sequencing of a plurality of nucleic acid sequencing library molecules according to aspects of the present disclosure comprises a second pair of split hybridization blockers comprising: a first split oligonucleotide having an oligonucleotide sequence complementary to at least a portion of the complement of the second sequencing primer-binding site; and a second split oligonucleotide having an oligonucleotide sequence complementary to at least a portion of the complement of the second transposon end.

Sets of hybridization blockers, and kits including them, for use in massively parallel sequencing of a plurality of nucleic acid sequencing library molecules according to aspects of the present disclosure further comprises an oligonucleotide hybridization blocker comprising a nucleic acid sequence which is a complement of the first extension primer or an oligonucleotide hybridization blocker comprising the nucleic acid sequence of the first extension primer.

Sets of hybridization blockers, and kits including them, for use in massively parallel sequencing of a plurality of nucleic acid sequencing library molecules according to aspects of the present disclosure further comprises an oligonucleotide hybridization blocker comprising a nucleic acid sequence which is a complement of the second extension primer or an oligonucleotide hybridization blocker comprising the nucleic acid sequence of the second extension primer.

Sets of hybridization blockers, and kits including them, for use in massively parallel sequencing of a plurality of nucleic acid sequencing library molecules according to aspects of the present disclosure is provided wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers: 1) a first hybridization blocker comprising an oligonucleotide sequence which comprises at least a portion of a complement of the first extension primer-binding site; 2) a second hybridization blocker comprising at least a portion of the oligonucleotide sequence of the first sequencing primer-binding site; 3) a third hybridization blocker comprising at least a portion of the oligonucleotide sequence of the first transposon end; 4) a fourth hybridization blocker comprising an oligonucleotide sequence which is a complement of at least a portion of the second sequencing primer-binding site; 5) a fifth hybridization blocker comprising at least a portion of the oligonucleotide sequence of the second extension primer-binding site; 6) a sixth hybridization blocker comprising at least a portion of the oligonucleotide sequence of portion A of the third sequencing primer-binding site; 7) a seventh hybridization blocker comprising the oligonucleotide sequence of portion B of the third sequencing primer-binding site; 8) an eighth hybridization blocker comprising an oligonucleotide which is the complement of portion A of the fourth sequencing primer-binding site; and 9) a ninth hybridization blocker comprising an oligonucleotide which is the complement of portion B of the fourth sequencing primer-binding site. According to aspects of the present disclosure, each oligonucleotide of each of the nine hybridization blockers comprises a plurality of Tₘ increasing nucleotide analogs.

Sets of hybridization blockers, and kits including them, for use in massively parallel sequencing of a plurality of nucleic acid sequencing library molecules according to aspects of the present disclosure is provided wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers: 1) a first hybridization blocker comprising an oligonucleotide sequence which hybridizes to the complement of the first extension primer-binding site; 2) a second hybridization blocker comprising the oligonucleotide sequence which is the complement of the first sequencing primer-binding site; 3) a third hybridization blocker comprising the oligonucleotide sequence hybridizes to the first transposon end; 4) a fourth hybridization blocker comprising an oligonucleotide sequence which hybridizes to the complement of the second sequencing primer-binding site; 5) a fifth hybridization blocker comprising the oligonucleotide sequence which is the complement of the second extension primer-binding site; 6) a sixth hybridization blocker comprising an oligonucleotide sequence which hybridizes to portion A of the third sequencing primer-binding site; 7) a seventh hybridization blocker comprising an oligonucleotide sequence which hybridizes to portion B of the third sequencing primer-binding site; 8) an eighth hybridization blocker comprising an oligonucleotide which hybridizes to the complement of portion A of the fourth sequencing primer-binding site; and 9) a ninth hybridization blocker comprising an oligonucleotide which hybridizes to the complement of portion B of the fourth sequencing primer-binding site. According to aspects of the present disclosure, each oligonucleotide of each of the nine hybridization blockers comprises a plurality of Tₘ increasing nucleotide analogs.

Sets of hybridization blockers, and kits including them, for use in massively parallel sequencing of a plurality of nucleic acid sequence library inserts of at least four types of nucleic acid sequencing library molecule are provided. The at least four types of nucleic acid sequencing library molecule include: 1) a first type of nucleic acid sequencing library molecule comprising, a nucleic acid transposon insert, the transposon insert disposed between, and bonded to, a first adapter and a second adapter, the first adapter comprising at least a first extension primer-binding oligonucleotide, a first sequencing primer-binding oligonucleotide, and a first transposon end sequence, the second adapter comprising at least a second extension primer-binding oligonucleotide, a second sequencing primer-binding oligonucleotide, and a second transposon end sequence, wherein at least one of the first adapter and the second adapter comprises an index oligonucleotide; 2) a second type of nucleic acid sequencing library molecule comprising a complement nucleic acid sequence of the first type of nucleic acid sequencing library molecule wherein the complement nucleic acid sequence of the first type of nucleic acid sequencing library molecule comprises a complement nucleic acid transposon insert, the complement transposon insert disposed between, and bonded to, a complement first adapter and a complement second adapter, the complement first adapter comprising at least a complement first extension primer-binding oligonucleotide, a complement first sequencing primer-binding oligonucleotide, and a complement first transposon end sequence, the complement second adapter comprising at least a complement second extension primer-binding oligonucleotide, a complement second sequencing primer-binding oligonucleotide, and a complement second transposon end sequence, wherein at least one of the complement first adapter and the complement second adapter comprises a complement index oligonucleotide; 3) a third type of nucleic acid sequencing library molecule comprising a nucleic acid insert, each nucleic acid insert disposed between, and bonded to, a third adapter and a fourth adapter, the third adapter comprising at least a third extension primer-binding oligonucleotide, and a third sequencing primer-binding oligonucleotide, the fourth adapter comprising at least a fourth extension primer-binding oligonucleotide, and a fourth sequencing primer-binding oligonucleotide, with the proviso that neither the third adapter nor the fourth adapter comprises a transposon end sequence, wherein at least one of the third adapter and the fourth adapter comprises an index oligonucleotide; and 4) a fourth type of nucleic acid sequencing library molecule comprising a complement nucleic acid sequence of the third type of nucleic acid sequencing library molecule wherein the complement nucleic acid sequence of the third type of nucleic acid sequencing library molecule comprises a complement nucleic acid insert, the complement nucleic acid insert disposed between, and bonded to, a complement third adapter and a complement fourth adapter, the complement third adapter comprising at least a complement third extension primer-binding oligonucleotide, and a complement third sequencing primer-binding oligonucleotide, the complement fourth adapter comprising at least a complement fourth extension primer-binding oligonucleotide, and a complement fourth sequencing primer-binding oligonucleotide, with the proviso that neither the complement third adapter nor the complement fourth adapter comprises a transposon end sequence, wherein at least one of the complement third adapter and the complement fourth adapter comprises a complement index oligonucleotide. According to aspects of the present disclosure, such kits comprise at least nine blocking oligonucleotides, wherein each of the at least nine blocking oligonucleotides comprises at least one Tm increasing nucleotide, the at least nine blocking oligonucleotides comprising: 1) a first hybridization blocker comprising an oligonucleotide sequence which is the complement of the first extension primer-binding site; 2) a second hybridization blocker comprising the oligonucleotide sequence of the first sequencing primer-binding site; 3) a third hybridization blocker comprising the oligonucleotide sequence of the first transposon end; 4) a fourth hybridization blocker comprising an oligonucleotide sequence which is the complement of the second sequencing primer-binding site; 5) a fifth hybridization blocker comprising the oligonucleotide sequence of the second extension primer-binding site; 6) a sixth hybridization blocker comprising the oligonucleotide sequence of portion A of the third sequencing primer-binding site; 7) a seventh hybridization blocker comprising the oligonucleotide sequence of portion B of the third sequencing primer-binding site; 8) an eighth hybridization blocker comprising an oligonucleotide which is the complement of portion A of the fourth sequencing primer-binding site; and 9) a ninth hybridization blocker comprising an oligonucleotide which is the complement of portion B of the fourth sequencing primer-binding site.

Sets of hybridization blockers, and kits including them, for use in massively parallel sequencing of a plurality of nucleic acid sequence library inserts of at least four types of nucleic acid sequencing library molecule are provided. The at least four types of nucleic acid sequencing library molecule include: 1) a first type of nucleic acid sequencing library molecule comprising, a nucleic acid transposon insert, the transposon insert disposed between, and bonded to, a first adapter and a second adapter, the first adapter comprising at least a first extension primer-binding oligonucleotide, a first sequencing primer-binding oligonucleotide, and a first transposon end sequence, the second adapter comprising at least a second extension primer-binding oligonucleotide, a second sequencing primer-binding oligonucleotide, and a second transposon end sequence, wherein at least one of the first adapter and the second adapter comprises an index oligonucleotide; 2) a second type of nucleic acid sequencing library molecule comprising a complement nucleic acid sequence of the first type of nucleic acid sequencing library molecule wherein the complement nucleic acid sequence of the first type of nucleic acid sequencing library molecule comprises a complement nucleic acid transposon insert, the complement transposon insert disposed between, and bonded to, a complement first adapter and a complement second adapter, the complement first adapter comprising at least a complement first extension primer-binding oligonucleotide, a complement first sequencing primer-binding oligonucleotide, and a complement first transposon end sequence, the complement second adapter comprising at least a complement second extension primer-binding oligonucleotide, a complement second sequencing primer-binding oligonucleotide, and a complement second transposon end sequence, wherein at least one of the complement first adapter and the complement second adapter comprises a complement index oligonucleotide; 3) a third type of nucleic acid sequencing library molecule comprising a nucleic acid insert, each nucleic acid insert disposed between, and bonded to, a third adapter and a fourth adapter, the third adapter comprising at least a third extension primer-binding oligonucleotide, and a third sequencing primer-binding oligonucleotide, the fourth adapter comprising at least a fourth extension primer-binding oligonucleotide, and a fourth sequencing primer-binding oligonucleotide, with the proviso that neither the third adapter nor the fourth adapter comprises a transposon end sequence, wherein at least one of the third adapter and the fourth adapter comprises an index oligonucleotide; and 4) a fourth type of nucleic acid sequencing library molecule comprising a complement nucleic acid sequence of the third type of nucleic acid sequencing library molecule wherein the complement nucleic acid sequence of the third type of nucleic acid sequencing library molecule comprises a complement nucleic acid insert, the complement nucleic acid insert disposed between, and bonded to, a complement third adapter and a complement fourth adapter, the complement third adapter comprising at least a complement third extension primer-binding oligonucleotide, and a complement third sequencing primer-binding oligonucleotide, the complement fourth adapter comprising at least a complement fourth extension primer-binding oligonucleotide, and a complement fourth sequencing primer-binding oligonucleotide, with the proviso that neither the complement third adapter nor the complement fourth adapter comprises a transposon end sequence, wherein at least one of the complement third adapter and the complement fourth adapter comprises a complement index oligonucleotide. According to aspects of the present disclosure, such kits comprise at least nine blocking oligonucleotides, wherein each of the at least nine blocking oligonucleotides comprises at least one Tm increasing nucleotide, the at least nine blocking oligonucleotides comprising: 1) a first hybridization blocker comprising an oligonucleotide sequence which hybridizes to the complement of the first extension primer-binding site; 2) a second hybridization blocker comprising the oligonucleotide sequence which is the complement of the first sequencing primer-binding site; 3) a third hybridization blocker comprising the oligonucleotide sequence hybridizes to the first transposon end; 4) a fourth hybridization blocker comprising an oligonucleotide sequence which hybridizes to the complement of the second sequencing primer-binding site; 5) a fifth hybridization blocker comprising the oligonucleotide sequence which is the complement of the second extension primer-binding site; 6) a sixth hybridization blocker comprising an oligonucleotide sequence which hybridizes to portion A of the third sequencing primer-binding site; 7) a seventh hybridization blocker comprising an oligonucleotide sequence which hybridizes to portion B of the third sequencing primer-binding site; 8) an eighth hybridization blocker comprising an oligonucleotide which hybridizes to the complement of portion A of the fourth sequencing primer-binding site; and 9) a ninth hybridization blocker comprising an oligonucleotide which hybridizes to the complement of portion B of the fourth sequencing primer-binding site. According to aspects of the present disclosure, each oligonucleotide of each of the nine hybridization blockers comprises a plurality of Tₘ increasing nucleotide analogs.

Sets of hybridization blockers, and kits including them, for use in massively parallel sequencing of a plurality of nucleic acid sequencing library molecules are provided according to aspects of the present disclosure, the plurality of nucleic acid sequencing library molecules each having a template strand and a complementary strand, the template strand comprising an insert disposed between a first adapter and a second adapter, the insert having a first end and a second end, the first adapter comprising a first primer-binding site and a first target oligonucleotide, the first adapter optionally comprising a first index disposed between the first primer-binding site and the first target oligonucleotide, the first target oligonucleotide disposed between the first index, if present, and the first end of the insert, or the first target oligonucleotide disposed between the first primer-binding site and the first insert end, the second adapter comprising a second target oligonucleotide and a second primer-binding site, the second adapter optionally comprising a second index disposed between the second target oligonucleotide and the second primer-binding site, the second target oligonucleotide disposed between the second end of the insert and the second index, if present, or the second target oligonucleotide disposed between the second target nucleotide and the second primer-binding site, wherein the template strand comprises at least the first or the second index, the complementary strand being the complement of the template strand, the kit comprising: a set of hybridization blockers, each hybridization blocker comprising an oligonucleotide having a nucleic acid sequence which can hybridize to one of: the first target oligonucleotide, the second target oligonucleotide, the complement of the first target oligonucleotide, or the complement of the second target oligonucleotide, each oligonucleotide of each hybridization blocker comprising a plurality of Tₘ increasing nucleotide analogs, wherein the set of hybridization blockers comprises a first set of at least two hybridization blockers that comprise a first pair of split hybridization blockers, each pair of split hybridization blockers comprising a first split oligonucleotide having an oligonucleotide sequence complementary to one of: a portion A of either the first target oligonucleotide or the complement of portion A in the complementary strand, and a second split oligonucleotide having an oligonucleotide sequence complementary to one of: a portion B of the first target oligonucleotide or the complement of portion B in the complementary strand, wherein the first split oligonucleotide and the second split oligonucleotide each hybridize to either the portion A and the portion B on the template strand, or the complement of the portion A and the complement of the portion B on the complementary strand, and wherein the portion A and the portion B of the first target oligonucleotide are different, wherein the set of hybridization blockers further comprises a second pair of split hybridization blockers, the second pair comprising a first split oligonucleotide having an oligonucleotide sequence complementary to one of: a portion C of either the second target oligonucleotide or the complement of portion C in the complementary strand, and a second split oligonucleotide having an oligonucleotide sequence complementary to one of: a portion D of the second target oligonucleotide or the complement of portion D in the complementary strand, wherein the first split oligonucleotide and the second split oligonucleotide of the second pair each hybridize to either the portion C and the portion D on the template strand, or the complement of the portion C and the complement of the portion D on the complementary strand, and wherein the portion C and the portion D of the second target oligonucleotide are different, and, wherein, if the first pair of split hybridization blockers hybridizes to the portion A and the portion B of the template strand, then the second pair of split hybridization blockers hybridizes to the complement of the portion C and the complement of the portion D of the complementary strand, and, if the first pair of split hybridization blockers hybridizes to the complement of the portion A and the complement of the portion B of the complementary strand, then the second pair of split hybridization blockers hybridizes to the portion C and the portion D of the template strand. According to aspects of the present disclosure, the portion A and the portion B of the first target oligonucleotide are adjacent. According to aspects of the present disclosure, the portion C and the portion D of the second target oligonucleotide are adjacent. According to aspects of the present disclosure, each oligonucleotide of each of the nine hybridization blockers comprises a plurality of Tₘ increasing nucleotide analogs.

Sets of hybridization blockers, and kits including them, according to aspects of the present disclosure are provided wherein each of the hybridization blockers comprises at least one Tₘ increasing nucleotide, the set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, the set of hybridization blockers comprising: a first pair of split hybridization blockers, comprising a first split oligonucleotide having an oligonucleotide sequence complementary to a portion A of a first target oligonucleotide included in the template strand or the complementary strand and a second split oligonucleotide having an oligonucleotide sequence complementary to a portion B of the first target oligonucleotide included in the template strand or the complementary strand, wherein portion A and portion B are adjacent in the target oligonucleotide, such that the first pair of split hybridization blockers are adjacent and non-overlapping when hybridized to the first target oligonucleotide, wherein the first target oligonucleotide does not comprise an entire index sequence or a complement of an entire index sequence; and a second hybridization blocker comprising an oligonucleotide having an oligonucleotide sequence complementary to a second target oligonucleotide included in the template strand or the complementary strand, wherein the second target oligonucleotide does not comprise an entire Y-stem sequence, a complement of an entire Y-stem sequence, an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand. According to aspects of the present disclosure, the second hybridization blocker is a second pair of split hybridization blockers comprising a third split oligonucleotide having an oligonucleotide sequence complementary to a portion C of a second target oligonucleotide included in the template strand or the complementary strand and a fourth split oligonucleotide having an oligonucleotide sequence complementary to a portion D of the second target oligonucleotide included in the template strand or the complementary strand, wherein portion C and portion D are adjacent in the second target oligonucleotide, such that the second pair of split hybridization blockers are adjacent and non-overlapping when hybridized to the second target oligonucleotide, wherein the second target oligonucleotide does not comprise an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand. According to aspects of the present disclosure, the set of hybridization blockers further comprises at least one, at least two, at least three, at least four, at least five, at least six, or at least seven, additional hybridization blockers.

Sets of hybridization blockers, and kits including them, according to aspects of the present disclosure are provided wherein none of the hybridization blockers overlaps with more than 1 to 12 bases of an index.

Sets of hybridization blockers, and kits including them, according to aspects of the present disclosure are provided wherein each oligonucleotide of each hybridization blocker comprises a plurality of Tₘ increasing nucleotide analogs.

Sets of hybridization blockers, and kits including them, according to aspects of the present disclosure are provided wherein each hybridization blocker a length in the range of 5 to 60 bases in length, and each hybridization blocker comprises 20-90% of Tₘ-increasing nucleotide analogs.

### BRIEF DESCRIPTION OF THE DRAWINGS

FIG. 1 illustrates general aspects of nucleic acid sequencing library molecules including an insert sequence, a first adapter, and a second adapter, the first adapter adjacent to one end of the insert sequence and the second adapter adjacent to the second end of the insert sequence. The illustrated first adapter includes a first sequencing primer-binding site, a first barcode+UMI (index), and a first extension primer-binding site; the illustrated second adapter includes a second sequencing primer-binding site, a second barcode+UMI (index), and a second extension primer-binding site; the template strand is shown on top and the complement strand is shown on the bottom; the nucleic acid sequencing library molecules may be dual index, i.e., include two index oligonucleotides, as shown in the example in FIG. 1.

FIG. 2 is a diagram, not drawn to scale, which illustrates a pair of “split” hybridization blockers according to aspects of the present disclosure included in a set of hybridization blockers in context with nucleic acid sequencing library molecules.

FIG. 3 is a diagram, not drawn to scale, which illustrates a pair of “split” hybridization blockers according to aspects of the present disclosure included in a set of hybridization blockers in context with nucleic acid sequencing library molecules.

FIG. 4A is a graph showing effects of an oligonucleotide hybridization blocker set composition according to aspects of the present disclosure including nine oligonucleotide hybridization blockers on the percentage of on-target bases when the oligonucleotide hybridization blocker set composition is included in the hybridization mix with one single plex TruSeq (TS) library at 500 ng or up to eight TruSeq libraries (each at 250 ng, for an 8-plex total of 2 micrograms in a single hybridization), compared to without any oligonucleotide hybridization blockers; as shown, the nine oligonucleotide hybridization blocker set blocks as effectively for the single plex TruSeq library when compared with either of: (i) any of the individual libraries of the 8-plex (each at 250 ng); or, (ii) the combined libraries (e.g., average) of the 8-plex totaling 2 micrograms.

FIG. 4B is a graph showing effects of an oligonucleotide hybridization blocker set composition according to aspects of the present disclosure including nine oligonucleotide hybridization blockers on the percentage of on-target bases when the oligonucleotide hybridization blocker set composition is included in the hybridization mix with one single plex Nextera (Nx) library (500 ng) or up to eight Nextera libraries (each at 250 ng, for an 8-plex total of 2 micrograms in a single hybridization), compared to without any oligonucleotide hybridization blockers; as shown, the nine oligonucleotide hybridization blocker mix blocks as effectively for the single plex Nextera library when compared with either of: (i) any of the individual libraries of the 8-plex (each at 250 ng); or, (ii) the combined libraries (e.g., average) of the 8-plex totaling 2 micrograms.

FIG. 4C is a graph showing effects of an oligonucleotide hybridization blocker set composition according to aspects of the present disclosure including nine oligonucleotide hybridization blockers on the percentage of on-target bases when the oligonucleotide hybridization blocker set composition is included in the hybridization mix of three different types of libraries: (i) a single plex Nextera (Nx) library (500 ng); (ii) a single plex TruSeq (TS) library (500 ng); and, (iii) a duplexed TruSeq library with a Nextera library (each at 250 ng), in the same hybridization mix, compared to a single plex TruSeq (500 ng) and a single plex Nextera (500 ng) hybridization mix without any oligonucleotide hybridization blockers; as shown, the nine oligonucleotide hybridization blocker set blocks approximately equally for the duplexed TruSeq and Nextera libraries at 250 ng each when duplexed in same hybridization mix, and when compared to the individual single plex TruSeq and Nextera libraries at 500 ng each, block better than the Nextera library at 500 ng and only slightly less than the TruSeq library at 500 ng.

FIG. 5A is a graph showing effects of an oligonucleotide hybridization blocker composition including nine oligonucleotide hybridization blockers on the percentage of on-target bases when the oligonucleotide hybridization blocker set composition is included in the hybridization mix with one single plex TruSeq (TS) library (at 500 ng), or up to eight TruSeq (TS) libraries (each at 250 ng, or an 8-plex of 2 micrograms in a single hybridization) compared to without any oligonucleotide hybridization blockers, where the TruSeq libraries include TruSeq adapters with no separation or 6 bp separation (0/6 bp) separation between P5/R1 and P7/R2 sequence regions, respectively; the nine oligonucleotide hybridization blocker mix blocks TruSeq adapters independently of index/UMI length.

FIG. 5B is a graph showing effects of an oligonucleotide hybridization blocker set composition including nine oligonucleotide hybridization blockers on the percentage of on-target bases when the oligonucleotide hybridization blocker composition is included in the hybridization mix with one single plex TruSeq (TS) library (500 ng) or up to eight TruSeq libraries (each at 250 ng, or an 8-plex of 2 micrograms in a single hybridization) compared to without any blockers where the TruSeq libraries include TruSeq adapters with 8 bp separation or 19 bp separation (8/19 bp) separation between P5/R1 and P7/R2 sequence regions, respectively; thus, the nine oligonucleotide hybridization blocker set blocks TruSeq adapters independently of index/UMI length.

FIG. 6 is a graph showing effects of an oligonucleotide hybridization blocker set composition including nine oligonucleotide hybridization blockers on the percentage of on-target bases when the oligonucleotide hybridization blocker composition is included in the hybridization mix with 8-plex of NEXFLEX libraries (250 ng of each library in the 8-plex mix) in varying amounts in the range of 500-2500 ng; the results show that the oligonucleotide hybridization blocker set composition can be used in capture hybridization mixtures with up to 2500 ng library DNA without loss of performance.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; and Herdewijn, P. (Ed.), Oligonucleotide Synthesis: Methods and Applications, Methods in Molecular Biology, Humana Press, 2004.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

Compositions and methods for use in massively parallel sequencing of a plurality of nucleic acid sequencing library molecules are provided according to aspects of the present disclosure which include a set of hybridization blockers including oligonucleotides for inhibiting off-target hybridization and methods of their use.

The term “massively parallel sequencing,” including “sequencing by synthesis,” and also referred to as “next generation sequencing” and “high throughput sequencing,” indicates a process of simultaneous or near-simultaneous sequencing of many nucleic acid sequencing library molecules, such as thousands of nucleic acid sequencing library molecules. Sets of hybridization blockers provided according to aspects of the present disclosure are described herein with particular reference in some cases to Illumina Nextera and/or TruSeq platforms and configurations but are not limited thereto. Rather, the compositions and methods of the present disclosure may be used in any of various sequencing protocols and with various sequencing equipment, including, but not limited to, Ion Torrent systems.

According to aspects of the present disclosure, hybridization blockers included in a set of hybridization blockers include oligonucleotides. Oligonucleotides included in the hybridization blockers can be any length, depending on the length of the sequence to which the oligonucleotide will hybridize to function as a hybridization blocker.

According to particular aspects, a hybridization blocker includes an oligonucleotide having a length in the range of 5 to 60 bases in length, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60, bases in length. According to particular aspects, a hybridization blocker includes an oligonucleotide having a length in the range of 5 to 10 bases in length, such as 5, 6, 7, 8, 9, or 10 bases in length. According to particular aspects, a hybridization blocker includes an oligonucleotide having a length in the range of 11 to 15 bases in length, such as 11, 12, 13, 14, or 15 bases in length. According to particular aspects, a hybridization blocker includes an oligonucleotide having a length in the range of 16 to 20 bases in length, such as 16, 17, 18, 19, or 20 bases in length. According to particular aspects, a hybridization blocker includes an oligonucleotide having a length in the range of 21 to 25 bases in length, such as 21, 22, 23, 24, or 25 bases in length. According to particular aspects, a hybridization blocker includes an oligonucleotide having a length in the range of 26 to 30 bases in length, such as 26, 27, 28, 29, or 30 bases in length. According to particular aspects, a hybridization blocker includes an oligonucleotide having a length in the range of 31 to 35 bases in length, such as 31, 32, 33, 34, or 35 bases in length. According to particular aspects, a hybridization blocker includes an oligonucleotide having a length in the range of 36 to 40 bases in length, such as 36, 37, 38, 39, or 40 bases in length. According to particular aspects, a hybridization blocker includes an oligonucleotide having a length in the range of 41 to 45 bases in length, such as 41, 42, 43, 44, or 45 bases in length. According to particular aspects, a hybridization blocker includes an oligonucleotide having a length in the range of 46 to 50 bases in length, such as 46, 47, 48, 49, or 50 bases in length. According to particular aspects, a hybridization blocker includes an oligonucleotide having a length in the range of 51 to 55 bases in length, such as 51, 52, 53, 54, or 55 bases in length. According to particular aspects, a hybridization blocker includes an oligonucleotide having a length in the range of 56 to 60 bases in length, such as 56, 57, 58, 59, or 60 bases in length.

The oligonucleotides of the hybridization blockers each include at least one Tₘ increasing nucleotide analog.

According to aspects of the present disclosure, hybridization blockers included in a set of hybridization blockers hybridize to a “target oligonucleotide” sequence included in a plurality of nucleic acid sequencing library molecules. According to aspects of the present disclosure, the target oligonucleotide sequence to which a given hybridization blocker is directed, i.e., designed to hybridize to, is a portion of an adapter to which a library insert is attached. According to aspects of the present disclosure, the target oligonucleotide sequence to which a given hybridization blocker is directed, is designated by its function in a nucleic acid sequencing library molecule, e.g., a primer-binding sequence, an extension primer-binding sequence, a sequencing primer-binding sequence, or a transposon end. In addition to these elements, an adaptor may further include additional sequences, such as a sequence between any of a primer-binding sequence, an extension primer-binding sequence, and a sequencing primer-binding sequence between an insert and any of a primer-binding sequence, an extension primer-binding sequence, and a sequencing primer-binding sequence. According to aspects of the present disclosure, a Y-stem sequence is included between the insert and a sequencing primer-binding site. According to aspects of the present disclosure, a transposon end is included between the insert and a sequencing primer-binding site. These and other elements of nucleic acid sequencing library molecules are well-known in the art, and, according to aspects of the present disclosure, hybridization blockers included in a set of hybridization blockers may be directed to any element of a nucleic acid sequencing library molecule, or may span two or more elements, with the proviso that the hybridization blockers do not target the insert or index of a nucleic acid sequencing library molecule, or wherein none of the hybridization blockers overlaps with more than 1 to 12 bases of an index or insert.

According to aspects of the present disclosure, hybridization blockers included in a set of hybridization blockers may be directed to a target oligonucleotide sequence, which is longer, or shorter, than the full-length of the adapter element i.e., longer, or shorter than a primer-binding sequence, an extension primer-binding sequence, a sequencing primer-binding sequence, a transposon end, or Y-stem, with the proviso that the hybridization blockers do not target the insert or index of a nucleic acid sequencing library molecule, or wherein none of the hybridization blockers overlaps with more than 1 to 12 bases of an index or insert. Further, a pair of split hybridization blockers together may be directed to a target oligonucleotide sequence, which is longer, or shorter, than the full-length of the adapter element i.e., longer, or shorter than a primer-binding sequence, an extension primer-binding sequence, or a sequencing primer-binding sequence with the proviso that the pair of split hybridization blockers do not target the insert or index of a nucleic acid sequencing library molecule, or wherein none of the hybridization blockers overlaps with more than 1 to 12 bases of an index or insert

Hybridization blockers according to aspects of the present disclosure are not required to have the same length as the target oligonucleotide sequence. According to aspects of the present disclosure, when the hybridization blocker is hybridized to the target oligonucleotide sequence it sufficiently blocks undesired molecules from hybridizing to the target oligonucleotide sequence such that off-target hybridization capture is reduced.

According to aspects of the present disclosure, a hybridization blocker included in a set of hybridization blockers may be directed to a target oligonucleotide sequence, which is 1, 2, 3, 4, 5, 6, 7, 8, 9, 10, 11, 12, 13, 14, 15, 16, 17, 18, 19, 20, or more, bases longer than the full-length of the adapter element i.e., longer than a primer-binding sequence, an extension primer-binding sequence, a sequencing primer-binding sequence, a transposon end, or Y-stem, to which the hybridization blocker is directed, with the proviso that the hybridization blocker does not target the insert or index of a nucleic acid sequencing library molecule, or wherein none of the hybridization blockers overlaps with more than 1 to 12 bases of an index or insert. Where a hybridization blocker is longer than the target oligonucleotide sequence to which it is directed, i.e., longer than a primer-binding sequence, an extension primer-binding sequence, a sequencing primer-binding sequence, a transposon end, or Y-stem, the hybridization blocker may therefore extend to hybridize with at least a portion of a second element i.e., an adjacent primer-binding sequence, an extension primer-binding sequence, a sequencing primer-binding sequence, a transposon end, or Y-stem.

According to particular aspects, a pair of split hybridization blockers includes a first split hybridization blocker and a second split hybridization blocker wherein the pair of split hybridization blockers may be directed to a target oligonucleotide sequence, which is 1, 2, 3, 4, 5, 6, 7, 8, 9, 10, 11, 12, 13, 14, 15, 16, 17, 18, 19, 20, or more, bases longer than the full-length of the adapter element i.e., longer than a primer-binding sequence, an extension primer-binding sequence, a sequencing primer-binding sequence, a transposon end, or Y-stem, to which the pair of split hybridization blockers is directed, with the proviso that the pair of split hybridization blockers does not target the insert or index of a nucleic acid sequencing library molecule. According to particular aspects, either or both of a first split hybridization blocker and a second split hybridization blocker extends beyond the target oligonucleotide sequence, i.e., longer than a primer-binding sequence, an extension primer-binding sequence, a sequencing primer-binding sequence, a transposon end, or Y-stem, to which the pair of split hybridization blockers is directed and may therefore extend to hybridize with at least a portion of a second element i.e., an adjacent primer-binding sequence, an extension primer-binding sequence, a sequencing primer-binding sequence, a transposon end, or Y-stem, with the proviso that the pair of split hybridization blockers does not target the insert or index of a nucleic acid sequencing library molecule, or wherein none of the hybridization blockers overlaps with more than 1 to 12 bases of an index or insert

According to particular aspects, a pair of split hybridization blockers includes a first split hybridization blocker which hybridizes to the complement of a portion “A” of a sequencing primer-binding site and a second split hybridization blocker which hybridizes to the complement of a portion “B” of the sequencing primer-binding site wherein the second split hybridization blocker extends beyond the 5′ end of the complement of the sequencing primer-binding sequence into a Y-stem complement, such that 1, 2, 3, 4, 5, 6, 7, 8, 9, 10, 11, 12, 13, 14, 15, 16, 17, 18, 19, 20, or more, bases of the second split hybridization blocker hybridize with 1, 2, 3, 4, 5, 6, 7, 8, 9, 10, 11, 12, 13, 14, 15, 16, 17, 18, 19, 20, or more, bases of the 3′ end of the Y-stem complement.

According to particular aspects, a pair of split hybridization blockers includes a first split hybridization blocker which hybridizes to a portion “A” of a sequencing primer-binding site and a second split hybridization blocker which hybridizes to a portion “B” of the sequencing primer-binding site wherein the first split hybridization blocker extends beyond an end of the sequencing primer-binding sequence portion A into a Y-stem, such that 1, 2, 3, 4, 5, 6, 7, 8, 9, 10, 11, 12, 13, 14, 15, 16, 17, 18, 19, 20, or more, bases of the first split hybridization blocker hybridize with 1, 2, 3, 4, 5, 6, 7, 8, 9, 10, 11, 12, 13, 14, 15, 16, 17, 18, 19, 20, or more, bases of the Y-stem.

According to aspects of the present disclosure, a hybridization blocker included in a set of hybridization blockers may be directed to a target oligonucleotide sequence, which is 1, 2, 3, 4, 5, 6, 7, 8, 9, 10, 11, 12, 13, 14, 15, 16, 17, 18, 19, 20, or more, bases shorter than the full-length of the adapter element i.e., shorter than a primer-binding sequence, an extension primer-binding sequence, a sequencing primer-binding sequence, a transposon end, or Y-stem, to which the hybridization blocker is directed, with the proviso that the hybridization blocker does not target the insert or index of a nucleic acid sequencing library molecule, or wherein none of the hybridization blockers overlaps with more than 1 to 12 bases of an index or insert.

According to particular aspects, a pair of split hybridization blockers includes a first split hybridization blocker and a second split hybridization blocker wherein the pair of split hybridization blockers may be directed to a target oligonucleotide sequence, wherein the pair of split hybridization blockers is 1, 2, 3, 4, 5, 6, 7, 8, 9, 10, 11, 12, 13, 14, 15, 16, 17, 18, 19, 20, or more, bases shorter than the full-length of the adapter element i.e., shorter than a primer-binding sequence, an extension primer-binding sequence, a sequencing primer-binding sequence, a transposon end, or Y-stem, to which the pair of split hybridization blockers is directed, with the proviso that the pair of split hybridization blockers does not target the insert or index of a nucleic acid sequencing library molecule, or wherein none of the pair of split hybridization blockers overlaps with more than 1 to 12 bases of an index or insert. According to particular aspects, either or both of a first split hybridization blocker and a second split hybridization blocker do not extend beyond the target oligonucleotide sequence, e.g. both together are shorter than a primer-binding sequence, an extension primer-binding sequence, a sequencing primer-binding sequence, a transposon end, or Y-stem, to which the pair of split hybridization blockers is directed, with the proviso that the pair of split hybridization blockers does not target the insert or index of a nucleic acid sequencing library molecule.

According to particular aspects, a pair of split hybridization blockers includes a first split hybridization blocker which hybridizes to the complement of a portion “A” of a sequencing primer-binding site and a second split hybridization blocker which hybridizes to the complement of a portion “B” of the sequencing primer-binding site wherein neither the first split hybridization blocker nor the second split hybridization blocker extends beyond either the 3′ end or the 5′ end of the complement of the sequencing primer-binding sequence

According to particular aspects, a pair of split hybridization blockers includes a first split hybridization blocker which hybridizes to a portion “A” of a sequencing primer-binding site and a second split hybridization blocker which hybridizes to a portion “B” of the sequencing primer-binding site wherein neither the first split hybridization blocker nor the second split hybridization blocker extends beyond either the 3′ end or the 5′ end of the sequencing primer-binding sequence.

According to particular aspects, a pair of split hybridization blockers includes a first split hybridization blocker which hybridizes to the complement of a first sequencing primer-binding site and a second split hybridization blocker which hybridizes to the complement of a first transposon end wherein neither the first split hybridization blocker nor the second split hybridization blocker extends beyond either the 3′ end of the complement of the sequencing primer-binding sequence or the 5′ end of the complement of the transposon end.

According to further particular aspects, the first split hybridization blocker may hybridize both to the complement of the first sequencing primer-binding site and the complement of the first transposon end, while the second split hybridization blocker hybridizes to the complement of the first transposon end. According to further particular aspects, the first split hybridization blocker may hybridize to the complement of the first sequencing primer-binding site, while the second split hybridization blocker hybridizes both to the complement of the first sequencing primer-binding site and to the complement of the first transposon end.

According to particular aspects, a pair of split hybridization blockers includes a first split hybridization blocker which hybridizes to the complement of a second sequencing primer-binding site and a second split hybridization blocker which hybridizes to the complement of a second transposon end wherein neither the first split hybridization blocker nor the second split hybridization blocker extends beyond either the 5′ end of the complement of the sequencing primer-binding site or the 3′ end of the complement of the second transposon end. According to further particular aspects, the first split hybridization blocker may hybridize both to the complement of the second sequencing primer-binding site and the complement of the second transposon end, while the second split hybridization blocker hybridizes to the complement of the second transposon end. According to further particular aspects, the first split hybridization blocker may hybridize to the complement of the second sequencing primer-binding site, while the second split hybridization blocker hybridizes both to the complement of the second sequencing primer-binding site and to the complement of the second transposon end.

According to particular aspects, a pair of split hybridization blockers includes a first split hybridization blocker which hybridizes to a first sequencing primer-binding site and a second split hybridization blocker which hybridizes to a first transposon end wherein neither the first split hybridization blocker nor the second split hybridization blocker extends beyond either the 5′ end of the sequencing primer-binding sequence or the 3′ end of the first transposon end. According to further particular aspects, the first split hybridization blocker may hybridize both to the first sequencing primer-binding site and the first transposon end, while the second split hybridization blocker hybridizes to the first transposon end. According to further particular aspects, the first split hybridization blocker may hybridize to the first sequencing primer-binding site, while the second split hybridization blocker hybridizes both to the first sequencing primer-binding site and to the first transposon end.

According to particular aspects, a pair of split hybridization blockers includes a first split hybridization blocker which hybridizes to a second sequencing primer-binding site and a second split hybridization blocker which hybridizes to the second transposon end wherein neither the first split hybridization blocker nor the second split hybridization blocker extends beyond either the 3′ end of the sequencing primer-binding sequence or the 5′ end of the transposon end. According to further particular aspects, the first split hybridization blocker may hybridize both to the second sequencing primer-binding site and the second transposon end, while the second split hybridization blocker hybridizes to the second transposon end. According to further particular aspects, the first split hybridization blocker may hybridize to the second sequencing primer-binding site, while the second split hybridization blocker hybridizes both to the second sequencing primer-binding site and to the second transposon end.

According to particular aspects, a pair of split hybridization blockers includes a first split hybridization blocker and a second split blocker which have the same or similar length, such as where each of the first split hybridization blocker and a second split hybridization blocker have a length in the range of about 3 to about 30 bases, such as about 3 to 20 bases, such as about 3 to 10 bases, such as about 5 to 20 bases, such as about 4 to 25 bases, such as 3, 4, 5, 6, 7, 8, 9, 10, such as 11, 12, 13, 14, 15, such as 16, 17, 18, 19, 20, such as 21, 22, 23, 24, 25, such as 26, 27, 28, 29, or 30 bases.

According to particular aspects, a pair of split hybridization blockers includes a first split hybridization blocker and a second split hybridization blocker which have different lengths, such as where the first split hybridization blocker has a length in the range of about 3 to about 30 bases, such as about 3 to 20 bases, such as about 3 to 10 bases, such as about 5 to 20 bases, such as about 4 to 25 bases, such as 3, 4, 5, 6, 7, 8, 9, 10, such as 11, 12, 13, 14, 15, such as 16, 17, 18, 19, 20, such as 21, 22, 23, 24, 25, such as 26, 27, 28, 29, or 30, and the second split hybridization blocker has a length in the range of about 3 to about 30 bases, such as about 3 to 20 bases, such as about 3 to 10 bases, such as about 5 to 20 bases, such as about 4 to 25 bases, such as 3, 4, 5, 6, 7, 8, 9, 10, such as 11, 12, 13, 14, 15, such as 16, 17, 18, 19, 20, such as 21, 22, 23, 24, 25, such as 26, 27, 28, 29, or 30 bases.

The terms “Tₘ” and “melting temperature” are used interchangeably herein to refer to a temperature at which 50% (half) of population of double-stranded nucleic acid molecules become separated, i.e. single stranded. Methods for calculating Tₘ are well-known in the art.

The term “Tₘ increasing nucleotide analog” refers to a nucleotide analog that increases the melting temperature (Tₘ) of a double-stranded oligonucleotide that includes the nucleotide analog compared to the same double-stranded oligonucleotide without the nucleotide analog. A Tₘ increasing nucleotide analog may include a modified nucleobase, a modified sugar, a modified phosphate, or a combination of any two or more such modifications. Tₘ increasing nucleotide analogs include, but are not limited to, a locked nucleic acid (LNA) monomer, a peptide nucleic acid (PNA) monomer, and a bridged nucleic acid (BNA) monomer.

Any of various Tₘ increasing nucleotide analogs can be used including, but not limited to, an LNA monomer, PNA monomer, BNA monomer, or any two or more thereof, according to aspects of the present disclosure.

The term “LNA monomer” is used interchangeably herein with the term “LNA nucleotide” and refers to a nucleotide analog which includes a locked ribose due to presence of a covalent bond between the 2′ oxygen and the 4′ carbon of the ribose ring, that is, a 2′-O,4′-C-methylene-β-D-ribofuranosyl monomer which can be incorporated into an oligonucleotide, producing a “locked nucleic acid.” LNA monomers have similar, or improved, Watson-Crick base pairing selectivity compared to conventional nucleotides. Examples of LNA monomers include, but are not limited to, adenine bicyclonucleoside monomers, cytosine bicyclonucleoside monomers, guanine bicyclonucleoside monomers, 5-methylcytosine bicyclonucleoside monomers, thymine bicyclonucleoside monomers, and uracil bicyclonucleoside monomers. The Tm of an oligonucleotide including one or more LNA monomers is increased due to enhanced properties such as enhanced base stacking. Typically, the Tm of a is increased by two to eight ° C. for each LNA monomer incorporated into an LNA oligonucleotide. LNA monomers may be synthesized using well-known methods or obtained commercially. The terms “locked nucleic acid” and LNA are used interchangeably herein to refer to an oligonucleotide which includes one or more LNA monomers.

Any of various Tₘ increasing nucleotide analogs can be used including, but not limited to, 5-methyl dC, 2, 6-diaminopurine, propynyl-deoxyuridine, 5-hydroxybutynl-2′-deoxyuridine, and combinations of any two or more thereof according to aspects of the present disclosure. Any Tm increasing nucleotide analogs, and/or derivatives thereof, can be included so long as the derivatives retain the function of increasing the melting temperature (Tₘ) of a double-stranded oligonucleotide that includes the nucleotide analog compared to the same double-stranded oligonucleotide without the nucleotide analog.

According to aspects of the present disclosure, oligonucleotides of the hybridization blockers can have from about 20% to about 90% Tₘ increasing nucleotide analogs, such as from about 25% to about 85% Tₘ increasing nucleotide analogs, from about 30% to about 80% Tₘ increasing nucleotide analogs, from about 35% to about 80% Tₘ increasing nucleotide analogs, such as 35%, 40%, 50%, 55%, 60%, 65%, 70%, 75%, or 80% Tₘ increasing nucleotide analogs.

According to aspects of the present disclosure, oligonucleotides of the hybridization blockers can have from about 20% to about 90% LNA monomers, PNA monomers, BNA monomers, other Tₘ increasing nucleotide analogs, or any two or more thereof, such as from about 25% to about 85% LNA monomers, PNA monomers, BNA monomers, other Tₘ increasing nucleotide analogs, or any two or more thereof, from about 30% to about 80% LNA monomers, PNA monomers, BNA monomers, other Tₘ increasing nucleotide analogs, or any two or more thereof, from about 35% to about 80% LNA monomers, PNA monomers, BNA monomers, other Tₘ increasing nucleotide analogs, or any two or more thereof, such as 35%, 40%, 50%, 55%, 60%, 65%, 70%, 75%, or 80% LNA monomers, PNA monomers, BNA monomers, other Tₘ increasing nucleotide analogs, or any two or more thereof.

According to aspects of the present disclosure, oligonucleotides of the hybridization blockers can have from about 20% to about 90% locked nucleic acid (LNA) monomers, such as from about 25% to about 85% LNA monomers, from about 30% to about 80% LNA monomers, from about 35% to about 80% LNA monomers, such as 35%, 40%, 50%, 55%, 60%, 65%, 70%, 75%, or 80% LNA monomers.

In addition to Tₘ increasing nucleotide analogs, hybridization blockers of the present disclosure may include one or more nucleotide analogs in addition to Tₘ increasing nucleotide analogs. The term “nucleotide analog” in this context refers to a modified or non-naturally occurring nucleotide, particularly nucleotide analogs which can be polymerized, with naturally occurring nucleotides and/or non-naturally occurring nucleotides, by template-directed nucleic acid polymerization, or non-template-directed nucleic acid polymerization, catalyzed by a nucleic acid polymerase. Nucleotides and nucleotide analogs are well-known in the art. Particular nucleotide analogs are capable of Watson-Crick pairing via hydrogen bonds with a complementary nucleotide and illustratively include, but are not limited to, naturally-occurring and non-naturally-occurring nucleotides and analogs thereof including those containing an analog of a nucleotide base such as substituted purines or pyrimidines, deazapurines, methylpurines, methylpyrimidines, aminopurines, aminopyrimidines, thiopurines, thiopyrimidines, indoles, pyrroles, 7-deazaguanine, 7-deazaadenine, 7-methylguanine, hypoxanthine, pseudocytosine, pseudoisocytosine, isocytosine, isoguanine, 2-thiopyrimidines, 4-thiothymine, 6-thioguanine, nitropyrrole, nitroindole, and 4-methylindole. Nucleotide analogs include those containing an analog of a deoxyribose such as a substituted deoxyribose, a substituted or non-substituted arabinose, a substituted or non-substituted xylose, and a substituted or non-substituted pyranose. Nucleotide analogs include those containing an analog of a phosphate ester such as phosphorothioates, phosphorodithioates, phosphoroamidates, phosphoroselenoates, phosophoroanilothioates, phosphoroanilidates, phosphoroamidates, boronophosphates, phosphotriesters, and alkylphosphonates such as methylphosphonates.

According to aspects of the present disclosure, a hybridization blocker has a Tₘ in the range of 55° C. to 100° C., such as 60° C. to 100° C., such as 65° C. to 100° C., such as 70° C. to 100° C., such as 75° C. to 99° C., such as 75 to 90° C., such as 75° C. to 85° C., such as 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C.

According to aspects of the present disclosure, a set of hybridization blockers has a Tₘ in the range of 55° C. to 100° C., such as 60° C. to 100° C., such as 65° C. to 100° C., such as 70° C. to 100° C., such as 75° C. to 99° C., such as 75 to 90° C., such as 75° C. to 85° C., such as 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C.

According to aspects of the present disclosure, the 3′ end of one or more of the oligonucleotide hybridization blockers is optionally modified to prevent polymerase extension from the 3′ end, for example, in a post-hybridization capture PCR step. Any of various techniques for modifying the 3′ end of the oligonucleotide hybridization blockers is modified to prevent polymerase extension from the 3′ end can be used, including but not limited to, removal or modification of the hydroxyl group at the 3′ end of an oligonucleotide, such as to include a 3′ dideoxy-C(ddC), a phosphate group, or a 3′ spacer. According to aspects of the present disclosure, a spacer is present at the 3′ terminus of an oligonucleotide hybridization blocker. A nonlimiting example is a 3′ Spacer C3 (abbreviated 3SpC3), a 3 carbon chain (C3) which is attached to the terminal 3′ hydroxyl group of the oligonucleotide hybridization blocker. Alternatively, the 3′ end of the oligonucleotide hybridization blockers are not modified to prevent polymerase extension from the 3′ end, for example if no post-hybridization capture PCR step, or other polymerase-mediated extension step post-hybridization capture, is to be used.

The term “nucleotide” as used herein refers to a molecule including a nucleobase, sugar, and phosphate, as well as modified versions thereof, i.e. nucleotide analogs. Nucleotides are the monomeric units of a nucleic acid sequence, e.g. a DNA or RNA sequence. The term nucleotide includes ribonucleoside triphosphates, such as ATP, TTP, UTP, CTP, and GTP, and deoxyribonucleoside triphosphates, such as dATP, dCTP, dUTP, dGTP, and dTTP. Nucleotides are commonly referred to as A, T, G, C, or U as an abbreviation, in reference to the nucleobase. The term “nucleic acid” refers to RNA, DNA and RNA/DNA molecules having more than one nucleotide and/or nucleotide analog, in any form including single-stranded, double-stranded, oligonucleotide or polynucleotide.

Hybridization blockers according to aspects of the present disclosure may include one or more ribonucleoside triphosphates, one or more deoxyribonucleoside triphosphates, one or more Tm increasing nucleotide analogs, one or more nucleotide analogs, and mixtures of any two or more thereof. According to aspects of the present disclosure, a hybridization blocker may include RNA, DNA, RNA/DNA, or both RNA and DNA.

The terms “nucleotide sequence” and “nucleic acid sequence” are used to refer to the ordering of nucleotides in an oligonucleotide or polynucleotide.

The terms “hybridization” and “hybridized” refer to pairing and binding of complementary nucleic acids. Hybridization occurs to varying extents between two nucleic acids depending on factors such as the degree of complementarity of the nucleic acids, the melting temperature, Tₘ, of the nucleic acids and the stringency of hybridization conditions, and post-hybridization wash conditions, as is well-known in the art. The term “stringency of hybridization conditions” refers to conditions of temperature, ionic strength, and composition of a hybridization medium with respect to particular common additives such as surfactants, formamide, betaine, and polyethylene glycol. The term “stringency of post-hybridization wash conditions” refers to conditions of temperature, ionic strength, and composition of a hybridization medium, although additives such as surfactants, formamide, betaine, and polyethylene glycol are not commonly used in wash conditions. Particular surfactants that can be used include, for example, SDS (sodium dodecyl sulfate), sodium lauroyl sarcosinate (Sarkosyl), and CTAB (cetyltrimethylammonium bromide). Determination of particular hybridization conditions, and post-hybridization wash conditions, relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. High stringency post-hybridization wash conditions are those which only maintain hybridization of substantially complementary nucleic acids. The term “annealing” as used herein refers to the pairing and binding of an oligonucleotide to a target nucleic acid. There is no intended distinction between the terms “annealing” and “hybridizing.”

The term “complementary” as used herein encompasses, but is not limited to, Watson-Crick base pairing between nucleotides, between nucleotide analogs, or between nucleotides and nucleotide analogs, wherein nucleotides and/or nucleotide analogs are hydrogen bonded to one another, for example with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA-5′ is 100%, or completely, complementary to a region of the nucleotide sequence 5′-TTAGCTGG-3′. The term “complement” as used herein refers to a nucleotide sequence that is complementary to a given nucleotide sequence.

Typically, nucleic acids having about 85-100% complementarity are considered highly complementary, i.e. substantially complementary, and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize. The terms “specific hybridization” and “specifically hybridizes” refer to hybridization of a particular nucleic acid to a target nucleic acid without substantial hybridization to nucleic acids other than the target nucleic acid in a sample.

Hybridization

According to aspects of methods of the present disclosure, methods of massively parallel sequencing include annealing a set of oligonucleotide hybridization blockers to a plurality of nucleic acid sequencing library molecules.

Annealing a set of oligonucleotide hybridization blockers to a plurality of nucleic acid sequencing library molecules is performed at an annealing temperature. The annealing temperature depends on factors, including the nucleic acid sequence of the oligonucleotide hybridization blockers, and the target nucleic acid, and the composition of the reaction medium, including factors such as salt concentration, and concentration of additives, such as but not limited to, formamide, betaine, polyethylene glycol, a surfactant, such as SDS, and DMSO. Typically, the annealing temperature is in the range of 30° C. to 65° C., but can be higher or lower. The annealing temperature may be higher or lower than the oligonucleotide hybridization blocker melting temperature (Tₘ), however, typically hybridization blockers of a hybridization blocker set according to aspects of the present disclosure have a higher Tₘ than the annealing temperature to ensure that the hybridization blockers are bound, i.e. hybridized, to their targets during annealing. Similarly, one or more post-hybridization washes are performed at a temperature which is lower than the Tₘ of the hybridization blockers of a hybridization blocker set.

Hybridization blockers according to aspects of the present disclosure are present in particular ratios to target oligonucleotide sequences in nucleic acid sequencing library molecules during hybridization to reduce off-target capture according to aspects of the present disclosure, such as a molar ratio of hybridization blockers to target oligonucleotide sequence of 25:1, 20:1, 15:1, 10:1, 5:1, 4:1, 3:1, 2:1, 1:1, or a greater or lesser ratio.

According to particular aspects, the percentage of on-target performance is increased by at least about 5%, 10%, 15%, 20%, 25%, 30%, 35%, 40%, 50%, 60, 70%, 80%, 90%, 95%, or greater using hybridization blockers of the present disclosure, compared to no hybridization blockers.

Oligonucleotides

Oligonucleotides according to aspects of the present disclosure are generated synthetically using chemical synthetic and/or recombinant molecular biology techniques for example as detailed in Herdewijn, P. (Ed.), Oligonucleotide Synthesis: Methods and Applications, Methods in Molecular Biology, Humana Press, 2004.

Library Molecules

The term “starting nucleic acid” as used herein refers to nucleic acid, for example, DNA generally such as genomic DNA, mtDNA, DNA fragments, naturally occurring DNA, synthetic DNA, and RNA generally such as intact mRNA, fragmented mRNA, coding RNA, non-coding RNA, small RNA, miRNA, naturally occurring RNA, and synthetic RNA.

The starting nucleic acid, such as DNA or RNA, can be obtained from any source, including, but not limited to, a human, a non-human mammal, a vertebrate, an invertebrate, a microorganism, or a plant. The starting nucleic acid, such as DNA or RNA, can be obtained from one or more cells ex vivo or in vitro. For example, the starting nucleic acid, such as DNA or RNA, can be obtained from cultured cells, including, but not limited to, cell lines, primary cells or laboratory manipulated cells such as recombinant cells.

The starting nucleic acid, such as DNA or RNA, is typically present in a biological sample, which can be obtained from an individual, such as from a bodily sample, for example, blood, buccal swab, skin tissue, urine, saliva, tissue, and the like, and cell lines derived therefrom. A prenatal sample can be obtained from amniotic fluid, products of conception, blastocysts and blastomeres, corionic villi, fetal cells, fetal DNA, and/or fetal RNA circulating in maternal blood. Samples also be obtained from in vitro sources such as cell lines.

Biological samples can be obtained from any source, including, but not limited to, a human, a non-human mammal, a vertebrate, an invertebrate, a microorganism, or a plant. Biological samples can be obtained from one or more cells ex vivo or in vitro. For example, biological samples can be obtained from cultured cells, including, but not limited to, cell lines, primary cells, or laboratory manipulated cells such as recombinant cells.

Starting nucleic acid, such as DNA or RNA, is obtained by methods known in the art, for instance, as described in J. Sambrook and D. W. Russell, Molecular Cloning: A Laboratory Manual, Cold Spring Harbor Laboratory Press; 3rd Ed., 2001 or F. M. Ausubel, Ed., Short Protocols in Molecular Biology, Current Protocols; 5th Ed., 2002. Methods and oligonucleotides described herein may be used to generate libraries from nucleic acids of any of various organisms including, but not limited to, humans, non-human primates, rodents, rabbits, dogs, cats, horses, cattle, pigs, goats and sheep. Non-mammalian sources of sample nucleic acid can also be used, illustratively including fish and other aquatic organisms, birds, poultry, bacteria, viruses, plants, insects, reptiles, amphibians, fungi and mycobacteria. Thus, target nucleic acid, such as target DNA or target RNA, may be obtained from any of these sources.

Starting nucleic acid may also be obtained commercially and/or using commercial kits for isolation of starting nucleic acid.

The starting nucleic acid is processed to produce fragments, providing nucleic acid of appropriate size, depending on the particular sequencing platform, typically in the range of about 200 to 500 base pairs in length. The ends of the fragments can be treated to produce flush termini, 5′-overhang termini, or 3′ overhang termini, as desired.

Adapters are added to the termini of the nucleic acid fragments, such as by ligation, incorporation via PCR, or tagmentation, producing nucleic acid sequencing library molecules including an insert nucleic acid, a first adapter, and a second adapter. Adapters include one or more functional sequences, such as a sequencing primer-binding sequence (also called a sequencing primer-binding site), an extension primer-binding sequence (also called an extension primer-binding site), and an index sequence (also called a barcode sequence or unique molecular identifier, UMI). In sequencing libraries generated using a transposase/transposon method, nucleic acid sequencing library molecules include transposon ends attached to and flanking the insert nucleic acid, also termed a transposon insert herein.

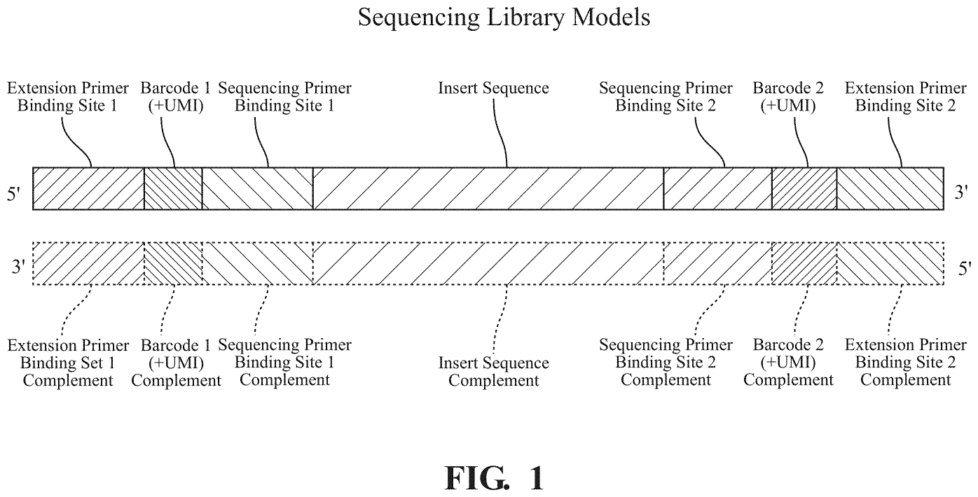

FIG. 1 illustrates general aspects of nucleic acid sequencing library molecules including an insert sequence, a first adapter, and a second adapter, the first adapter adjacent to one end of the insert sequence and the second adapter adjacent to the second end of the insert sequence. The illustrated first adapter includes a first sequencing primer-binding site, a first barcode+UMI (index), and a first extension primer-binding site. The illustrated second adapter includes a second sequencing primer-binding site, a second barcode+UMI (index), and a second extension primer-binding site. The template strand is shown on top and the complement strand is shown on the bottom. In some massively parallel sequencing techniques, nucleic acid sequencing library molecules are single index, i.e., include only one index, or the nucleic acid sequencing library molecules may be dual index, i.e., include two index oligonucleotides, as shown in the example in FIG. 1.

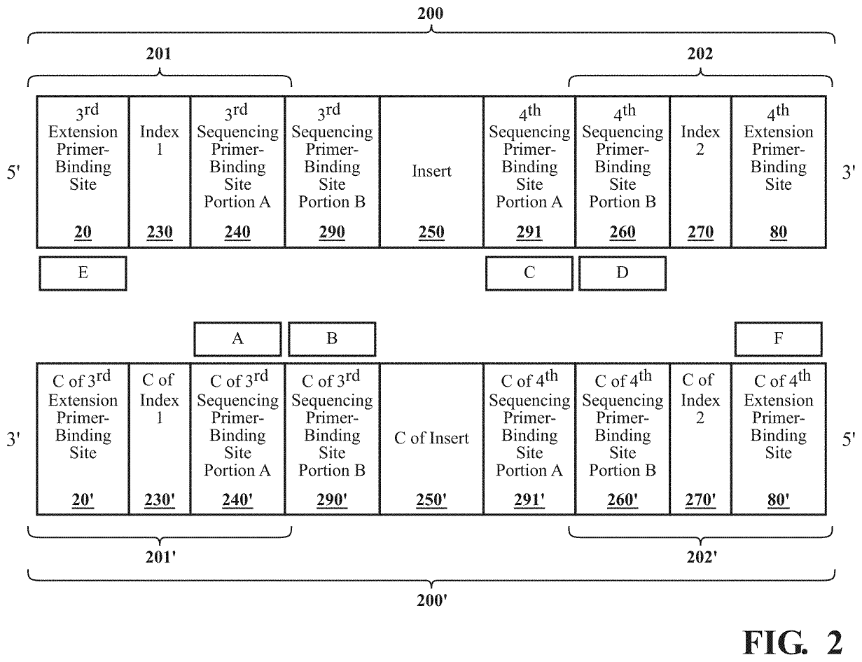

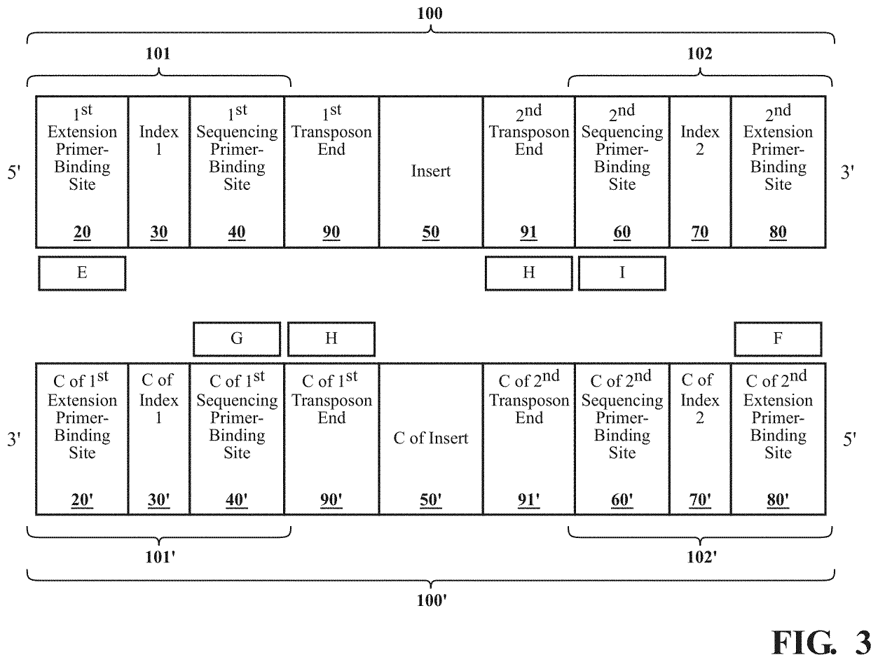

FIGS. 2 and 3 illustrate aspects of the present disclosure showing hybridization blockers included in a set of hybridization blockers in context, i.e. hybridized to target sequences of nucleic acid sequencing library molecules, thereby reducing hybridization of other sequences to the target sequences.

FIGS. 2 and 3, which are not drawn to scale, illustrate sets of hybridization blockers including two pairs of “split” hybridization blockers in each of FIGS. 2 and 3. FIG. 2 shows a first pair of split hybridization blockers labeled “A” and “B” and a second pair of split hybridization blockers labeled “C” and “D”. FIG. 3 shows a first pair of split hybridization blockers labeled “G” and “H” and a second pair of split hybridization blockers labeled “H” and “I”. The term “pair of split hybridization blockers” as used herein refers to two hybridization blockers including oligonucleotides which are complementary to, and therefore hybridize with, adjacent target oligonucleotides which are part of a nucleic acid sequencing library molecule.

The term “adjacent” as used herein refers to positioning of two oligonucleotides or polynucleotides with respect to each other, such as when describing positioning of two hybridization blockers including oligonucleotides when hybridized to a complementary target oligonucleotide or when describing two elements of a nucleic acid sequencing library molecule. The two oligonucleotides or polynucleotides are adjacent when separated by 0 to about 50 nucleotides, such as 0, 1, 2, 3, 4, 5, 6, 7, 8, 9, 10, 15, 20, 25, 30, 35, 40, 45, or 50 nucleotides. When separated by 0 nucleotides, the two oligonucleotides or polynucleotides directly abut one another.

According to preferred aspects of the present disclosure, the first split hybridization blocker and the second split hybridization blocker of a pair of split hybridization blockers are adjacent and separated by 0 nucleotides but not covalently bonded to each other when hybridized to their targets in the template strand or the complementary strand.

Surprisingly, it was found that hybridization of a pair of split hybridization blockers provides a “stacking interaction” between the two split hybridization blockers of the pair which are adjacent when hybridized to the template nucleic acid sequencing library molecule or its complement, such that the hybridization of both to their adjacent targets is more stable than if either of the two split hybridization blockers of the pair is hybridized to its target individually. This unexpected finding allows for advantageous manipulation of hybridization reaction conditions, such as reduction in the concentration of the hybridization blockers required to inhibit off-target hybridization events. Accordingly, aspects of the present disclosure include a set of hybridization blockers including at least two pairs of split hybridization blockers, wherein each pair of split hybridization blockers includes two oligonucleotide hybridization blockers which hybridize adjacent to each other on a complementary target oligonucleotide sequence of a template nucleic acid sequencing library molecule or its complement. The two pairs of split hybridization blockers may both hybridize to an oligonucleotide sequence of a template nucleic acid sequencing library molecule, both pairs may hybridize to the complement of the template nucleic acid sequencing library molecule, or one pair may hybridize to a target oligonucleotide sequence of a template nucleic acid sequencing library molecule and the other pair may hybridize to a target oligonucleotide in the complement of the template nucleic acid sequencing library molecule, with the proviso that both split hybridization blockers of a pair of split hybridization blockers hybridize to the same strand, either the template or its complement, adjacent to each other, and preferably with a 0 (zero) nucleotide separation between the hybridized split hybridization blockers.

Referring to FIG. 2, a template nucleic acid sequencing library molecule 200 is shown, along with its complement, 200′. Elements of the template nucleic acid sequencing library molecule 200 include an adapter 201 and an adapter 202. Adapter 201 comprises an extension primer-binding site 20, an index 230, and a sequencing primer-binding site comprising adjacent portions A 240 and B 290. Adaptors 201 and 202 are adjacent insert 250. Adapter 202 comprises a sequencing primer-binding site comprising adjacent portions A 291 and B 260, an index 270, and an extension primer-binding site 80. The complements of each of these elements are shown in 200′ where “C of” indicates “complement of.”

In further reference to FIG. 2, oligonucleotides “A” and “B” are a pair of split hybridization blockers which are complementary to two adjacent portions, 240′ and 290′, of a complement of a sequencing primer-binding site. Similarly, oligonucleotides “C” and “D” are a pair of split hybridization blockers which are complementary to two adjacent portions, 291 and 260, of a sequencing primer-binding site. FIG. 2 illustrates an exemplary aspect of the present disclosure, and the present disclosure further encompasses and contemplates variations to the aspect illustrated in FIG. 2, for example, where oligonucleotides “A” and “B” are a pair of split hybridization blockers which are complementary to two adjacent portions, 240 and 290 of a sequencing primer-binding site, and oligonucleotides “C” and “D” are a pair of split hybridization blockers which are complementary to two adjacent portions 290′ and 260′ of a complement of a sequencing primer-binding site.

Accordingly, it is appreciated that FIG. 2 illustrates aspects of the present disclosure in which one pair of the split hybridization blockers hybridizes to adjacent oligonucleotides in adapter 201′ and a second pair of the split hybridization blockers hybridizes to adjacent oligonucleotides in adapter 202 and that alternatively, and encompassed by the present disclosure, one pair of the split hybridization blockers hybridizes to adjacent oligonucleotides in adapter 201 and a second pair of the split hybridization blockers hybridizes to adjacent oligonucleotides in adapter 202′.

Referring to FIG. 3, a template nucleic acid sequencing library molecule 100 is shown, along with its complement, 100′. Elements of the template nucleic acid sequencing library molecule 100 include an adapter 101 and an adapter 102. Adapter 101 includes an extension primer-binding site 20, an index 30, and a sequencing primer-binding site 40. Adaptors 101 and 102 are adjacent transposon ends 90 and 92, respectively, of insert 50. Adapter 102 includes a sequencing primer-binding site 60, an index 70, and an extension primer-binding site 80. The complements of each of these elements is shown in 100′ where “C of” indicates “complement of”

In further reference to FIG. 3, oligonucleotides “G” and “H” are a pair of split hybridization blockers which are complementary to two adjacent target oligonucleotides, 40′ and 90′, of the complement 100′. Similarly, oligonucleotides “H” and “I” are a pair of split hybridization blockers which are complementary to two adjacent portions, 91 and 60, of the template nucleic acid sequencing library molecule 100. The present disclosure also thus contemplates oligonucleotides “G” and “H” are a pair of split hybridization blockers which are complementary to two adjacent portions, 40 and 90, of the template nucleic acid sequencing library molecule 100, and oligonucleotides “H” and “I” are a pair of split hybridization blockers which are complementary to two adjacent portions, 91′ and 60′ of the complement 100′.

Accordingly, it is appreciated that FIG. 3 illustrates aspects of the present disclosure in which one pair of the split hybridization blockers hybridizes to adjacent oligonucleotides in adapter 101′ and a second pair of the split hybridization blockers hybridizes to adjacent oligonucleotides in adapter 102 and that alternatively, and encompassed by the present disclosure, one pair of the split hybridization blockers hybridizes to adjacent oligonucleotides in adapter 101 and a second pair of the split hybridization blockers hybridizes to adjacent oligonucleotides in adapter 102′.

FIGS. 2 and 3 each further illustrate additional hybridization blockers included in a hybridization blocker set according to aspects of the present disclosure. In particular, FIG. 2 shows hybridization blocker “E” which is complementary to an extension primer-binding site 20, and hybridization blocker “F” which is complementary to a complement of an extension primer-binding site 80′. FIG. 3 also shows hybridization blocker “E” which is complementary to an extension primer-binding site 20, and hybridization blocker “F” which is complementary to a complement of an extension primer-binding site 80′.

It is appreciated that FIG. 2 illustrates aspects of the present disclosure in which hybridization blocker “E” hybridizes to an extension primer-binding site 20 in adapter 201 and a hybridization blocker “F” hybridizes to an extension primer-binding site in adapter 202′ and that alternatively, and encompassed by the present disclosure, the complement of hybridization blocker “E” is provided to hybridize to 20′ in adapter 201′ and the complement of hybridization blocker “F” is provided to hybridize to 80 in adapter 202.

It is appreciated that FIG. 3 illustrates aspects of the present disclosure in which hybridization blocker “E” hybridizes to an extension primer-binding site 20 in adapter 101 and a hybridization blocker “F” hybridizes to the complement of extension primer-binding site 80, i.e. 80′, in adapter 102′ and that alternatively, and encompassed by the present disclosure, the complement of hybridization blocker “E” is provided to hybridize to 20′ in adapter 101′ and the complement of hybridization blocker “F” is provided to hybridize to 80 in adapter 102.

According to aspects of the present disclosure, a hybridization blocker set is suitable for use in a multiplex hybridization including nucleic acid sequencing library molecules of different types, e.g., for example, nucleic acid sequencing library molecules generated using a transposon-based method, such as a Nextera™ technology, wherein the nucleic acid sequencing library molecule includes an insert disposed between two transposons mixed with nucleic acid sequencing library molecules without transposons.

According to aspects of the present disclosure, nucleic acid sequencing library molecules of different types included in a multiplex hybridization include common elements which are identical, or nearly identical, such that the same hybridization blockers will hybridize to the common elements of all of the nucleic acid sequencing library molecules of different types. In a non-limiting example, the nucleic acid sequencing library molecules of different types all include a P5 extension primer-binding site, or its complement, and a P7 extension primer-binding site, or its complement. As illustrated in FIGS. 2 and 3, this allows for inclusion of some hybridization blockers which hybridize to elements of nucleic acid sequencing library molecules of different types included in a multiplex hybridization. In FIGS. 2 and 3, the extension primer-binding sites 20 are identical, or nearly identical such that hybridization blocker “E” hybridizes to both. Similarly, in FIGS. 2 and 3, the extension primer-binding sites 80 are identical, or nearly identical such that hybridization blocker F hybridizes to the complement, i.e., 80′, of both.

Thus, it will be understood by reference to FIGS. 2 and 3, and as described herein, that a multiplex hybridization includes nucleic acid sequencing library molecules of different types such as a multiplex hybridization of the nucleic acid sequencing library molecules exemplified in FIGS. 2 and 3. Non-limiting examples of such nucleic acid sequencing library molecules which are combined for multiplex hybridization accord to aspects of the present disclosure are commonly described by commercial names including Nextera, and TruSeq, for example.

As exemplified in FIGS. 2 and 3 and with continued reference to both Figures, the present disclosure thus contemplates a plurality of nucleic acid sequencing library molecules, each having a template strand and a complementary strand. The template strand comprises an insert disposed between a first adapter and a second adapter, and the complementary strand comprises a complement of the insert disposed between a complement of the first adapter and a complement of the second adapter. FIGS. 2 and 3 each disclose a set of hybridization blockers where each hybridization blocker comprises an oligonucleotide having a nucleic acid sequence which is a complement of an oligonucleotide present in the template strand and/or complementary strand of at least some of the plurality of nucleic acid sequencing library molecules; provided, however, that none of the hybridization blockers comprises an oligonucleotide having a nucleic acid sequence which is a complement of the insert sequence, or an index sequence, if present, of the nucleic acid sequencing library molecules. According to aspects of the present disclosure, each oligonucleotide of each hybridization blocker comprises a plurality of Tₘ increasing nucleotide analogs.

With continued reference to FIGS. 2, and 3, it can be further understood that the present disclosure contemplates that the aforementioned hybridization blockers comprise at least two pairs of split hybridization blockers (e.g., FIG. 2, “A” & “B”, and “C” & “D”; and, FIG. 3, “G” & “H” and “H” & “I”), where each pair of split hybridization blockers comprise a first split oligonucleotide having an oligonucleotide sequence complementary to a portion A of a target oligonucleotide included in the template strand or the complementary strand and a second split oligonucleotide having an oligonucleotide sequence complementary to a portion B of the target oligonucleotide included in the template strand or the complementary strand, wherein portion A and portion B are adjacent in the target oligonucleotide, and wherein the portion A and the portion B of the target oligonucleotide to which each pair of split hybridization blockers hybridizes, is different.

As further shown in FIGS. 2 and 3, the first adapter comprises a first extension primer-binding site and a first sequencing primer-binding site, and the second adapter comprises a second extension primer-binding site and a second sequencing primer-binding site. Further, the complement of the first adapter comprises a complement of the first extension primer-binding site and a complement of the first sequencing primer-binding site, and the complement of the second adapter comprises a complement of the second extension primer-binding site and a complement of the second sequencing primer-binding site.

With reference to FIG. 2, a first pair of split hybridization blockers (e.g., blockers “A” & “B”, FIG. 2) comprises a first split oligonucleotide having an oligonucleotide sequence complementary to a portion A of the first sequencing primer-binding site and a second split oligonucleotide having an oligonucleotide sequence complementary to a portion B of the first sequencing primer-binding site, or wherein the first pair of split hybridization blockers comprises a first split oligonucleotide having an oligonucleotide sequence complementary to a portion A of the complement of the first sequencing primer-binding site and a second split oligonucleotide having an oligonucleotide sequence complementary to a portion B of the complement of the first sequencing primer-binding site.

Also shown in FIG. 2 is a second pair of split hybridization blockers (e.g., “C” & “D”) where the second pair comprises a first split oligonucleotide having an oligonucleotide sequence complementary to a portion A of the second sequencing primer-binding site and a second split oligonucleotide having an oligonucleotide sequence complementary to a portion B of the second sequencing primer-binding site, or wherein the second pair of split hybridization blockers comprises a first split oligonucleotide having an oligonucleotide sequence complementary to a portion A of the complement of the second sequencing primer-binding site and a second split oligonucleotide having an oligonucleotide sequence complementary to a portion B of the complement of the second sequencing primer-binding site.

Also exemplified in FIG. 2, is an aspect of the present disclosure wherein the hybridization blocker set also comprises an oligonucleotide hybridization blocker comprising a nucleic acid sequence which is a complement of the first extension primer (e.g., blocker “E”) or an oligonucleotide hybridization blocker comprising the nucleic acid sequence of the first extension primer; and, the hybridization blocker set further comprises an oligonucleotide hybridization blocker comprising a nucleic acid sequence which is a complement of the second extension primer (e.g., blocker “F”) or an oligonucleotide hybridization blocker comprising the nucleic acid sequence of the second extension primer.

In some embodiments, such as the embodiment of FIG. 2, the hybridization blocker set comprises oligonucleotides that block hybridization to the first adapter's extension primer-binding site, the complement of portion A and the complement of portion B of the first adapter, but do not block portion A or portion B of the sequencing primer-binding site of the first adapter or the complement of the first adapter's extension primer-binding site; and, block hybridization to the complement of the second adapter's extension primer-binding site, and portion A and portion B of the sequencing primer-binding site of the second adapter, but do not block the second adapter's extension primer-binding site, or the complement of portion A or portion B of the sequencing primer-binding site of the second adapter. Alternatively, the hybridization blocker set comprises oligonucleotides that block hybridization to the complement of the first adapter's extension primer-binding site, and portion A and portion B of the sequencing primer-binding site of the first adapter, but do not block hybridization to the complement of portion A or portion B of the sequencing primer-binding site of the first adapter or the first adapter's extension primer-binding site; and, block the second adapter's extension primer-binding site, and the complement of portion A and portion B of the sequencing primer-binding site of the second adapter, but do not block hybridization to the complement of the second adapter's extension primer-binding site, or portion A or portion B of the sequencing primer-binding site of the second adapter.

According to particular aspects of the present disclosure, a hybridization blocker set comprises: combination a) a first hybridization blocker which is the complement of, and therefore hybridizes to, a first target oligonucleotide sequence included in the first adapter of the template strand of a plurality of nucleic acid sequencing library molecules; and a first pair of split hybridization blockers which together are the complement of, and therefore hybridize to, a second target oligonucleotide sequence included in the first adapter of the complementary strand of the plurality of nucleic acid sequencing library molecules, wherein the first pair of split hybridization blockers are adjacent when hybridized to the second target oligonucleotide sequence, and wherein the first target oligonucleotide sequence is not a complement of, or identical to, the second target oligonucleotide sequence.

According to particular aspects of the present disclosure, a hybridization blocker set comprises: combination b) a first hybridization blocker which is the complement of, and therefore hybridizes to, a first target oligonucleotide sequence included in the first adapter of the complementary strand of a plurality of nucleic acid sequencing library molecules; and a first pair of split hybridization blockers which together are the complement of, and therefore hybridize to, a second target oligonucleotide sequence included in the first adapter of the template strand of the plurality of nucleic acid sequencing library molecules, wherein the first pair of split hybridization blockers are adjacent when hybridized to the second target oligonucleotide sequence, and wherein the first target oligonucleotide sequence is not a complement of, or identical to, the second target oligonucleotide sequence.

According to particular aspects of the present disclosure, a hybridization blocker set comprises: combination c) a first hybridization blocker which is the complement of, and therefore hybridizes to, a first target oligonucleotide sequence included in the second adapter of the template strand of a plurality of nucleic acid sequencing library molecules; and a first pair of split hybridization blockers which together are the complement of, and therefore hybridize to, a second target oligonucleotide sequence included in the second adapter of the complementary strand of the plurality of nucleic acid sequencing library molecules, wherein the first pair of split hybridization blockers are adjacent when hybridized to the second target oligonucleotide sequence, and wherein the first target oligonucleotide sequence is not a complement of, or identical to, the second target oligonucleotide sequence.

According to particular aspects of the present disclosure, a hybridization blocker set comprises: combination d) a first hybridization blocker which is the complement of, and therefore hybridizes to, a first target oligonucleotide sequence included in the second adapter of the complementary strand of a plurality of nucleic acid sequencing library molecules; and a first pair of split hybridization blockers which together are the complement of, and therefore hybridize to, a second target oligonucleotide sequence included in the second adapter of the template strand of the plurality of nucleic acid sequencing library molecules, wherein the first pair of split hybridization blockers are adjacent when hybridized to the second target oligonucleotide sequence, and wherein the first target oligonucleotide sequence is not a complement of, or identical to, the second target oligonucleotide sequence.

According to particular aspects of the present disclosure, a hybridization blocker set comprises, combination a) and combination c), or combination b) and combination d), or combination b) and combination c), or combination a) and combination d).

In embodiments such as shown in FIG. 3, the insert of the template strand comprises a transposon insert disposed between a first transposon end and a second transposon end, the first transposon end adjacent the first sequencing primer-binding site and the second transposon end adjacent the second sequencing primer-binding site, the complementary strand comprising a complement of the transposon insert, a complement of the first transposon end and a complement of the second transposon end. In such an embodiment, the first adapter comprises a first extension primer-binding site and a first sequencing primer-binding site, the second adapter comprises a second extension primer-binding site and a second sequencing primer-binding site, and the complement of the first adapter comprises a complement of the first extension primer-binding site and a complement of the first sequencing primer-binding site, and the complement of the second adapter comprises a complement of the second extension primer-binding site and a complement of the second sequencing primer-binding site. As also shown in FIG. 3, a first pair of split hybridization blockers (e.g., “G” & “H”) comprises a first split oligonucleotide having an oligonucleotide sequence complementary to at least a portion of the complement of the first sequencing primer-binding site and a second split oligonucleotide having an oligonucleotide sequence complementary to at least a portion of the complement of the first transposon end. Alternatively, envisioned are embodiments that include a first pair of split hybridization blockers that comprises a first split oligonucleotide having an oligonucleotide sequence complementary to at least a portion of the first sequencing primer-binding site and a second split oligonucleotide having an oligonucleotide sequence complementary to at least a portion of the first transposon end. Also exemplified in FIG. 3 is a second pair of split hybridization blockers (e.g., “H” & “I”) that comprises a first split oligonucleotide having an oligonucleotide sequence complementary to at least a portion of the second sequencing primer-binding site and a second split oligonucleotide having an oligonucleotide sequence complementary to at least a portion of the second transposon end. Accordingly, also disclosed is, alternatively, a second pair of split hybridization blockers that comprises a first split oligonucleotide having an oligonucleotide sequence complementary to at least a portion of the complement of the second sequencing primer-binding site and a second split oligonucleotide having an oligonucleotide sequence complementary to at least a portion of the complement of the second transposon end. In the FIG. 3 embodiment, the oligonucleotide that is the complement of the first transposon end is the same as the oligonucleotide that is the second transposon end, and thus the same hybridization blocker may be used for both; and, the oligonucleotide that is the first transposon end is the same as the oligonucleotide that is the complement of the second transposon end, and thus the same hybridization blocker may be used for both.

As shown in FIG. 3, the hybridization blocker set may also comprise an oligonucleotide hybridization blocker comprising a nucleic acid sequence which is a complement of the first extension primer-binding site, exemplified as hybridization blocker “E”, or an oligonucleotide hybridization blocker comprising the nucleic acid sequence of the first extension primer-binding site.

As shown in FIG. 3, the hybridization blocker set may also comprise an oligonucleotide hybridization blocker comprising a nucleic acid sequence which is a complement of the second extension primer, exemplified as hybridization blocker “F”, or an oligonucleotide hybridization blocker comprising the nucleic acid sequence of the second extension primer.

According to aspects of the present disclosure, the disclosed hybridization blocker set may comprise oligonucleotides for blocking hybridization to a first extension primer-binding site, a complement of the first sequencing primer-binding site, and a complement of the first transposon end, but not the complement of the first extension primer-binding site, the first sequencing primer-binding site, and the first transposon end; and, for blocking hybridization to the second transposon end, the second sequencing primer-binding site, and the complement of the second extension primer-binding site, but not the complement of the second transposon end, the complement of the second sequencing primer-binding site, or the second extension primer-binding site. It will thus be understood that disclosed is a hybridization blocker set that may comprise oligonucleotides for blocking hybridization to a complement of the first extension primer-binding site, a first sequencing primer-binding site, and the first transposon end, but not the first extension primer-binding site, the complement of the first sequencing primer-binding site, or the complement of the first transposon end; and, for blocking the complement of the second transposon end, the complement of the second sequencing primer-binding site, and the second extension primer-binding site, but not the second transposon end, the second sequencing primer-binding site, or the complement of the second extension primer-binding site.

According to aspects of the present disclosure, a disclosed hybridization blocker set includes oligonucleotides which block hybridization to only one of (i) or (ii): (i) a first extension primer-binding site, a complement of the first sequencing primer-binding site, and a complement of the first transposon end, or (ii) the complement of the first extension primer-binding site, the first sequencing primer-binding site, and the first transposon end; and, block hybridization only to a corresponding one of (iii) or (iv): (iii) the complement of the second transposon end, the complement of the second sequencing primer-binding site, or the second extension primer-binding site, or (iv) the second transposon end, the second sequencing primer-binding site, and the complement of the second extension primer-binding site. It will thus be understood that disclosed is a hybridization blocker set including oligonucleotides which block only one of (i) or (ii): (i) a complement of the first extension primer-binding site, a first sequencing primer-binding site, and the first transposon end, or (ii) the first extension primer-binding site, the complement of the first sequencing primer-binding site, or the complement of the first transposon end, and block only the corresponding one of (iii) or (iv): (iii) the second transposon end, the second sequencing primer-binding site, or the complement of the second extension primer-binding site, or (iv) the complement of the second transposon end, the complement of the second sequencing primer-binding site, and the second extension primer-binding site.

According to aspects of the present disclosure, hybridization blockers are illustrated in Table I in which +A, +C, +G, and +T designate LNA monomers.

TABLE I

| | | | Tm (° C.) | Tm (° C.) |
|---|---|---|---|---|
| | | Default | 750 mM | 15 mM |
| Name | Sequence | Tm (° C.) | Na⁺ | Na⁺ |
| SEQ ID | CA+AG+C+A+G+A+A+GA+C+G+G+CA+T+A+C+G+A+G+AT | 87.4 | 100 | 74.5 |
| NO: 1 | | | | |
| SEQ ID | A+T+CT+C+G+T+ATG+C+C+G+T+CT+T+C+T+G+CT+T+G | 88.0 | 100 | 75.0 |
| NO: 2 | | | | |
| SEQ ID | GT+GA+CT+G+GAG+T+T+CA+GA+CG+TG+TG+CT+CT+T+C+C+GA+TC | 93.6 | 100 | 80.5 |
| NO: 3 | | | | |
| SEQ ID | GA+T+C+G+GA+A+GAG+CA+CA+CGT+CT+GAA+CT+C+CA+GT+CA+C | 92.2 | 100 | 79.2 |
| NO: 4 | | | | |
| SEQ ID | AA+T+GA+TA+CGG+C+GA+C+CA+C+CGA+GAT+CTA+C+AC | 90.2 | 100 | 77.2 |
| NO: 5 | | | | |
| SEQ ID | G+T+GT+A+GA+T+CT+CG+GT+GG+T+CG+C+CG+T+AT+CA+TT | 90.4 | 100 | 77.3 |
| NO: 6 | | | | |
| SEQ ID | A+CA+CT+CTT+T+CC+CTA+CA+C+GA+CG+CT+CTT+CCG+ATC | 93.6 | 100 | 80.5 |
| NO: 7 | | | | |
| SEQ ID | GA+T+CG+GA+A+GAG+CG+T+CG+TG+T+A+G+G+GAA+A+G+AG+T+GT | 93.0 | 100 | 79.9 |
| NO: 8 | | | | |
| SEQ ID | C+T+GT+CT+CT+T+ATA+CA+CAT+CT+GA+CG+CTG+CC+GA+C+GA | 92.3 | 100 | 79.1 |
| NO: 9 | | | | |
| SEQ ID | C+T+GT+CT+CT+T+ATA+CA+C+AT+CT+CCG+AG+CC+CA+CG+A+GA+C | 93.5 | 100 | 80.3 |
| NO: 10 | | | | |
| SEQ ID | GT+GA+CT+G+GAG+T+T+CA+GA+C | 72.5 | 88.6 | 60.7 |
| NO: 11 | | | | |
| SEQ ID | G+TG+TG+CT+CT+T+C+C+GA+TC | 76.3 | 92.3 | 64.5 |
| NO: 12 | | | | |
| SEQ ID | A+CA+CT+CTT+T+CC+CTA+CA | 72.6 | 90.1 | 60.3 |
| NO: 13 | | | | |
| SEQ ID | +C+GA+CG+CT+CTT+CCG+ATC | 71.2 | 85.8 | 60.1 |
| NO: 14 | | | | |
| SEQ ID | G+T+GA+C+T+G+G+AG+T+T+C+A+GA+C | 82.9 | 100 | 70.5 |
| NO: 15 | | | | |
| SEQ ID | G+T+G+T+G+C+T+CT+T+C+C+GA+TC | 81.3 | 97.7 | 69.7 |
| NO: 16 | | | | |
| SEQ ID | A+CA+C+T+CT+T+T+C+C+CTA+CA | 78.3 | 96.4 | 65.6 |
| NO: 17 | | | | |
| SEQ ID | +C+GA+C+G+C+T+CT+T+C+C+G+ATC | 80.5 | 95.9 | 68.8 |
| NO: 18 | | | | |
| SEQ ID | G+T+GA+C+T+G+G+AG+T+T+C+A+GA+C+GT | 87.6 | 100 | 74.9 |
| NO: 19 | | | | |
| SEQ ID | +GA+T+C+G+GA+A+G+AG+CA+CA+C+GT | 84.6 | 100 | 72.2 |
| NO: 20 | | | | |
| SEQ ID | A+CA+CT+CT+T+T+C+C+C+TA+CA+C+GA | 85.9 | 100 | 72.9 |
| NO: 21 | | | | |
| SEQ ID | +GA+T+C+G+GA+A+G+AG+CG+T+C+GT | 83.5 | 99.7 | 71.3 |
| NO: 22 | | | | |
| SEQ ID | A+T+G+T+G+TA+TA+A+G+A+GA+C+AG | 74.0 | 93.0 | 61.2 |
| NO: 23 | | | | |
| SEQ ID | T+CG+T+CGG+C+AG+CG+T+C+AG | 82.8 | 97.3 | 71.3 |
| NO: 24 | | | | |
| SEQ ID | G+T+CT+CG+T+G+GG+CT+CG+GAG | 85.0 | 99.4 | 73.5 |
| NO: 25 | | | | |
| SEQ ID | C+T+G+T+C+T+CT+T+A+T+A+CA+C+AT | 72.3 | 91.2 | 59.6 |
| NO: 26 | | | | |
| SEQ ID | C+T+GA+CG+CTG+CC+GA+C+GA | 84.0 | 98.7 | 72.4 |
| NO: 27 | | | | |
| SEQ ID | CT+CCG+AG+CC+CA+CG+A+GA+C | 81.6 | 95.8 | 70.3 |
| NO: 28 | | | | |

With reference to hybridization blockers A, B, C, D, E, F, G, H, and I in FIGS. 2 and 3, the sequences of Table I include: SEQ ID NO:1 (complement of F); SEQ ID NO:2 (F); SEQ ID NO:3 (D & C); SEQ ID NO:4 (complement of D & C); SEQ ID NO:5 (complement of E); SEQ ID NO:6 (E); SEQ ID NO:7 (A & B); SEQ ID NO:8 (complement of A & B); SEQ ID NO:9 (complement of H & G); SEQ ID NO:10 (complement of H & I); SEQ ID NO:11 (D); SEQ ID NO:12 (C); SEQ ID NO:13 (A); SEQ ID NO:14 (B); SEQ ID NO:15 (alternative to SEQ ID NO:11 which can be interchanged with SEQ ID NO:11 in a set of hybridization blockers); SEQ ID NO:16 (alternative to SEQ ID NO:12 which can be interchanged with SEQ ID NO:12 in a set of hybridization blockers); SEQ ID NO:17 (alternative to SEQ ID NO:13 which can be interchanged with SEQ ID NO:13 in a set of hybridization blockers); SEQ ID NO:18 (alternative to SEQ ID NO:14 which can be interchanged with SEQ ID NO:14 in a set of hybridization blockers); SEQ ID NO:19 (alternative to SEQ ID NO:11 which can be interchanged with SEQ ID NO:11 in a set of hybridization blockers); SEQ ID NO:20 (complement NO:12); SEQ ID NO:21 (alternative to SEQ ID NO:13 which can be interchanged with SEQ ID NO:13 in a set of hybridization blockers); SEQ ID NO:22 (complement NO:14); SEQ ID NO:23 (H); SEQ ID NO:24 (G); SEQ ID NO:25 (I); SEQ ID NO:26 (complement of H); SEQ ID NO:27 (complement of G); and SEQ ID NO:28 (complement of I).

According to aspects of the present disclosure, a hybridization blocker set including at least nine hybridization blockers is provided for use in a multiplex hybridization including nucleic acid sequencing library molecules of different types.

According to aspects of the present disclosure, a hybridization blocker set including at least nine hybridization blockers is provided including, with reference to FIGS. 2 and 3:

a first hybridization blocker comprising an oligonucleotide sequence which is the complement of the first extension primer-binding site, see hybridization blocker “E” in FIGS. 2 and 3;
a second hybridization blocker comprising the oligonucleotide sequence of the first sequencing primer-binding site, see hybridization blocker “G” in FIG. 3;
a third hybridization blocker comprising the oligonucleotide sequence of the first transposon end, see hybridization blocker “H” in FIG. 3 and note that hybridization blocker “H” also hybridizes to the first transposon end;
a fourth hybridization blocker comprising an oligonucleotide sequence which is the complement of the second sequencing primer-binding site, see hybridization blocker “I” in FIG. 3;
a fifth hybridization blocker comprising the oligonucleotide sequence of the second extension primer-binding site, see hybridization blocker “F” in FIGS. 2 and 3;
a sixth hybridization blocker comprising the oligonucleotide sequence of portion A of the third sequencing primer-binding site, see hybridization blocker “A” in FIG. 2;
a seventh hybridization blocker comprising the oligonucleotide sequence of portion B of the third sequencing primer-binding site, see hybridization blocker “B” in FIG. 2;
an eighth hybridization blocker comprising an oligonucleotide which is the complement of portion A of the fourth sequencing primer-binding site, see hybridization blocker “C” in FIG. 2; and
a ninth hybridization blocker comprising an oligonucleotide which is the complement of portion B of the fourth sequencing primer-binding site, see hybridization blocker “D” in FIG. 2.

According to aspects of the present disclosure, a hybridization blocker set including at least nine hybridization blockers is provided including, with reference to FIGS. 2 and 3:

a first hybridization blocker comprising an oligonucleotide sequence which hybridizes to the complement of the first extension primer-binding site, i.e. the complement of the hybridization blocker “E” in FIGS. 2 and 3;
a second hybridization blocker comprising the oligonucleotide sequence which is the complement of the first sequencing primer-binding site, i.e. the complement of the hybridization blocker “G” in FIG. 3;
a third hybridization blocker comprising the oligonucleotide sequence hybridizes to the first transposon end, i.e. the complement of the hybridization blocker “H” in FIG. 3;
a fourth hybridization blocker comprising an oligonucleotide sequence which hybridizes to the complement of the second sequencing primer-binding site, i.e. the complement of the hybridization blocker “I” in FIG. 3;
a fifth hybridization blocker comprising the oligonucleotide sequence which is the complement of the second extension primer-binding site, i.e. the complement of the hybridization blocker “F” in FIGS. 2 and 3;
a sixth hybridization blocker comprising an oligonucleotide sequence which hybridizes to portion A of the third sequencing primer-binding site, i.e. the complement of the hybridization blocker “A” in FIG. 2;
a seventh hybridization blocker comprising an oligonucleotide sequence which hybridizes to portion B of the third sequencing primer-binding site, i.e. the complement of the hybridization blocker “B” in FIG. 2;
an eighth hybridization blocker comprising an oligonucleotide which hybridizes to the complement of portion A of the fourth sequencing primer-binding site, i.e. the complement of the hybridization blocker “C” in FIG. 2; and
a ninth hybridization blocker comprising an oligonucleotide which hybridizes to the complement of portion B of the fourth sequencing primer-binding site, i.e. the complement of the hybridization blocker “D” in FIG. 2.

According to aspects of the present disclosure, a hybridization blocker set including at least nine hybridization blockers, designated A, B, C, D, E, F, G, H, and I as illustrated in FIGS. 2 and 3, and alternatives thereof as described herein, is provided for use in a multiplex hybridization including nucleic acid sequencing library molecules of different types.

According to aspects of the present disclosure, a hybridization blocker set including at least nine hybridization blockers as illustrated in Table II in which +A, +C, +G, and +T designate LNA monomers.

TABLE II

| | |
|---|---|
| SEQ ID | A+T+CT+C+G+T+ATG+C+C+G+T+CT+T+C+T+G+CT+T+G |
| NO: 2 | |
| SEQ ID | G+T+GT+A+GA+T+CT+CG+GT+GG+T+CG+C+CG+T+AT+ |
| NO: 6 | CA+TT |
| SEQ ID | GT+GA+CT+G+GAG+T+T+CA+GA+C |
| NO: 11 | |
| SEQ ID | G+TG+TG+CT+CT+T+C+C+GA+TC |
| NO: 12 | |
| SEQ ID | A+CA+CT+CTT+T+CC+CTA+CA |
| NO: 13 | |
| SEQ ID | +C+GA+CG+CT+CTT+CCG+ATC |
| NO: 14 | |
| SEQ ID | A+T+G+T+G+TA+TA+A+G+A+GA+C+AG |
| NO: 23 | |
| SEQ ID | T+CG+T+CGG+C+AG+CG+T+C+AG |
| NO: 24 | |
| SEQ ID | G+T+CT+CG+T+G+G G+CT+CG+GAG |
| NO: 25 | |

The hybridization blocker set shown in Table II includes with reference to FIGS. 2 and 3:

a first hybridization blocker, “F”, comprising an oligonucleotide sequence which is the complement of the first extension primer-binding site (exemplified by SEQ ID NO:2);

a second hybridization blocker, “E”, comprising the oligonucleotide sequence of the first sequencing primer-binding site (exemplified by SEQ ID NO:6);

a third hybridization blocker, “D”, comprising the oligonucleotide sequence of the first transposon end (exemplified by SEQ ID NO:11);

a fourth hybridization blocker, “C”, comprising an oligonucleotide sequence which is the complement of the second sequencing primer-binding site (exemplified by SEQ ID NO:12);

a fifth hybridization blocker, “A”, comprising the oligonucleotide sequence of the second extension primer-binding site (exemplified by SEQ ID NO:13);

a sixth hybridization blocker, “B”, comprising the oligonucleotide sequence of portion A of the third sequencing primer-binding site (exemplified by SEQ ID NO:14);

a seventh hybridization blocker, “H”, comprising the oligonucleotide sequence of portion B of the third sequencing primer-binding site (exemplified by SEQ ID NO:23);

an eighth hybridization blocker, “G”, comprising an oligonucleotide which is the complement of portion A of the fourth sequencing primer-binding site (exemplified by SEQ ID NO:24); and

a ninth hybridization blocker, “I”, comprising an oligonucleotide which is the complement of portion B of the fourth sequencing primer-binding site (exemplified by SEQ ID NO:25).

Methods of massively parallel sequencing according to aspects of the present disclosure include hybridizing a plurality of nucleic acid sequencing library molecules with a set of hybridization blockers, wherein the set of hybridization blockers efficiently blocks interactions between the adapter regions of different library molecules and is therefore effective to reduce the capture of non-target sequences during a hybridization capture enrichment step, to maximize the efficiency of the massively parallel sequencing techniques.

Methods of generating a sequencing library are provided according to aspects of the present disclosure which include generating nucleic acid sequencing library molecules which include a template strand and a complementary strand, the template strand including an insert disposed between a first adapter and a second adapter and the complementary strand includes a complement of the insert disposed between a complement of the first adapter and a complement of the second adapter.

According to aspects of the present disclosure, generating nucleic acid sequencing library molecules includes providing a plurality of inserts; covalently bonding a first adapter to the 5′ end of each insert, and covalently bonding a second adapter to the 3′ end of each insert, producing nucleic acid sequencing library molecules. Covalently bonding the adapters to the inserts may be accomplished by ligation using a ligase enzyme.

Sequencing library molecules optionally contain nucleic acid sequences present in one or more of the adapters and not present in the inserts. Such adapter sequences may add functionality such as primer binding sites for extension, primer binding sites for amplification reactions and/or a functional group for chemical bonding to a substrate. Non-limiting examples of nucleic acid sequences included in adapters that can be incorporated into sequencing library molecules, and thereby incorporated into the library produced, include, universal sequences, index sequences, identification sequences, detection sequences, sorting sequences, captures sequences, restriction enzyme cleavage sites, sequencing primer binding site sequences, extension primer binding site sequences, and amplification primer binding site sequences.

The term “universal sequence” refers to a nucleic acid sequence that is present in a plurality of nucleic acid molecules that also contain nucleic acid sequences which are not common to the plurality of nucleic acid molecules. A universal sequence allows the plurality of nucleic acid molecules to share a common functional aspect, such as binding to a particular primer or capture moiety. Non-limiting examples of universal extension primer binding sites include sequences that are identical to or complementary to P5 and P7 primers. P5 and P7 primers, their complements, and uses, such as in flow cells for capture on a flow cells substrate for next generation sequencing (NGS), are known in the art, for example as detailed in WO2015106941.

According to aspects of methods of massively parallel nucleic acid sequencing of the present disclosure, a set of hybridization blockers of the present disclosure is annealed to nucleic acid sequencing library molecules to reduce interactions between the nucleic acid sequencing library molecules which would otherwise interfere with massively parallel nucleic acid sequencing, reducing the quality of the sequencing output.

Hybridization of a set of hybridization blockers to nucleic acid sequencing library molecules is performed at an hybridization temperature. The hybridization temperature depends on factors, including the nucleic acid sequence of the hybridization blockers, and the nucleic acid sequences of the sequencing library molecules to which the hybridization blockers anneals, and the composition of the hybridization medium, including factors such as salt concentration, and concentration of additives, such as but not limited to, a surfactant, formamide, betaine, polyethylene glycol, SDS, and DMSO. Typically, the hybridization temperature is in the range of 30° C. to 65° C., but can be higher or lower. The hybridization temperature may be higher or lower than the melting temperature (Tm) of the hybridization blockers and the corresponding portion of the sequencing library molecules. Often hybridization temperatures are higher than the Tm of the hybridization blockers to improve the stringency of the reaction. A hybridization step is performed for a suitable period of time, typically in the range of 5 minutes to 48 hours, such as in the range of 10 minutes to 24 hours, and such as in the range of 15 minutes to 48 overnight.

Following hybridization of the set of hybridization blockers to nucleic acid sequencing library molecules, one or more high stringency washes may be performed. One or more optional low stringency washes may be performed prior to the one or more high stringency washes.

A step of capture hybridization may be performed following hybridization of the set of hybridization blockers to nucleic acid sequencing library molecules. Capture hybridization provides for enrichment of nucleic acid sequencing library molecules containing particular nucleic acid sequences which hybridize to probes, thereby capturing the sequencing library molecules. The probes may be attached to a support, such as a flow cell, or particle.

Nucleic acid sequencing may then be performed on the captured sequencing library molecules, advantageously reducing sequencing of non-desired nucleic acid sequences.

Sequencing of sequencing library molecules is accomplished using any of various sequencing methodologies, particularly, massively parallel sequencing methodologies (“next generation sequencing”).

Methods of massively parallel sequencing of the present disclosure are not limited to use with specific equipment and associated procedures. Rather, the compositions and methods of the present disclosure may be used in any of various sequencing protocols and with various sequencing equipment.

Reverse transcribing target RNA, producing first strand complementary DNA (cDNA), is accomplished using a reverse transcriptase enzyme under reaction conditions compatible with reverse transcriptase enzyme activity to transcribe the target RNA. Particular conditions and protocols for reverse transcription are detailed in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002.

Methods of generating a sequencing library are provided according to the present disclosure which further include polymerizing a second strand of DNA complementary to the first strand cDNA, producing double-stranded cDNA. Polymerizing a second strand of DNA complementary to the first strand cDNA may include providing an appropriate DNA polymerase and polymerizing to produce double-stranded cDNA under reaction conditions compatible with DNA polymerase activity to produce double-stranded cDNA, i.e. insert.

The term “extension primer” as used herein refers to an oligonucleotide capable of acting as a point of initiation of enzymatic synthesis of an oligonucleotide primer extension product under conditions in which synthesis of an oligonucleotide primer extension product which is complementary to a target nucleic acid is induced. Such conditions include the presence of nucleotides and a suitable polymerase, at a suitable temperature and pH.

Primer extension, producing a first strand complementary DNA, is accomplished using a polymerase enzyme under extension reaction conditions compatible with the polymerase activity to produce a complementary strand. Particular conditions and protocols for primer extension are detailed in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002.

Suitable polymerases may include bacterial DNA polymerases, eukaryotic DNA polymerases, archaeal DNA polymerases, viral DNA polymerases, Taq polymerase, DNA polymerase I, T4 DNA polymerase, Pfu polymerase, and phage DNA polymerases, including the Klenow fragment of DNA polymerase I, SEQUENASE 1.0 and SEQUENASE 2.0 (U.S. Biochemical), T5 DNA polymerase, and Phi29 DNA polymerase, among others.

According to further aspects, methods of generating a sequencing library include amplifying the sequencing library molecules. Amplification of the sequencing library molecules may be performed before a hybridization capture step, after a hybridization capture step, or both before and after a hybridization capture step.

The terms “amplify, “amplification,” and “amplifying” are used to refer generally to a process of copying a nucleic acid molecule, or portion thereof, to produce at least one copy of the nucleic acid, or portion thereof.

Amplification of template DNA is achieved using an in vitro amplification method. The terms “amplify, “amplification,” and “amplifying” are used to refer generally to a method or technique for copying a template nucleic acid, thereby producing nucleic acids including copies of all or a portion of the template nucleic acid, the produced nucleic acids also termed amplicons.

Amplification methods illustratively including PCR, ligation-mediated PCR (LM-PCR), phi-29 PCR, and other nucleic acid amplification methods, for instance, as described in C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

Amplicons optionally contain nucleic acid sequences present in the primers and not present in the original DNA template. Such primer-derived nucleic acids add functionality such as primer binding sites for additional amplification reactions and/or a functional group for chemical bonding to a substrate. Non-limiting examples of primer-derived nucleic acid sequences that can be incorporated into amplicons, and thereby incorporated into the library produced, include, universal sequences, adapters, index sequences, identification sequences, detection sequences, sorting sequences, captures sequences, restriction enzyme cleavage sites, extension primer binding site sequences, sequencing primer binding site sequences, and amplification primer binding site sequences.

Kits are provided according to aspects of the disclosure for use in massively parallel sequencing of a plurality of nucleic acid sequencing library molecules, wherein the kits include a set of hybridization blockers as described or shown herein. Such kits may further include instructional material for use of the set of hybridization blockers in methods of massively parallel sequencing. One or more ancillary reagents such as PCR primers, buffers, enzymes, paramagnetic beads, sequencing primers, washing solutions, hybridization solutions, detectable labels, detection reagents and the like are also optionally included.

Embodiments of inventive compositions and methods are illustrated in the following examples. These examples are provided for illustrative purposes and are not considered limitations on the scope of inventive compositions and methods.

### EXAMPLES

### Example 1

Experimental Conditions:

TruSeq & Nextera libraries were generated and added in same Hybridization (Hyb) mixture in an amount of 250 ng each (half the normal amount of 500 ng each). Hyb used water (None), or PE Universal blockers (UB). For the hybridization reaction, a set of hybridization blocking oligonucleotides of the present disclosure, PE Universal blockers, was first prepared by adding each of the nine hybridization blockers to a hybridization reaction vessel in equimolar amounts. The PE Universal blockers used in this example all included at least one Tₘ increasing nucleotide and were, with reference to FIGS. 2 and 3:

a first blocker comprising an oligonucleotide sequence which is the complement of the first extension primer-binding site;

a second blocker comprising the oligonucleotide sequence of the first sequencing primer-binding site;

a third blocker comprising the oligonucleotide sequence of the first transposon end;

a fourth blocker comprising an oligonucleotide sequence which is the complement of the second sequencing primer-binding site;

a fifth blocker comprising the oligonucleotide sequence of the second extension primer-binding site;

a sixth blocker comprising the oligonucleotide sequence of portion A of the third sequencing primer-binding site;

a seventh blocker comprising the oligonucleotide sequence of portion B of the third sequencing primer-binding site;

an eighth blocker comprising an oligonucleotide which is the complement of portion A of the fourth sequencing primer-binding site; and

a ninth blocker comprising an oligonucleotide which is the complement of portion B of the fourth sequencing primer-binding site.

This is combined with the indicated amounts of library in appropriate hybridization buffer, including human Cot DNA, and incubated at the desired temperature overnight. Following hybridization, a first wash is performed, followed by two stringent washes. Capture hybridization, and post-capture PCR can be performed, resulting in post-capture PCR fragments, followed by sequencing.

All post Hyb reactions amplified using IDT xGen Library Amplification Primers. Libraries were analyzed using Qubit dsDNA HS and BioAnalyzer. Libraries were pooled in equal concentrations and sequenced on a MiSeq v3 300 cycle (2×150 bp).

Data Analysis:

Libraries were aligned to the human genome using bowtie2 and Samtools. On-target bases were determined using Picard CollectHSMetrics. On-target defined as bases overlapping a target region ±250 bp.

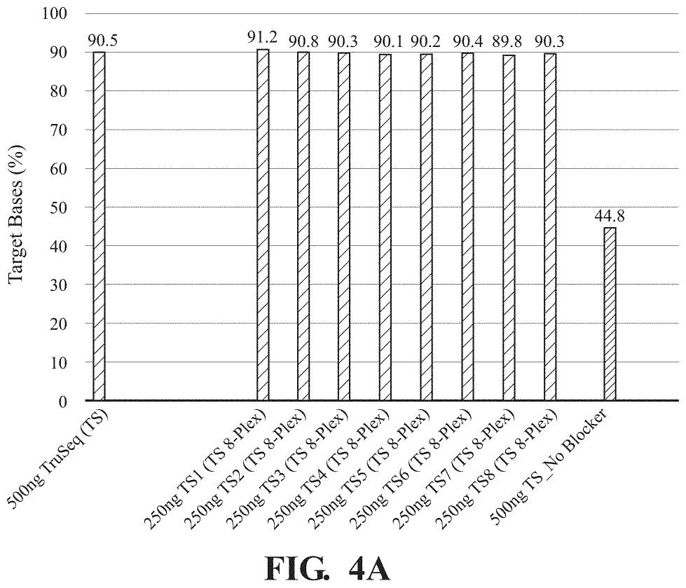

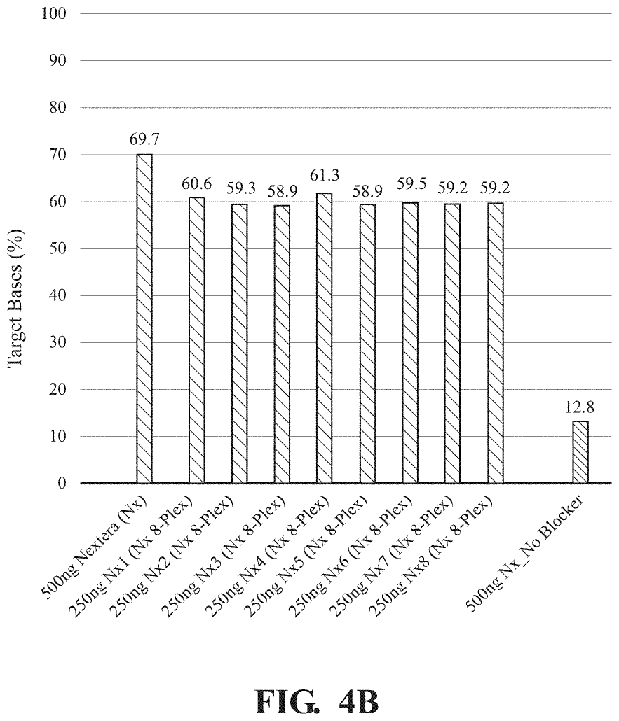

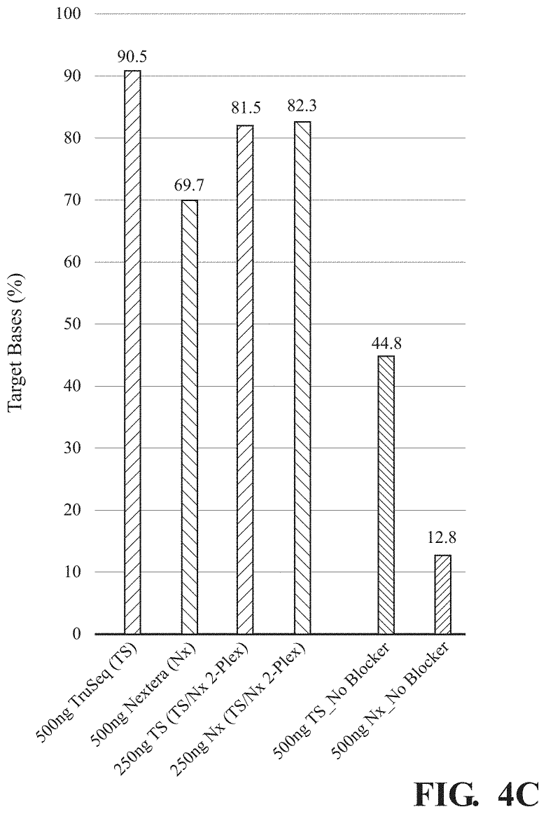

Results are shown in FIGS. 4A, 4B, and 4C.

FIG. 4A is a graph showing effects of an oligonucleotide hybridization blocker set composition according to aspects of the present disclosure including nine oligonucleotide hybridization blockers described in this example on the percentage of on-target bases when the oligonucleotide hybridization blocker set composition is included in the hybridization mix with one single plex TruSeq (TS) library at 500 ng or up to eight TruSeq libraries (each at 250 ng, for an 8-plex total of 2 micrograms in a single hybridization), compared to without any oligonucleotide hybridization blockers; as shown, the nine oligonucleotide hybridization blocker set described in this example blocks as effectively for the single plex TruSeq library when compared with either of: (i) any of the individual libraries of the 8-plex (each at 250 ng); or, (ii) the combined libraries (e.g., average) of the 8-plex totaling 2 micrograms. Those of ordinary skill will understand that the disclosed methods and compositions are not limited to an 8-plex, with FIG. 4A being merely illustrative of one embodiment, and the methods are compositions are applicable to greater numbers of libraries, including 10-plex, 16-plex, etc.

FIG. 4B is a graph showing effects of an oligonucleotide hybridization blocker set composition according to aspects of the present disclosure including nine oligonucleotide hybridization blockers described in this example on the percentage of on-target bases when the oligonucleotide hybridization blocker set composition is included in the hybridization mix with one single plex Nextera (Nx) library (500 ng) or up to eight Nextera libraries (each at 250 ng, for an 8-plex total of 2 micrograms in a single hybridization), compared to without any oligonucleotide hybridization blockers; as shown, the nine oligonucleotide hybridization blocker mix described in this example blocks as effectively for the single plex Nextera library when compared with either of: (i) any of the individual libraries of the 8-plex (each at 250 ng); or, (ii) the combined libraries (e.g., average) of the 8-plex totaling 2 micrograms. Those of ordinary skill will understand that the disclosed methods and compositions are not limited to an 8-plex, with FIG. 4B being merely illustrative of one embodiment, and the methods are compositions are applicable to greater numbers of libraries, including 10-plex, 16-plex, etc.

FIG. 4C is a graph showing effects of an oligonucleotide hybridization blocker set composition according to aspects of the present disclosure including nine oligonucleotide hybridization blockers described in this example on the percentage of on-target bases when the oligonucleotide hybridization blocker set composition is included in the hybridization mix of three different types of libraries: (i) a single plex Nextera (Nx) library (at 500 ng); (ii) a single plex TruSeq (TS) library (also at 500 ng); and, (iii) a duplexed TruSeq library with a Nextera library (each at 250 ng), in the same hybridization mix, compared to a single plex TruSeq (500 ng) and a single plex Nextera (500 ng) hybridization mix without any oligonucleotide hybridization blockers; as shown, the nine oligonucleotide hybridization blocker set described in this example blocks approximately equally for the duplexed TruSeq and Nextera libraries at 250 ng each when duplexed in the same hybridization mix, and when compared to the individual single plex TruSeq and Nextera libraries at 500 ng each, block better than the Nextera library at 500 ng and only slightly less than the TruSeq library at 500 ng. As with the other illustrative embodiments of the other Figures, FIG. 4C is not limited to the embodiment shown.

### Example 2

Experimental Conditions:

TruSeq libraries were generated and added in same Hyb mixture in an amount of 500 ng library input for single-plex, and 250 ng each library input for 8-plex for a total of 2 ug library input. Hyb used 24, of IDT xGen TS-Mix (IDT), water (None), or PE Universal blockers (UB). The PE Universal blockers used in this example all included at least one Tm increasing nucleotide and were, with reference to FIGS. 2 and 3:

a first blocker comprising an oligonucleotide sequence which is the complement of the first extension primer-binding site;

a second blocker comprising the oligonucleotide sequence of the first sequencing primer-binding site;

a third blocker comprising the oligonucleotide sequence of the first transposon end;

a fourth blocker comprising an oligonucleotide sequence which is the complement of the second sequencing primer-binding site;

a fifth blocker comprising the oligonucleotide sequence of the second extension primer-binding site;

a sixth blocker comprising the oligonucleotide sequence of portion A of the third sequencing primer-binding site;

a seventh blocker comprising the oligonucleotide sequence of portion B of the third sequencing primer-binding site;

an eighth blocker comprising an oligonucleotide which is the complement of portion A of the fourth sequencing primer-binding site; and

a ninth blocker comprising an oligonucleotide which is the complement of portion B of the fourth sequencing primer-binding site.

The Hyb reaction was performed overnight. All post-Hyb reactions amplified using IDT xGen Library Amplification Primers. Libraries were analyzed using Qubit dsDNA HS and BioAnalyzer. Libraries were pooled in equal concentrations and sequenced on a MiSeq v3 300 cycle (2×150 bp).

Data Analysis:

Libraries were aligned to the human genome using bowtie2 and Samtools. On-target bases were determined using Picard CollectHSMetrics. On-target defined as bases overlapping a target region 250 bp.

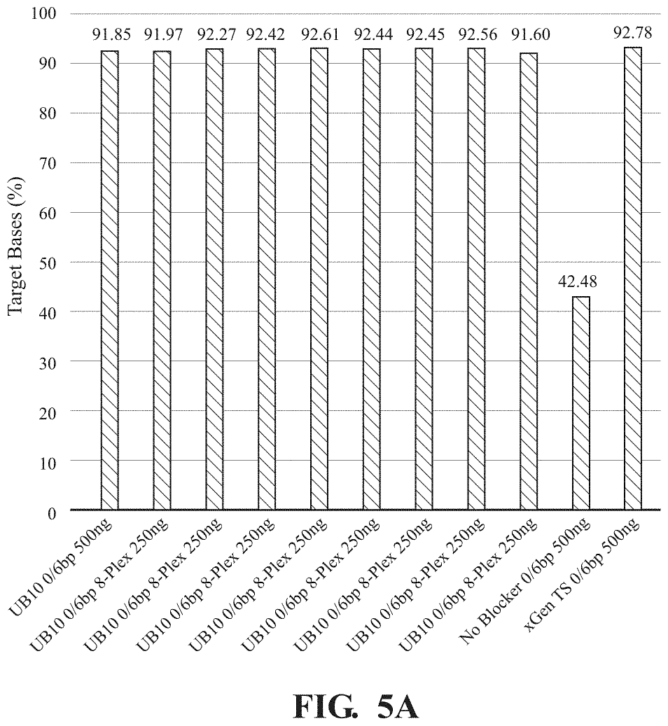

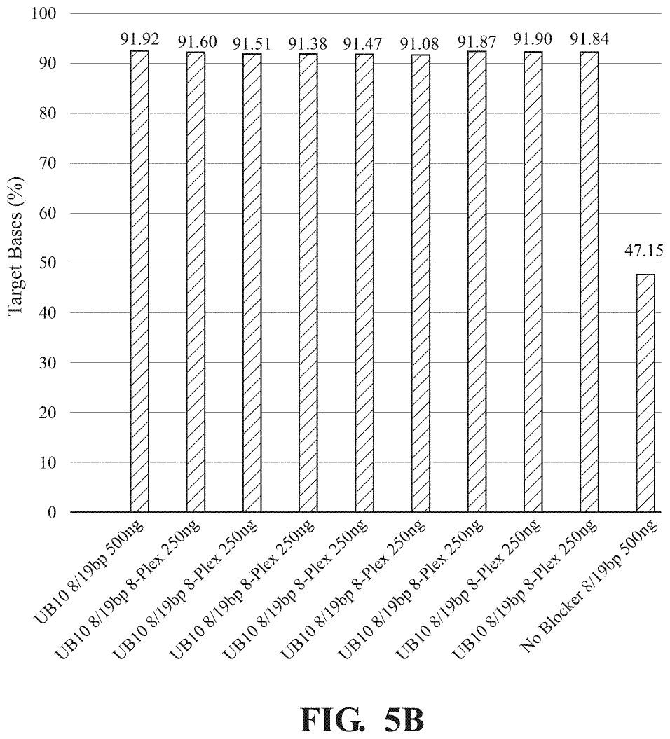

Results are shown in FIGS. 5A and 5B.

FIG. 5A is a graph showing effects of an oligonucleotide hybridization blocker composition including nine oligonucleotide hybridization blockers described in this example on the percentage of on-target bases when the oligonucleotide hybridization blocker set composition is included in the hybridization mix with one single-plex TruSeq (TS) library (at 500 ng), or up to eight TruSeq (TS) libraries (each at 250 ng, or an 8-plex of 2 micrograms in a single hybridization) compared to without any oligonucleotide hybridization blockers, where the TruSeq libraries include TruSeq adapters with no separation or 6 bp separation (0/6 bp) separation between P5/R1 and P7/R2 sequence regions, respectively; the nine oligonucleotide hybridization blocker mix described in this example blocks TruSeq adapters independently of index/UMI length.

FIG. 5B is a graph showing effects of an oligonucleotide hybridization blocker set composition including nine oligonucleotide hybridization blockers described in this example on the percentage of on-target bases when the oligonucleotide hybridization blocker composition is included in the hybridization mix with one single plex TruSeq (TS) library (500 ng) or up to eight TruSeq libraries (each at 250 ng, or an 8-plex of 2 micrograms in a single hybridization) compared to without any blockers where the TruSeq libraries include TruSeq adapters with 8 bp separation or 19 bp separation (8/19 bp) separation between P5/R1 and P7/R2 sequence regions, respectively; thus, the nine oligonucleotide hybridization blocker set describe in this example blocks TruSeq adapters independently of index/UMI length.

### Example 3

Experimental Conditions:

500, 750, 1000, 1500, 2000*, and 2500 ng of NEXTflex prepared library from NA12878 (*8-plex of 250 ng each library). Hyb with IDT Exome Research Panel v2.

IDT xGen hyb/wash system was then used. The same amount of an oligonucleotide hybridization blocker set including 9 oligonucleotide hybridization blockers (or no blockers) was used. The 9 oligonucleotide hybridization blockers used in this example all included at least one Tm increasing nucleotide and were, with reference to FIGS. 2 and 3:

a first blocker comprising an oligonucleotide sequence which is the complement of the first extension primer-binding site;

a second blocker comprising the oligonucleotide sequence of the first sequencing primer-binding site;

a third blocker comprising the oligonucleotide sequence of the first transposon end;

a fourth blocker comprising an oligonucleotide sequence which is the complement of the second sequencing primer-binding site;

a fifth blocker comprising the oligonucleotide sequence of the second extension primer-binding site;

a sixth blocker comprising the oligonucleotide sequence of portion A of the third sequencing primer-binding site;

a seventh blocker comprising the oligonucleotide sequence of portion B of the third sequencing primer-binding site;

an eighth blocker comprising an oligonucleotide which is the complement of portion A of the fourth sequencing primer-binding site; and

a ninth blocker comprising an oligonucleotide which is the complement of portion B of the fourth sequencing primer-binding site.

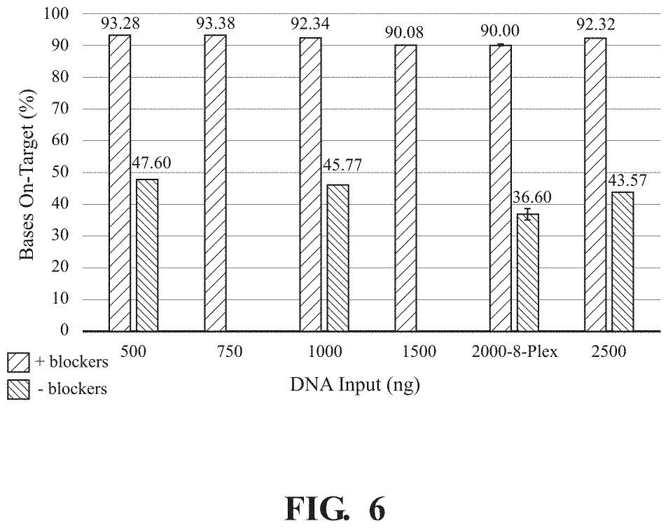

Results are shown in FIG. 6.

FIG. 6 is a graph showing effects of an oligonucleotide hybridization blocker set composition including nine oligonucleotide hybridization blockers described in this example on the percentage of on-target bases when the oligonucleotide hybridization blocker composition is included in the hybridization mix with 8-plex of NEXTFLEX libraries (250 ng of each library in the 8-plex mix) in varying amounts in the range of 500-2500 ng. The results show that the oligonucleotide hybridization blocker set composition can be used in capture hybridization mixtures with up to 2500 ng library DNA without loss of performance.

Items

Item 1. A kit for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, the kit comprising: a set of hybridization blockers, each hybridization blocker comprising an oligonucleotide having a nucleic acid sequence which is a complement of an oligonucleotide present in the template strand and/or complementary strand of at least some of the plurality of nucleic acid sequencing library molecules, with the proviso that none of the hybridization blockers comprises an oligonucleotide having a nucleic acid sequence which is a complement of the insert sequence, or an index sequence, if present, of the nucleic acid sequencing library molecules, each oligonucleotide of each hybridization blocker comprising a plurality of Tₘ increasing nucleotide analogs, wherein the set of hybridization blockers comprises at least two pairs of split hybridization blockers, each pair of split hybridization blockers comprising a first split oligonucleotide having an oligonucleotide sequence complementary to a portion A of a target oligonucleotide included in the template strand or the complementary strand and a second split oligonucleotide having an oligonucleotide sequence complementary to a portion B of the target oligonucleotide included in the template strand or the complementary strand, wherein portion A and portion B are adjacent in the target oligonucleotide, and wherein the portion A and the portion B of the target oligonucleotide to which each pair of split hybridization blockers hybridizes, is different.

Item 2. The kit of item 1, wherein the first adapter comprises a first extension primer-binding site and a first sequencing primer-binding site, the second adapter comprises a second extension primer-binding site and a second sequencing primer-binding site, wherein the complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the first pair of split hybridization blockers comprises a first split oligonucleotide having an oligonucleotide sequence complementary to a portion A of the first sequencing primer-binding site and a second split oligonucleotide having an oligonucleotide sequence complementary to a portion B of the first sequencing primer-binding site, or wherein the first pair of split hybridization blockers comprises a first split oligonucleotide having an oligonucleotide sequence complementary to a portion A of the complement of the first sequencing primer-binding site and a second split oligonucleotide having an oligonucleotide sequence complementary to a portion B of the complement of the first sequencing primer-binding site.

Item 3. The kit of item 1 or 2, wherein the first adapter comprises a first extension primer-binding site and a first sequencing primer-binding site, the second adapter comprises a second extension primer-binding site and a second sequencing primer-binding site, wherein the complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the second pair of split hybridization blockers comprises a first split oligonucleotide having an oligonucleotide sequence complementary to a portion A of the second sequencing primer-binding site and a second split oligonucleotide having an oligonucleotide sequence complementary to a portion B of the second sequencing primer-binding site, or wherein the second pair of split hybridization blockers comprises a first split oligonucleotide having an oligonucleotide sequence complementary to a portion A of the complement of the second sequencing primer-binding site and a second split oligonucleotide having an oligonucleotide sequence complementary to a portion B of the complement of the second sequencing primer-binding site.

Item 4. The kit of item 1, wherein the insert of the template strand is a transposon insert disposed between a first transposon end and a second transposon end, the first transposon end adjacent the first sequencing primer-binding site and the second transposon end adjacent the second sequencing primer-binding site, the complementary strand comprising a complement of the transposon insert, the first transposon end and the second transposon end.

Item 5. The kit of item 4, wherein the first adapter comprises a first extension primer-binding site and a first sequencing primer-binding site, the second adapter comprises a second extension primer-binding site and a second sequencing primer-binding site, wherein complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the first pair of split hybridization blockers comprises a first split oligonucleotide having an oligonucleotide sequence complementary to at least a portion of the first sequencing primer-binding site and a second split oligonucleotide having an oligonucleotide sequence complementary to at least a portion of the first transposon end, or wherein the first pair of split hybridization blockers comprises a first split oligonucleotide having an oligonucleotide sequence complementary to at least a portion of the complement of the first sequencing primer-binding site and a second split oligonucleotide having an oligonucleotide sequence complementary to at least a portion of the complement of the first transposon end.

Item 6. The kit of item 4 or 5, wherein the first adapter comprises a first extension primer-binding site and a first sequencing primer-binding site, the second adapter comprises a second extension primer-binding site and a second sequencing primer-binding site, wherein complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the second pair of split hybridization blockers comprises a first split oligonucleotide having an oligonucleotide sequence complementary to at least a portion of the second sequencing primer-binding site and a second split oligonucleotide having an oligonucleotide sequence complementary to at least a portion of the second transposon end, or wherein the second pair of split hybridization blockers comprises a first split oligonucleotide having an oligonucleotide sequence complementary to at least a portion of the complement of the second sequencing primer-binding site and a second split oligonucleotide having an oligonucleotide sequence complementary to at least a portion of the complement of the second transposon end.

Item 7. The kit according to any of items 1 to 6, wherein the hybridization blocker set further comprises an oligonucleotide hybridization blocker comprising a nucleic acid sequence which is a complement of the first extension primer or an oligonucleotide hybridization blocker comprising the nucleic acid sequence of the first extension primer.

Item 8. The kit according to any of items 1 to 7, wherein the hybridization blocker set further comprises an oligonucleotide hybridization blocker comprising a nucleic acid sequence which is a complement of the second extension primer or an oligonucleotide hybridization blocker comprising the nucleic acid sequence of the second extension primer.

Item 9. The kit according to any of items 1 to 8, for use in massively parallel sequencing of a plurality of nucleic acid sequencing library molecules, wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers: a first hybridization blocker comprising an oligonucleotide sequence which comprises at least a portion of a complement of the first extension primer-binding site; a second hybridization blocker comprising at least a portion of the oligonucleotide sequence of the first sequencing primer-binding site; a third hybridization blocker comprising at least a portion of the oligonucleotide sequence of the first transposon end; a fourth hybridization blocker comprising an oligonucleotide sequence which is a complement of at least a portion of the second sequencing primer-binding site; a fifth hybridization blocker comprising at least a portion of the oligonucleotide sequence of the second extension primer-binding site; a sixth hybridization blocker comprising at least a portion of the oligonucleotide sequence of portion A of the third sequencing primer-binding site; a seventh hybridization blocker comprising the oligonucleotide sequence of portion B of the third sequencing primer-binding site; an eighth hybridization blocker comprising an oligonucleotide which is the complement of portion A of the fourth sequencing primer-binding site; and a ninth hybridization blocker comprising an oligonucleotide which is the complement of portion B of the fourth sequencing primer-binding site.

Item 10. The kit according to any of items 1 to 8, for use in massively parallel sequencing of a plurality of nucleic acid sequencing library molecules, wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers: a first hybridization blocker comprising an oligonucleotide sequence which hybridizes to the complement of the first extension primer-binding site; a second hybridization blocker comprising the oligonucleotide sequence which is the complement of the first sequencing primer-binding site; a third hybridization blocker comprising the oligonucleotide sequence hybridizes to the first transposon end; a fourth hybridization blocker comprising an oligonucleotide sequence which hybridizes to the complement of the second sequencing primer-binding site; a fifth hybridization blocker comprising the oligonucleotide sequence which is the complement of the second extension primer-binding site; a sixth hybridization blocker comprising an oligonucleotide sequence which hybridizes to portion A of the third sequencing primer-binding site; a seventh hybridization blocker comprising an oligonucleotide sequence which hybridizes to portion B of the third sequencing primer-binding site; an eighth hybridization blocker comprising an oligonucleotide which hybridizes to the complement of portion A of the fourth sequencing primer-binding site; and a ninth hybridization blocker comprising an oligonucleotide which hybridizes to the complement of portion B of the fourth sequencing primer-binding site.

Item 11. A kit for use in massively parallel sequencing of a plurality of nucleic acid sequence library inserts of at least four types of nucleic acid sequencing library molecule, the first type of nucleic acid sequencing library molecule comprising, a nucleic acid transposon insert, the transposon insert disposed between, and bonded to, a first adapter and a second adapter, the first adapter comprising at least a first extension primer-binding oligonucleotide, a first sequencing primer-binding oligonucleotide, and a first transposon end sequence, the second adapter comprising at least a second extension primer-binding oligonucleotide, a second sequencing primer-binding oligonucleotide, and a second transposon end sequence, wherein at least one of the first adapter and the second adapter comprises an index oligonucleotide, the second type of nucleic acid sequencing library molecule comprising a complement nucleic acid sequence of the first type of nucleic acid sequencing library molecule wherein the complement nucleic acid sequence of the first type of nucleic acid sequencing library molecule comprises a complement nucleic acid transposon insert, the complement transposon insert disposed between, and bonded to, a complement first adapter and a complement second adapter, the complement first adapter comprising at least a complement first extension primer-binding oligonucleotide, a complement first sequencing primer-binding oligonucleotide, and a complement first transposon end sequence, the complement second adapter comprising at least a complement second extension primer-binding oligonucleotide, a complement second sequencing primer-binding oligonucleotide, and a complement second transposon end sequence, wherein at least one of the complement first adapter and the complement second adapter comprises a complement index oligonucleotide, the third type of nucleic acid sequencing library molecule comprising a nucleic acid insert, each nucleic acid insert disposed between, and bonded to, a third adapter and a fourth adapter, the third adapter comprising at least a third extension primer-binding oligonucleotide, and a third sequencing primer-binding oligonucleotide, the fourth adapter comprising at least a fourth extension primer-binding oligonucleotide, and a fourth sequencing primer-binding oligonucleotide, with the proviso that neither the third adapter nor the fourth adapter comprises a transposon end sequence, wherein at least one of the third adapter and the fourth adapter comprises an index oligonucleotide; the fourth type of nucleic acid sequencing library molecule comprising a complement nucleic acid sequence of the third type of nucleic acid sequencing library molecule wherein the complement nucleic acid sequence of the third type of nucleic acid sequencing library molecule comprises a complement nucleic acid insert, the complement nucleic acid insert disposed between, and bonded to, a complement third adapter and a complement fourth adapter, the complement third adapter comprising at least a complement third extension primer-binding oligonucleotide, and a complement third sequencing primer-binding oligonucleotide, the complement fourth adapter comprising at least a complement fourth extension primer-binding oligonucleotide, and a complement fourth sequencing primer-binding oligonucleotide, with the proviso that neither the complement third adapter nor the complement fourth adapter comprises a transposon end sequence, wherein at least one of the complement third adapter and the complement fourth adapter comprises a complement index oligonucleotide; the kit comprising at least nine blocking oligonucleotides, wherein each of the at least nine blocking oligonucleotides comprises at least one Tₘ increasing nucleotide, the at least nine blocking oligonucleotides comprising: a first hybridization blocker comprising an oligonucleotide sequence which is the complement of the first extension primer-binding site; a second hybridization blocker comprising the oligonucleotide sequence of the first sequencing primer-binding site; a third hybridization blocker comprising the oligonucleotide sequence of the first transposon end; a fourth hybridization blocker comprising an oligonucleotide sequence which is the complement of the second sequencing primer-binding site; a fifth hybridization blocker comprising the oligonucleotide sequence of the second extension primer-binding site; a sixth hybridization blocker comprising the oligonucleotide sequence of portion A of the third sequencing primer-binding site; a seventh hybridization blocker comprising the oligonucleotide sequence of portion B of the third sequencing primer-binding site; an eighth hybridization blocker comprising an oligonucleotide which is the complement of portion A of the fourth sequencing primer-binding site; and a ninth hybridization blocker comprising an oligonucleotide which is the complement of portion B of the fourth sequencing primer-binding site.

Item 12. The kit of item 11, for use in massively parallel sequencing of a plurality of nucleic acid sequence library inserts of at least four types of nucleic acid sequencing library molecule, comprising: a first hybridization blocker comprising an oligonucleotide sequence which hybridizes to the complement of the first extension primer-binding site; a second hybridization blocker comprising the oligonucleotide sequence which is the complement of the first sequencing primer-binding site; a third hybridization blocker comprising the oligonucleotide sequence hybridizes to the first transposon end; a fourth hybridization blocker comprising an oligonucleotide sequence which hybridizes to the complement of the second sequencing primer-binding site; a fifth hybridization blocker comprising the oligonucleotide sequence which is the complement of the second extension primer-binding site; a sixth hybridization blocker comprising an oligonucleotide sequence which hybridizes to portion A of the third sequencing primer-binding site; a seventh hybridization blocker comprising an oligonucleotide sequence which hybridizes to portion B of the third sequencing primer-binding site; an eighth hybridization blocker comprising an oligonucleotide which hybridizes to the complement of portion A of the fourth sequencing primer-binding site; and a ninth hybridization blocker comprising an oligonucleotide which hybridizes to the complement of portion B of the fourth sequencing primer-binding site.

Item 13. A kit for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the insert having a first end and a second end, the first adapter comprising a first primer-binding site and a first target oligonucleotide, the first adapter optionally comprising a first index disposed between the first primer-binding site and the first target oligonucleotide, the first target oligonucleotide disposed between the first index, if present, and the first end of the insert, or the first target oligonucleotide disposed between the first primer-binding site and the first insert end, the second adapter comprising a second target oligonucleotide and a second primer-binding site, the second adapter optionally comprising a second index disposed between the second target oligonucleotide and the second primer-binding site, the second target oligonucleotide disposed between the second end of the insert and the second index, if present, or the second target oligonucleotide disposed between the second target nucleotide and the second primer-binding site, wherein the template strand comprises at least the first or the second index, the complementary strand being the complement of the template strand, the kit comprising: a set of hybridization blockers, each hybridization blocker comprising an oligonucleotide having a nucleic acid sequence which can hybridize to one of: the first target oligonucleotide, the second target oligonucleotide, the complement of the first target oligonucleotide, or the complement of the second target oligonucleotide, each oligonucleotide of each hybridization blocker comprising a plurality of Tₘ increasing nucleotide analogs, wherein the set of hybridization blockers comprises a first set of at least two hybridization blockers that comprise a first pair of split hybridization blockers, each pair of split hybridization blockers comprising a first split oligonucleotide having an oligonucleotide sequence complementary to one of: a portion A of either the first target oligonucleotide or the complement of portion A in the complementary strand, and a second split oligonucleotide having an oligonucleotide sequence complementary to one of: a portion B of the first target oligonucleotide or the complement of portion B in the complementary strand, wherein the first split oligonucleotide and the second split oligonucleotide each hybridize to either the portion A and the portion B on the template strand, or the complement of the portion A and the complement of the portion B on the complementary strand, and wherein the portion A and the portion B of the first target oligonucleotide are different, wherein the set of hybridization blockers further comprises a second pair of split hybridization blockers, the second pair comprising a first split oligonucleotide having an oligonucleotide sequence complementary to one of: a portion C of either the second target oligonucleotide or the complement of portion C in the complementary strand, and a second split oligonucleotide having an oligonucleotide sequence complementary to one of: a portion D of the second target oligonucleotide or the complement of portion D in the complementary strand, wherein the first split oligonucleotide and the second split oligonucleotide of the second pair each hybridize to either the portion C and the portion D on the template strand, or the complement of the portion C and the complement of the portion D on the complementary strand, and wherein the portion C and the portion D of the second target oligonucleotide are different, and, wherein, if the first pair of split hybridization blockers hybridizes to the portion A and the portion B of the template strand, then the second pair of split hybridization blockers hybridizes to the complement of the portion C and the complement of the portion D of the complementary strand, and, if the first pair of split hybridization blockers hybridizes to the complement of the portion A and the complement of the portion B of the complementary strand, then the second pair of split hybridization blockers hybridizes to the portion C and the portion D of the template strand.

Item 14. The kit of item 13, wherein the portion A and the portion B of the first target oligonucleotide are adjacent.

Item 15. The kit of item 13 or 14, wherein the portion C and the portion D of the second target oligonucleotide are adjacent.

Item 16. A set of hybridization blockers, each of the hybridization blockers comprising at least one Tm increasing nucleotide, the set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, the set of hybridization blockers comprising: a first pair of split hybridization blockers, comprising a first split oligonucleotide having an oligonucleotide sequence complementary to a portion A of a first target oligonucleotide included in the template strand or the complementary strand and a second split oligonucleotide having an oligonucleotide sequence complementary to a portion B of the first target oligonucleotide included in the template strand or the complementary strand, wherein portion A and portion B are adjacent in the target oligonucleotide, such that the first pair of split hybridization blockers are adjacent and non-overlapping when hybridized to the first target oligonucleotide, wherein the first target oligonucleotide does not comprise an entire index sequence or a complement of an entire index sequence; and a second hybridization blocker comprising an oligonucleotide having an oligonucleotide sequence complementary to a second target oligonucleotide included in the template strand or the complementary strand, wherein the second target oligonucleotide does not comprise an entire Y-stem sequence, a complement of an entire Y-stem sequence, an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand.

Item 17. The set of hybridization blockers of item 16, wherein the second hybridization blocker is a second pair of split hybridization blockers comprising a third split oligonucleotide having an oligonucleotide sequence complementary to a portion C of a second target oligonucleotide included in the template strand or the complementary strand and a fourth split oligonucleotide having an oligonucleotide sequence complementary to a portion D of the second target oligonucleotide included in the template strand or the complementary strand, wherein portion C and portion D are adjacent in the second target oligonucleotide, such that the second pair of split hybridization blockers are adjacent and non-overlapping when hybridized to the second target oligonucleotide, wherein the second target oligonucleotide does not comprise an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand.

Item 18. The set of hybridization blockers of item 16 or 17, further comprising at least one, at least two, at least three, at least four, at least five, at least six, or at least seven, additional hybridization blockers.

Item 19. The kit or set of hybridization blockers of any of items 1 to 18, wherein none of the hybridization blockers overlaps with more than 1 to 12 bases of an index.

Item 20. The kit or set of hybridization blockers of any of items 1 to 19, wherein each hybridization blocker a length in the range of 5 to 60 bases in length, and each hybridization blocker comprises 20-90% of Tₘ-increasing nucleotide analogs.

Item 21. A method of massively parallel sequencing, comprising: hybridizing a plurality of nucleic acid sequencing library molecules with a set of hybridization blockers according to any one of items 16 to 20 or a set of hybridization blockers included in a kit according to any one of items 1 to 15.

Item 22. The method of item 21, wherein the hybridizing a plurality of nucleic acid sequencing library molecules with a set of hybridization blockers is performed prior to a hybridization capture step.

Item 23. The method of item 21 or 22, wherein nucleic acid duplexes formed by hybridizing a plurality of nucleic acid sequencing library molecules with a set of hybridization blockers are washed under high stringency conditions.

Item 24. The method of any one of items 21 to 23, further comprising sequencing at least inserts of the plurality of nucleic acid sequencing library molecules.

Item 25. A set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, comprising: a set of hybridization blockers, each hybridization blocker comprising an oligonucleotide having a nucleic acid sequence which is a complement of an oligonucleotide present in the template strand and/or complementary strand of at least some of the plurality of nucleic acid sequencing library molecules, with the proviso that none of the hybridization blockers comprises an oligonucleotide having a nucleic acid sequence which is a complement of the insert sequence, or an index sequence, if present, of the nucleic acid sequencing library molecules, each oligonucleotide of each hybridization blocker comprising a plurality of Tₘ increasing nucleotide analogs, wherein the set of hybridization blockers comprises at least two pairs of split hybridization blockers, each pair of split hybridization blockers comprising a first split oligonucleotide having an oligonucleotide sequence complementary to a portion A of a target oligonucleotide included in the template strand or the complementary strand and a second split oligonucleotide having an oligonucleotide sequence complementary to a portion B of the target oligonucleotide included in the template strand or the complementary strand, wherein portion A and portion B are adjacent in the target oligonucleotide, and wherein the portion A and the portion B of the target oligonucleotide to which each pair of split hybridization blockers hybridizes, is different.

Item 26. The set of hybridization blockers of item 25, wherein the first adapter comprises a first extension primer-binding site and a first sequencing primer-binding site, the second adapter comprises a second extension primer-binding site and a second sequencing primer-binding site, wherein the complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the first pair of split hybridization blockers comprises a first split oligonucleotide having an oligonucleotide sequence complementary to a portion A of the first sequencing primer-binding site and a second split oligonucleotide having an oligonucleotide sequence complementary to a portion B of the first sequencing primer-binding site, or wherein the first pair of split hybridization blockers comprises a first split oligonucleotide having an oligonucleotide sequence complementary to a portion A of the complement of the first sequencing primer-binding site and a second split oligonucleotide having an oligonucleotide sequence complementary to a portion B of the complement of the first sequencing primer-binding site.

Item 27. The set of hybridization blockers of item 25 or 26, wherein the first adapter comprises a first extension primer-binding site and a first sequencing primer-binding site, the second adapter comprises a second extension primer-binding site and a second sequencing primer-binding site, wherein the complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the second pair of split hybridization blockers comprises a first split oligonucleotide having an oligonucleotide sequence complementary to a portion A of the second sequencing primer-binding site and a second split oligonucleotide having an oligonucleotide sequence complementary to a portion B of the second sequencing primer-binding site, or wherein the second pair of split hybridization blockers comprises a first split oligonucleotide having an oligonucleotide sequence complementary to a portion A of the complement of the second sequencing primer-binding site and a second split oligonucleotide having an oligonucleotide sequence complementary to a portion B of the complement of the second sequencing primer-binding site.

Item 28. The set of hybridization blockers of item 25, wherein the insert of the template strand is a transposon insert disposed between a first transposon end and a second transposon end, the first transposon end adjacent the first sequencing primer-binding site and the second transposon end adjacent the second sequencing primer-binding site, the complementary strand comprising a complement of the transposon insert, the first transposon end and the second transposon end.

Item 29. The set of hybridization blockers of item 28, wherein the first adapter comprises a first extension primer-binding site and a first sequencing primer-binding site, the second adapter comprises a second extension primer-binding site and a second sequencing primer-binding site, wherein complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the first pair of split hybridization blockers comprises a first split oligonucleotide having an oligonucleotide sequence complementary to at least a portion of the first sequencing primer-binding site and a second split oligonucleotide having an oligonucleotide sequence complementary to at least a portion of the first transposon end, or wherein the first pair of split hybridization blockers comprises a first split oligonucleotide having an oligonucleotide sequence complementary to at least a portion of the complement of the first sequencing primer-binding site and a second split oligonucleotide having an oligonucleotide sequence complementary to at least a portion of the complement of the first transposon end.

Item 30. The set of hybridization blockers of item 28 or 29, wherein the first adapter comprises a first extension primer-binding site and a first sequencing primer-binding site, the second adapter comprises a second extension primer-binding site and a second sequencing primer-binding site, wherein complement of the first adapter comprises a complement of the first extension primer-binding site and a complement of the first sequencing primer-binding site, and the complement of the second adapter comprises a complement of the second extension primer-binding site and a complement of the second sequencing primer-binding site, wherein the second pair of split hybridization blockers comprises a first split oligonucleotide having an oligonucleotide sequence complementary to at least a portion of the second sequencing primer-binding site and a second split oligonucleotide having an oligonucleotide sequence complementary to at least a portion of the second transposon end, or wherein the second pair of split hybridization blockers comprises a first split oligonucleotide having an oligonucleotide sequence complementary to at least a portion of the complement of the second sequencing primer-binding site and a second split oligonucleotide having an oligonucleotide sequence complementary to at least a portion of the complement of the second transposon end.

Item 31. The set of hybridization blockers according to any one of items 25 to 30, wherein the hybridization blocker set further comprises an oligonucleotide hybridization blocker comprising a nucleic acid sequence which is a complement of the first extension primer or an oligonucleotide hybridization blocker comprising the nucleic acid sequence of the first extension primer.

Item 32. The set of hybridization blockers according to any one of items 25 to 31, wherein the hybridization blocker set further comprises an oligonucleotide hybridization blocker comprising a nucleic acid sequence which is a complement of the second extension primer or an oligonucleotide hybridization blocker comprising the nucleic acid sequence of the second extension primer.

Item 33. The set of hybridization blockers according to any one of items 25 to 32, for use in massively parallel sequencing of a plurality of nucleic acid sequencing library molecules, wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers: a first hybridization blocker comprising an oligonucleotide sequence which comprises at least a portion of a complement of the first extension primer-binding site; a second hybridization blocker comprising at least a portion of the oligonucleotide sequence of the first sequencing primer-binding site; a third hybridization blocker comprising at least a portion of the oligonucleotide sequence of the first transposon end; a fourth hybridization blocker comprising an oligonucleotide sequence which is a complement of at least a portion of the second sequencing primer-binding site; a fifth hybridization blocker comprising at least a portion of the oligonucleotide sequence of the second extension primer-binding site; a sixth hybridization blocker comprising at least a portion of the oligonucleotide sequence of portion A of the third sequencing primer-binding site; a seventh hybridization blocker comprising the oligonucleotide sequence of portion B of the third sequencing primer-binding site; an eighth hybridization blocker comprising an oligonucleotide which is the complement of portion A of the fourth sequencing primer-binding site; and a ninth hybridization blocker comprising an oligonucleotide which is the complement of portion B of the fourth sequencing primer-binding site.

Item 34. The set of hybridization blockers according to any one of items 25 to 32, for use in massively parallel sequencing of a plurality of nucleic acid sequencing library molecules, wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers: a first hybridization blocker comprising an oligonucleotide sequence which hybridizes to the complement of the first extension primer-binding site; a second hybridization blocker comprising the oligonucleotide sequence which is the complement of the first sequencing primer-binding site; a third hybridization blocker comprising the oligonucleotide sequence hybridizes to the first transposon end; a fourth hybridization blocker comprising an oligonucleotide sequence which hybridizes to the complement of the second sequencing primer-binding site; a fifth hybridization blocker comprising the oligonucleotide sequence which is the complement of the second extension primer-binding site; a sixth hybridization blocker comprising an oligonucleotide sequence which hybridizes to portion A of the third sequencing primer-binding site; a seventh hybridization blocker comprising an oligonucleotide sequence which hybridizes to portion B of the third sequencing primer-binding site; an eighth hybridization blocker comprising an oligonucleotide which hybridizes to the complement of portion A of the fourth sequencing primer-binding site; and a ninth hybridization blocker comprising an oligonucleotide which hybridizes to the complement of portion B of the fourth sequencing primer-binding site.

Item 35. A set of hybridization blockers for use in massively parallel sequencing of a plurality of nucleic acid sequence library inserts of at least four types of nucleic acid sequencing library molecule, the first type of nucleic acid sequencing library molecule comprising, a nucleic acid transposon insert, the transposon insert disposed between, and bonded to, a first adapter and a second adapter, the first adapter comprising at least a first extension primer-binding oligonucleotide, a first sequencing primer-binding oligonucleotide, and a first transposon end sequence, the second adapter comprising at least a second extension primer-binding oligonucleotide, a second sequencing primer-binding oligonucleotide, and a second transposon end sequence, wherein at least one of the first adapter and the second adapter comprises an index oligonucleotide, the second type of nucleic acid sequencing library molecule comprising a complement nucleic acid sequence of the first type of nucleic acid sequencing library molecule wherein the complement nucleic acid sequence of the first type of nucleic acid sequencing library molecule comprises a complement nucleic acid transposon insert, the complement transposon insert disposed between, and bonded to, a complement first adapter and a complement second adapter, the complement first adapter comprising at least a complement first extension primer-binding oligonucleotide, a complement first sequencing primer-binding oligonucleotide, and a complement first transposon end sequence, the complement second adapter comprising at least a complement second extension primer-binding oligonucleotide, a complement second sequencing primer-binding oligonucleotide, and a complement second transposon end sequence, wherein at least one of the complement first adapter and the complement second adapter comprises a complement index oligonucleotide, the third type of nucleic acid sequencing library molecule comprising a nucleic acid insert, each nucleic acid insert disposed between, and bonded to, a third adapter and a fourth adapter, the third adapter comprising at least a third extension primer-binding oligonucleotide, and a third sequencing primer-binding oligonucleotide, the fourth adapter comprising at least a fourth extension primer-binding oligonucleotide, and a fourth sequencing primer-binding oligonucleotide, with the proviso that neither the third adapter nor the fourth adapter comprises a transposon end sequence, wherein at least one of the third adapter and the fourth adapter comprises an index oligonucleotide; the fourth type of nucleic acid sequencing library molecule comprising a complement nucleic acid sequence of the third type of nucleic acid sequencing library molecule wherein the complement nucleic acid sequence of the third type of nucleic acid sequencing library molecule comprises a complement nucleic acid insert, the complement nucleic acid insert disposed between, and bonded to, a complement third adapter and a complement fourth adapter, the complement third adapter comprising at least a complement third extension primer-binding oligonucleotide, and a complement third sequencing primer-binding oligonucleotide, the complement fourth adapter comprising at least a complement fourth extension primer-binding oligonucleotide, and a complement fourth sequencing primer-binding oligonucleotide, with the proviso that neither the complement third adapter nor the complement fourth adapter comprises a transposon end sequence, wherein at least one of the complement third adapter and the complement fourth adapter comprises a complement index oligonucleotide; the set of hybridization blockers comprising at least nine blocking oligonucleotides, wherein each of the at least nine blocking oligonucleotides comprises at least one Tₘ increasing nucleotide, the at least nine blocking oligonucleotides comprising: a first hybridization blocker comprising an oligonucleotide sequence which is the complement of the first extension primer-binding site; a second hybridization blocker comprising the oligonucleotide sequence of the first sequencing primer-binding site; a third hybridization blocker comprising the oligonucleotide sequence of the first transposon end; a fourth hybridization blocker comprising an oligonucleotide sequence which is the complement of the second sequencing primer-binding site; a fifth hybridization blocker comprising the oligonucleotide sequence of the second extension primer-binding site; a sixth hybridization blocker comprising the oligonucleotide sequence of portion A of the third sequencing primer-binding site; a seventh hybridization blocker comprising the oligonucleotide sequence of portion B of the third sequencing primer-binding site; an eighth hybridization blocker comprising an oligonucleotide which is the complement of portion A of the fourth sequencing primer-binding site; and a ninth hybridization blocker comprising an oligonucleotide which is the complement of portion B of the fourth sequencing primer-binding site.

Item 36. The set of hybridization blockers of item 35, for use in massively parallel sequencing of a plurality of nucleic acid sequence library inserts of at least four types of nucleic acid sequencing library molecule, comprising: a first hybridization blocker comprising an oligonucleotide sequence which hybridizes to the complement of the first extension primer-binding site; a second hybridization blocker comprising the oligonucleotide sequence which is the complement of the first sequencing primer-binding site; a third hybridization blocker comprising the oligonucleotide sequence hybridizes to the first transposon end; a fourth hybridization blocker comprising an oligonucleotide sequence which hybridizes to the complement of the second sequencing primer-binding site; a fifth hybridization blocker comprising the oligonucleotide sequence which is the complement of the second extension primer-binding site; a sixth hybridization blocker comprising an oligonucleotide sequence which hybridizes to portion A of the third sequencing primer-binding site; a seventh hybridization blocker comprising an oligonucleotide sequence which hybridizes to portion B of the third sequencing primer-binding site; an eighth hybridization blocker comprising an oligonucleotide which hybridizes to the complement of portion A of the fourth sequencing primer-binding site; and a ninth hybridization blocker comprising an oligonucleotide which hybridizes to the complement of portion B of the fourth sequencing primer-binding site.

Item 37. A set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the insert having a first end and a second end, the first adapter comprising a first primer-binding site and a first target oligonucleotide, the first adapter optionally comprising a first index disposed between the first primer-binding site and the first target oligonucleotide, the first target oligonucleotide disposed between the first index, if present, and the first end of the insert, or the first target oligonucleotide disposed between the first primer-binding site and the first insert end, the second adapter comprising a second target oligonucleotide and a second primer-binding site, the second adapter optionally comprising a second index disposed between the second target oligonucleotide and the second primer-binding site, the second target oligonucleotide disposed between the second end of the insert and the second index, if present, or the second target oligonucleotide disposed between the second target nucleotide and the second primer-binding site, wherein the template strand comprises at least the first or the second index, the complementary strand being the complement of the template strand, comprising: a set of hybridization blockers, each hybridization blocker comprising an oligonucleotide having a nucleic acid sequence which can hybridize to one of: the first target oligonucleotide, the second target oligonucleotide, the complement of the first target oligonucleotide, or the complement of the second target oligonucleotide, each oligonucleotide of each hybridization blocker comprising a plurality of Tₘ increasing nucleotide analogs, wherein the set of hybridization blockers comprises a first set of at least two hybridization blockers that comprise a first pair of split hybridization blockers, each pair of split hybridization blockers comprising a first split oligonucleotide having an oligonucleotide sequence complementary to one of: a portion A of either the first target oligonucleotide or the complement of portion A in the complementary strand, and a second split oligonucleotide having an oligonucleotide sequence complementary to one of: a portion B of the first target oligonucleotide or the complement of portion B in the complementary strand, wherein the first split oligonucleotide and the second split oligonucleotide each hybridize to either the portion A and the portion B on the template strand, or the complement of the portion A and the complement of the portion B on the complementary strand, and wherein the portion A and the portion B of the first target oligonucleotide are different, wherein the set of hybridization blockers further comprises a second pair of split hybridization blockers, the second pair comprising a first split oligonucleotide having an oligonucleotide sequence complementary to one of: a portion C of either the second target oligonucleotide or the complement of portion C in the complementary strand, and a second split oligonucleotide having an oligonucleotide sequence complementary to one of: a portion D of the second target oligonucleotide or the complement of portion D in the complementary strand, wherein the first split oligonucleotide and the second split oligonucleotide of the second pair each hybridize to either the portion C and the portion D on the template strand, or the complement of the portion C and the complement of the portion D on the complementary strand, and wherein the portion C and the portion D of the second target oligonucleotide are different, and, wherein, if the first pair of split hybridization blockers hybridizes to the portion A and the portion B of the template strand, then the second pair of split hybridization blockers hybridizes to the complement of the portion C and the complement of the portion D of the complementary strand, and, if the first pair of split hybridization blockers hybridizes to the complement of the portion A and the complement of the portion B of the complementary strand, then the second pair of split hybridization blockers hybridizes to the portion C and the portion D of the template strand.

Item 38. The set of hybridization blockers of item 37, wherein the portion A and the portion B of the first target oligonucleotide are adjacent.

Item 39. The set of hybridization blockers of item 37 or 38, wherein the portion C and the portion D of the second target oligonucleotide are adjacent.

Item 40. A set of hybridization blockers, each of the hybridization blockers comprising at least one Tₘ increasing nucleotide, the set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, the set of hybridization blockers comprising: a first pair of split hybridization blockers, comprising a first split oligonucleotide having an oligonucleotide sequence complementary to a portion A of a first target oligonucleotide included in the template strand or the complementary strand and a second split oligonucleotide having an oligonucleotide sequence complementary to a portion B of the first target oligonucleotide included in the template strand or the complementary strand, wherein portion A and portion B are adjacent in the target oligonucleotide, such that the first pair of split hybridization blockers are adjacent and non-overlapping when hybridized to the first target oligonucleotide, wherein the first target oligonucleotide does not comprise an entire index sequence or a complement of an entire index sequence; and a second hybridization blocker comprising an oligonucleotide having an oligonucleotide sequence complementary to a second target oligonucleotide included in the template strand or the complementary strand, wherein the second target oligonucleotide does not comprise an entire Y-stem sequence, a complement of an entire Y-stem sequence, an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand.

Item 41. The set of hybridization blockers of item 40, wherein the second hybridization blocker is a second pair of split hybridization blockers comprising a third split oligonucleotide having an oligonucleotide sequence complementary to a portion C of a second target oligonucleotide included in the template strand or the complementary strand and a fourth split oligonucleotide having an oligonucleotide sequence complementary to a portion D of the second target oligonucleotide included in the template strand or the complementary strand, wherein portion C and portion D are adjacent in the second target oligonucleotide, such that the second pair of split hybridization blockers are adjacent and non-overlapping when hybridized to the second target oligonucleotide, wherein the second target oligonucleotide does not comprise an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand.

Item 42. The set of hybridization blockers of item 40 or 41, further comprising at least one, at least two, at least three, at least four, at least five, at least six, or at least seven, additional hybridization blockers.

Item 43. The set of hybridization blockers of any one of items 25 to 42, wherein none of the hybridization blockers overlaps with more than 1 to 12 bases of an index.

Item 44. The set of hybridization blockers of any one of items 25 to 43, wherein each hybridization blocker a length in the range of 5 to 60 bases in length, and each hybridization blocker comprises 20-90% of Tₘ-increasing nucleotide analogs.

Item 45. A kit comprising a set of hybridization blockers according to any one of items 25 to 44.

Item 46. A method of massively parallel sequencing, comprising: hybridizing a plurality of nucleic acid sequencing library molecules with a set of hybridization blockers according to any one of items 25 to 44.

Item 47. The method of item 46, wherein the hybridizing a plurality of nucleic acid sequencing library molecules with a set of hybridization blockers is performed prior to a hybridization capture step.

Item 48. The method of item 46 or 47, wherein nucleic acid duplexes formed by hybridizing a plurality of nucleic acid sequencing library molecules with a set of hybridization blockers are washed under high stringency conditions.

Item 49. The method of any one of items 46 to 48, further comprising sequencing at least inserts of the plurality of nucleic acid sequencing library molecules.

Item 50. A method of massively parallel sequencing comprising hybridizing a plurality of nucleic acid sequencing library molecules with hybridization blockers substantially described and/or shown herein.

Item 51. A set of hybridization blockers substantially as described and/or shown herein.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, comprising:
a set of hybridization blockers, each hybridization blocker comprising an oligonucleotide having a nucleic acid sequence which is a complement of an oligonucleotide present in the template strand and/or complementary strand of at least some of the plurality of nucleic acid sequencing library molecules, with the proviso that none of the hybridization blockers comprises an oligonucleotide having a nucleic acid sequence which is a complement of the insert sequence, or an index sequence, if present, of the nucleic acid sequencing library molecules, each oligonucleotide of each hybridization blocker comprising a plurality of Tₘ increasing nucleotide analogs,
wherein the set of hybridization blockers comprises at least two pairs of split hybridization blockers, each pair of split hybridization blockers comprising a first split oligonucleotide having an oligonucleotide sequence complementary to a portion A of a target oligonucleotide included in the template strand or the complementary strand and a second split oligonucleotide having an oligonucleotide sequence complementary to a portion B of the target oligonucleotide included in the template strand or the complementary strand, wherein portion A and portion B are adjacent in the target oligonucleotide, and wherein the portion A and the portion B of the target oligonucleotide to which each pair of split hybridization blockers hybridizes, is different.

2. The set of hybridization blockers of claim 1, wherein the first adapter comprises a first extension primer-binding site and a first sequencing primer-binding site, the second adapter comprises a second extension primer-binding site and a second sequencing primer-binding site,
wherein the complement of the first adapter comprises a complement of the first extension primer-binding site and a complement of the first sequencing primer-binding site, and the complement of the second adapter comprises a complement of the second extension primer-binding site and a complement of the second sequencing primer-binding site,
wherein the first pair of split hybridization blockers comprises a first split oligonucleotide having an oligonucleotide sequence complementary to a portion A of the first sequencing primer-binding site and a second split oligonucleotide having an oligonucleotide sequence complementary to a portion B of the first sequencing primer-binding site, or wherein the first pair of split hybridization blockers comprises a first split oligonucleotide having an oligonucleotide sequence complementary to a portion A of the complement of the first sequencing primer-binding site and a second split oligonucleotide having an oligonucleotide sequence complementary to a portion B of the complement of the first sequencing primer-binding site.

3. The set of hybridization blockers of claim 1, wherein the first adapter comprises a first extension primer-binding site and a first sequencing primer-binding site, the second adapter comprises a second extension primer-binding site and a second sequencing primer-binding site,
wherein the complement of the first adapter comprises a complement of the first extension primer-binding site and a complement of the first sequencing primer-binding site, and the complement of the second adapter comprises a complement of the second extension primer-binding site and a complement of the second sequencing primer-binding site,
wherein the second pair of split hybridization blockers comprises a first split oligonucleotide having an oligonucleotide sequence complementary to a portion A of the second sequencing primer-binding site and a second split oligonucleotide having an oligonucleotide sequence complementary to a portion B of the second sequencing primer-binding site, or wherein the second pair of split hybridization blockers comprises a first split oligonucleotide having an oligonucleotide sequence complementary to a portion A of the complement of the second sequencing primer-binding site and a second split oligonucleotide having an oligonucleotide sequence complementary to a portion B of the complement of the second sequencing primer-binding site.

4. The set of hybridization blockers of claim 1, wherein the insert of the template strand is a transposon insert disposed between a first transposon end and a second transposon end, the first transposon end adjacent the first sequencing primer-binding site and the second transposon end adjacent the second sequencing primer-binding site, the complementary strand comprising a complement of the transposon insert, the first transposon end and the second transposon end.

5. The set of hybridization blockers of claim 4, wherein the first adapter comprises a first extension primer-binding site and a first sequencing primer-binding site, the second adapter comprises a second extension primer-binding site and a second sequencing primer-binding site,
wherein complement of the first adapter comprises a complement of the first extension primer-binding site and a complement of the first sequencing primer-binding site, and the complement of the second adapter comprises a complement of the second extension primer-binding site and a complement of the second sequencing primer-binding site,
wherein the first pair of split hybridization blockers comprises a first split oligonucleotide having an oligonucleotide sequence complementary to at least a portion of the first sequencing primer-binding site and a second split oligonucleotide having an oligonucleotide sequence complementary to at least a portion of the first transposon end, or wherein the first pair of split hybridization blockers comprises a first split oligonucleotide having an oligonucleotide sequence complementary to at least a portion of the complement of the first sequencing primer-binding site and a second split oligonucleotide having an oligonucleotide sequence complementary to at least a portion of the complement of the first transposon end.

6. The set of hybridization blockers of claim 4, wherein the first adapter comprises a first extension primer-binding site and a first sequencing primer-binding site, the second adapter comprises a second extension primer-binding site and a second sequencing primer-binding site,
wherein complement of the first adapter comprises a complement of the first extension primer-binding site and a complement of the first sequencing primer-binding site, and the complement of the second adapter comprises a complement of the second extension primer-binding site and a complement of the second sequencing primer-binding site,
wherein the second pair of split hybridization blockers comprises a first split oligonucleotide having an oligonucleotide sequence complementary to at least a portion of the second sequencing primer-binding site and a second split oligonucleotide having an oligonucleotide sequence complementary to at least a portion of the second transposon end, or wherein the second pair of split hybridization blockers comprises a first split oligonucleotide having an oligonucleotide sequence complementary to at least a portion of the complement of the second sequencing primer-binding site and a second split oligonucleotide having an oligonucleotide sequence complementary to at least a portion of the complement of the second transposon end.

7. The set of hybridization blockers of claim 1, wherein the hybridization blocker set further comprises an oligonucleotide hybridization blocker comprising a nucleic acid sequence which is a complement of the first extension primer or an oligonucleotide hybridization blocker comprising the nucleic acid sequence of the first extension primer.

8. The set of hybridization blockers of claim 1, wherein the hybridization blocker set further comprises an oligonucleotide hybridization blocker comprising a nucleic acid sequence which is a complement of the second extension primer or an oligonucleotide hybridization blocker comprising the nucleic acid sequence of the second extension primer.

9. The set of hybridization blockers of claim 1, for use in massively parallel sequencing of a plurality of nucleic acid sequencing library molecules, wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers:
a first hybridization blocker comprising an oligonucleotide sequence which comprises at least a portion of a complement of the first extension primer-binding site;
a second hybridization blocker comprising at least a portion of the oligonucleotide sequence of the first sequencing primer-binding site;
a third hybridization blocker comprising at least a portion of the oligonucleotide sequence of the first transposon end;
a fourth hybridization blocker comprising an oligonucleotide sequence which is a complement of at least a portion of the second sequencing primer-binding site;
a fifth hybridization blocker comprising at least a portion of the oligonucleotide sequence of the second extension primer-binding site;
a sixth hybridization blocker comprising at least a portion of the oligonucleotide sequence of portion A of the third sequencing primer-binding site;
a seventh hybridization blocker comprising the oligonucleotide sequence of portion B of the third sequencing primer-binding site;
an eighth hybridization blocker comprising an oligonucleotide which is the complement of portion A of the fourth sequencing primer-binding site; and
a ninth hybridization blocker comprising an oligonucleotide which is the complement of portion B of the fourth sequencing primer-binding site.

10. The set of hybridization blockers of claim 1, for use in massively parallel sequencing of a plurality of nucleic acid sequencing library molecules, wherein at least a first portion of the plurality comprise the insert disposed between and directly adjacent the first adapter and the second adapter and a second portion of the plurality comprise a transposon insert disposed between and directly adjacent a first transposon end and a second transposon end, the first transposon end adjacent the first sequencing primer-binding site of the first adapter and the second transposon end adjacent the second sequencing primer-binding site of the second adapter, and wherein the hybridization blocker set comprises at least nine hybridization blockers:
a first hybridization blocker comprising an oligonucleotide sequence which hybridizes to the complement of the first extension primer-binding site;
a second hybridization blocker comprising the oligonucleotide sequence which is the complement of the first sequencing primer-binding site;
a third hybridization blocker comprising the oligonucleotide sequence hybridizes to the first transposon end;
a fourth hybridization blocker comprising an oligonucleotide sequence which hybridizes to the complement of the second sequencing primer-binding site;
a fifth hybridization blocker comprising the oligonucleotide sequence which is the complement of the second extension primer-binding site;
a sixth hybridization blocker comprising an oligonucleotide sequence which hybridizes to portion A of the third sequencing primer-binding site;
a seventh hybridization blocker comprising an oligonucleotide sequence which hybridizes to portion B of the third sequencing primer-binding site;
an eighth hybridization blocker comprising an oligonucleotide which hybridizes to the complement of portion A of the fourth sequencing primer-binding site; and
a ninth hybridization blocker comprising an oligonucleotide which hybridizes to the complement of portion B of the fourth sequencing primer-binding site.

11. A set of hybridization blockers for use in massively parallel sequencing of a plurality of nucleic acid sequence library inserts of at least four types of nucleic acid sequencing library molecule,
the first type of nucleic acid sequencing library molecule comprising, a nucleic acid transposon insert, the transposon insert disposed between, and bonded to, a first adapter and a second adapter, the first adapter comprising at least a first extension primer-binding oligonucleotide, a first sequencing primer-binding oligonucleotide, and a first transposon end sequence, the second adapter comprising at least a second extension primer-binding oligonucleotide, a second sequencing primer-binding oligonucleotide, and a second transposon end sequence, wherein at least one of the first adapter and the second adapter comprises an index oligonucleotide,
the second type of nucleic acid sequencing library molecule comprising a complement nucleic acid sequence of the first type of nucleic acid sequencing library molecule wherein the complement nucleic acid sequence of the first type of nucleic acid sequencing library molecule comprises a complement nucleic acid transposon insert, the complement transposon insert disposed between, and bonded to, a complement first adapter and a complement second adapter, the complement first adapter comprising at least a complement first extension primer-binding oligonucleotide, a complement first sequencing primer-binding oligonucleotide, and a complement first transposon end sequence, the complement second adapter comprising at least a complement second extension primer-binding oligonucleotide, a complement second sequencing primer-binding oligonucleotide, and a complement second transposon end sequence, wherein at least one of the complement first adapter and the complement second adapter comprises a complement index oligonucleotide,
the third type of nucleic acid sequencing library molecule comprising a nucleic acid insert, each nucleic acid insert disposed between, and bonded to, a third adapter and a fourth adapter, the third adapter comprising at least a third extension primer-binding oligonucleotide, and a third sequencing primer-binding oligonucleotide, the fourth adapter comprising at least a fourth extension primer-binding oligonucleotide, and a fourth sequencing primer-binding oligonucleotide, with the proviso that neither the third adapter nor the fourth adapter comprises a transposon end sequence, wherein at least one of the third adapter and the fourth adapter comprises an index oligonucleotide;
the fourth type of nucleic acid sequencing library molecule comprising a complement nucleic acid sequence of the third type of nucleic acid sequencing library molecule wherein the complement nucleic acid sequence of the third type of nucleic acid sequencing library molecule comprises a complement nucleic acid insert, the complement nucleic acid insert disposed between, and bonded to, a complement third adapter and a complement fourth adapter, the complement third adapter comprising at least a complement third extension primer-binding oligonucleotide, and a complement third sequencing primer-binding oligonucleotide, the complement fourth adapter comprising at least a complement fourth extension primer-binding oligonucleotide, and a complement fourth sequencing primer-binding oligonucleotide, with the proviso that neither the complement third adapter nor the complement fourth adapter comprises a transposon end sequence, wherein at least one of the complement third adapter and the complement fourth adapter comprises a complement index oligonucleotide;
the set of hybridization blockers comprising at least nine blocking oligonucleotides, wherein each of the at least nine blocking oligonucleotides comprises at least one Tₘ increasing nucleotide, the at least nine blocking oligonucleotides comprising:
a first hybridization blocker comprising an oligonucleotide sequence which is the complement of the first extension primer-binding site;
a second hybridization blocker comprising the oligonucleotide sequence of the first sequencing primer-binding site;
a third hybridization blocker comprising the oligonucleotide sequence of the first transposon end;
a fourth hybridization blocker comprising an oligonucleotide sequence which is the complement of the second sequencing primer-binding site;
a fifth hybridization blocker comprising the oligonucleotide sequence of the second extension primer-binding site;
a sixth hybridization blocker comprising the oligonucleotide sequence of portion A of the third sequencing primer-binding site;
a seventh hybridization blocker comprising the oligonucleotide sequence of portion B of the third sequencing primer-binding site;
an eighth hybridization blocker comprising an oligonucleotide which is the complement of portion A of the fourth sequencing primer-binding site; and
a ninth hybridization blocker comprising an oligonucleotide which is the complement of portion B of the fourth sequencing primer-binding site.

12. The set of hybridization blockers of claim 11, for use in massively parallel sequencing of a plurality of nucleic acid sequence library inserts of at least four types of nucleic acid sequencing library molecule, comprising:
a first hybridization blocker comprising an oligonucleotide sequence which hybridizes to the complement of the first extension primer-binding site;
a second hybridization blocker comprising the oligonucleotide sequence which is the complement of the first sequencing primer-binding site;
a third hybridization blocker comprising the oligonucleotide sequence hybridizes to the first transposon end;
a fourth hybridization blocker comprising an oligonucleotide sequence which hybridizes to the complement of the second sequencing primer-binding site;
a fifth hybridization blocker comprising the oligonucleotide sequence which is the complement of the second extension primer-binding site;
a sixth hybridization blocker comprising an oligonucleotide sequence which hybridizes to portion A of the third sequencing primer-binding site;
a seventh hybridization blocker comprising an oligonucleotide sequence which hybridizes to portion B of the third sequencing primer-binding site;
an eighth hybridization blocker comprising an oligonucleotide which hybridizes to the complement of portion A of the fourth sequencing primer-binding site; and
a ninth hybridization blocker comprising an oligonucleotide which hybridizes to the complement of portion B of the fourth sequencing primer-binding site.

13. A set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the insert having a first end and a second end, the first adapter comprising a first primer-binding site and a first target oligonucleotide, the first adapter optionally comprising a first index disposed between the first primer-binding site and the first target oligonucleotide, the first target oligonucleotide disposed between the first index, if present, and the first end of the insert, or the first target oligonucleotide disposed between the first primer-binding site and the first insert end, the second adapter comprising a second target oligonucleotide and a second primer-binding site, the second adapter optionally comprising a second index disposed between the second target oligonucleotide and the second primer-binding site, the second target oligonucleotide disposed between the second end of the insert and the second index, if present, or the second target oligonucleotide disposed between the second target nucleotide and the second primer-binding site, wherein the template strand comprises at least the first or the second index, the complementary strand being the complement of the template strand, the kit comprising:
a set of hybridization blockers, each hybridization blocker comprising an oligonucleotide having a nucleic acid sequence which can hybridize to one of: the first target oligonucleotide, the second target oligonucleotide, the complement of the first target oligonucleotide, or the complement of the second target oligonucleotide, each oligonucleotide of each hybridization blocker comprising a plurality of Tₘ increasing nucleotide analogs,
wherein the set of hybridization blockers comprises a first set of at least two hybridization blockers that comprise a first pair of split hybridization blockers, each pair of split hybridization blockers comprising a first split oligonucleotide having an oligonucleotide sequence complementary to one of: a portion A of either the first target oligonucleotide or the complement of portion A in the complementary strand, and a second split oligonucleotide having an oligonucleotide sequence complementary to one of: a portion B of the first target oligonucleotide or the complement of portion B in the complementary strand, wherein the first split oligonucleotide and the second split oligonucleotide each hybridize to either the portion A and the portion B on the template strand, or the complement of the portion A and the complement of the portion B on the complementary strand, and wherein the portion A and the portion B of the first target oligonucleotide are different,
wherein the set of hybridization blockers further comprises a second pair of split hybridization blockers, the second pair comprising a first split oligonucleotide having an oligonucleotide sequence complementary to one of: a portion C of either the second target oligonucleotide or the complement of portion C in the complementary strand, and a second split oligonucleotide having an oligonucleotide sequence complementary to one of: a portion D of the second target oligonucleotide or the complement of portion D in the complementary strand, wherein the first split oligonucleotide and the second split oligonucleotide of the second pair each hybridize to either the portion C and the portion D on the template strand, or the complement of the portion C and the complement of the portion D on the complementary strand, and wherein the portion C and the portion D of the second target oligonucleotide are different, and,
wherein, if the first pair of split hybridization blockers hybridizes to the portion A and the portion B of the template strand, then the second pair of split hybridization blockers hybridizes to the complement of the portion C and the complement of the portion D of the complementary strand, and, if the first pair of split hybridization blockers hybridizes to the complement of the portion A and the complement of the portion B of the complementary strand, then the second pair of split hybridization blockers hybridizes to the portion C and the portion D of the template strand.

14. The set of hybridization blockers of claim 13, wherein the portion A and the portion B of the first target oligonucleotide are adjacent.

15. The set of hybridization blockers of claim 13, wherein the portion C and the portion D of the second target oligonucleotide are adjacent.

16. A set of hybridization blockers, each of the hybridization blockers comprising at least one Tm increasing nucleotide, the set of hybridization blockers for use in massively parallel sequencing of a plurality of nucleic acid sequencing library molecules, the plurality of nucleic acid sequencing library molecules each having a template strand and a complementary strand, the template strand comprising an insert disposed between a first adapter and a second adapter, the complementary strand comprising a complement of the insert disposed between a complement of the first adapter and a complement of the second adapter, the set of hybridization blockers comprising:
a first pair of split hybridization blockers, comprising a first split oligonucleotide having an oligonucleotide sequence complementary to a portion A of a first target oligonucleotide included in the template strand or the complementary strand and a second split oligonucleotide having an oligonucleotide sequence complementary to a portion B of the first target oligonucleotide included in the template strand or the complementary strand, wherein portion A and portion B are adjacent in the target oligonucleotide, such that the first pair of split hybridization blockers are adjacent and non-overlapping when hybridized to the first target oligonucleotide, wherein the first target oligonucleotide does not comprise an entire index sequence or a complement of an entire index sequence; and
a second hybridization blocker comprising an oligonucleotide having an oligonucleotide sequence complementary to a second target oligonucleotide included in the template strand or the complementary strand, wherein the second target oligonucleotide does not comprise an entire Y-stem sequence, a complement of an entire Y-stem sequence, an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand.

17. The set of hybridization blockers of claim 16, wherein the second hybridization blocker is a second pair of split hybridization blockers comprising a third split oligonucleotide having an oligonucleotide sequence complementary to a portion C of a second target oligonucleotide included in the template strand or the complementary strand and a fourth split oligonucleotide having an oligonucleotide sequence complementary to a portion D of the second target oligonucleotide included in the template strand or the complementary strand, wherein portion C and portion D are adjacent in the second target oligonucleotide, such that the second pair of split hybridization blockers are adjacent and non-overlapping when hybridized to the second target oligonucleotide, wherein the second target oligonucleotide does not comprise an entire index sequence or a complement of an entire index sequence, and wherein, when the first target of the first pair of split hybridization blockers is in the template strand, the second target oligonucleotide of the second hybridization blocker is in the complementary strand, or, when the first target of the first pair of split hybridization blockers is in the complementary strand, the second target oligonucleotide of the second hybridization blocker is in the template strand.

18. The set of hybridization blockers of claim 16, further comprising at least one, at least two, at least three, at least four, at least five, at least six, or at least seven, additional hybridization blockers.

19. The set of hybridization blockers of claim 1, wherein none of the hybridization blockers overlaps with more than 1 to 12 bases of an index.

20. The set of hybridization blockers according to claim 1, wherein each hybridization blocker a length in the range of 5 to 60 bases in length, and each hybridization blocker comprises 20-90% of Tₘ-increasing nucleotide analogs.

21. A kit comprising a set of hybridization blockers according to claim 1.

22. A method of massively parallel sequencing, comprising: hybridizing a plurality of nucleic acid sequencing library molecules with a set of hybridization blockers according to claim 1.

23. The method of claim 22, wherein the hybridizing a plurality of nucleic acid sequencing library molecules with a set of hybridization blockers is performed prior to a hybridization capture step.

24. The method of claim 22, wherein nucleic acid duplexes formed by hybridizing a plurality of nucleic acid sequencing library molecules with a set of hybridization blockers are washed under high stringency conditions.

25. The method of claim 22, further comprising sequencing at least inserts of the plurality of nucleic acid sequencing library molecules.


SEQUENCE LISTING (28 sequences)

SEQ ID NO: 1 - DNA, 24 bp, Artificial Sequence
  Hybridization blocker
  misc_feature (3)..(3): locked nucleic acid monomer
  misc_feature (5)..(10): locked nucleic acid monomer
  misc_feature (12)..(15): locked nucleic acid monomer
  misc_feature (17)..(23): locked nucleic acid monomer
caagcagaagacggcatacgagat

SEQ ID NO: 2 - DNA, 24 bp, Artificial Sequence
  Hybridization blocker
  misc_feature (2)..(2): locked nucleic acid monomer
  misc_feature (3)..(3): locked nucleic acid monomer
  misc_feature (5)..(8): locked nucleic acid monomer
  misc_feature (11)..(15): locked nucleic acid monomer
  misc_feature (17)..(21): locked nucleic acid monomer
  misc_feature (23)..(24): locked nucleic acid monomer
atctcgtatgccgtcttctgcttg

SEQ ID NO: 3 - DNA, 33 bp, Artificial Sequence
  Hybridization blocker
  misc_feature (3)..(3): locked nucleic acid monomer
  misc_feature (5)..(5): locked nucleic acid monomer
  misc_feature (7)..(8): locked nucleic acid monomer
  misc_feature (11)..(13): locked nucleic acid monomer
  misc_feature (15)..(15): locked nucleic acid monomer
  misc_feature (17)..(17): locked nucleic acid monomer
  misc_feature (19)..(19): locked nucleic acid monomer
  misc_feature (21)..(21): locked nucleic acid monomer
  misc_feature (23)..(23): locked nucleic acid monomer
  misc_feature (25)..(25): locked nucleic acid monomer
  misc_feature (27)..(30): locked nucleic acid monomer
  misc_feature (32)..(32): locked nucleic acid monomer
gtgactggagttcagacgtgtgctcttccgatc

SEQ ID NO: 4 - DNA, 33 bp, Artificial Sequence
  Hybridization blocker
  misc_feature (3)..(6): locked nucleic acid monomer
  misc_feature (8)..(9): locked nucleic acid monomer
  misc_feature (12)..(12): locked nucleic acid monomer
  misc_feature (14)..(14): locked nucleic acid monomer
  misc_feature (16)..(16): locked nucleic acid monomer
  misc_feature (19)..(19): locked nucleic acid monomer
  misc_feature (21)..(21): locked nucleic acid monomer
  misc_feature (24)..(24): locked nucleic acid monomer
  misc_feature (26)..(27): locked nucleic acid monomer
  misc_feature (29)..(29): locked nucleic acid monomer
  misc_feature (31)..(31): locked nucleic acid monomer
  misc_feature (33)..(33): locked nucleic acid monomer
gatcggaagagcacacgtctgaactccagtcac

SEQ ID NO: 5 - DNA, 29 bp, Artificial Sequence
  Hybridization blocker
  misc_feature (3)..(3): locked nucleic acid monomer
  misc_feature (4)..(4): locked nucleic acid monomer
  misc_feature (6)..(6): locked nucleic acid monomer
  misc_feature (8)..(8): locked nucleic acid monomer
  misc_feature (11)..(12): locked nucleic acid monomer
  misc_feature (14)..(15): locked nucleic acid monomer
  misc_feature (17)..(18): locked nucleic acid monomer
  misc_feature (21)..(21): locked nucleic acid monomer
  misc_feature (24)..(24): locked nucleic acid monomer
  misc_feature (27)..(28): locked nucleic acid monomer
aatgatacggcgaccaccgagatctacac

SEQ ID NO: 6 - DNA, 29 bp, Artificial Sequence
  Hybridization blocker
  misc_feature (2)..(3): locked nucleic acid
  misc_feature (5)..(6): locked nucleic acid monomer
  misc_feature (8)..(9): locked nucleic acid monomer
  misc_feature (11)..(11): locked nucleic acid monomer
  misc_feature (13)..(13): locked nucleic acid monomer
  misc_feature (15)..(15): locked nucleic acid monomer
  misc_feature (17)..(18): locked nucleic acid monomer
  misc_feature (20)..(21): locked nucleic acid monomer
  misc_feature (23)..(24): locked nucleic acid monomer
  misc_feature (26)..(26): locked nucleic acid monomer
  misc_feature (28)..(28): locked nucleic acid monomer
gtgtagatctcggtggtcgccgtatcatt

SEQ ID NO: 7 - DNA, 32 bp, Artificial Sequence
  Hybridization blocker
  misc_feature (2)..(2): locked nucleic acid monomer
  misc_feature (4)..(4): locked nucleic acid monomer
  misc_feature (6)..(6): locked nucleic acid monomer
  misc_feature (9)..(10): locked nucleic acid monomer
  misc_feature (12)..(12): locked nucleic acid monomer
  misc_feature (15)..(15): locked nucleic acid monomer
  misc_feature (17)..(18): locked nucleic acid monomer
  misc_feature (20)..(20): locked nucleic acid monomer
  misc_feature (22)..(22): locked nucleic acid monomer
  misc_feature (24)..(24): locked nucleic acid monomer
  misc_feature (27)..(27): locked nucleic acid monomer
  misc_feature (30)..(30): locked nucleic acid monomer
acactctttccctacacgacgctcttccgatc

SEQ ID NO: 8 - DNA, 32 bp, Artificial Sequence
  Hybridization blocker
  misc_feature (3)..(4): locked nucleic acid monomer
  misc_feature (6)..(6): locked nucleic acid monomer
  misc_feature (8)..(9): locked nucleic acid monomer
  misc_feature (12)..(12): locked nucleic acid monomer
  misc_feature (14)..(15): locked nucleic acid monomer
  misc_feature (17)..(17): locked nucleic acid monomer
  misc_feature (19)..(23): locked nucleic acid monomer
  misc_feature (26)..(28): locked nucleic acid monomer
  misc_feature (30)..(31): locked nucleic acid monomer
gatcggaagagcgtcgtgtagggaaagagtgt

SEQ ID NO: 9 - DNA, 33 bp, Artificial Sequence
  Hybridization blocker
  misc_feature (2)..(3): locked nucleic acid monomer
  misc_feature (5)..(5): locked nucleic acid monomer
  misc_feature (7)..(7): locked nucleic acid monomer
  misc_feature (9)..(10): locked nucleic acid monomer
  misc_feature (13)..(13): locked nucleic acid monomer
  misc_feature (15)..(15): locked nucleic acid monomer
  misc_feature (18)..(18): locked nucleic acid monomer
  misc_feature (20)..(20): locked nucleic acid monomer
  misc_feature (22)..(22): locked nucleic acid monomer
  misc_feature (24)..(24): locked nucleic acid monomer
  misc_feature (27)..(27): locked nucleic acid monomer
  misc_feature (29)..(29): locked nucleic acid monomer
  misc_feature (31)..(32): locked nucleic acid monomer
ctgtctcttatacacatctgacgctgccgacga

SEQ ID NO: 10 - DNA, 34 bp, Artificial Sequence
  Hybridization blocker
  misc_feature (2)..(3): locked nucleic acid monomer
  misc_feature (5)..(5): locked nucleic acid monomer
  misc_feature (7)..(7): locked nucleic acid monomer
  misc_feature (9)..(10): locked nucleic acid monomer
  misc_feature (13)..(13): locked nucleic acid monomer
  misc_feature (15)..(16): locked nucleic acid monomer
  misc_feature (18)..(18): locked nucleic acid monomer
  misc_feature (20)..(20): locked nucleic acid monomer
  misc_feature (23)..(23): locked nucleic acid monomer
  misc_feature (25)..(25): locked nucleic acid monomer
  misc_feature (27)..(27): locked nucleic acid monomer
  misc_feature (29)..(29): locked nucleic acid monomer
  misc_feature (31)..(32): locked nucleic acid monomer
  misc_feature (34)..(34): locked nucleic acid monomer
ctgtctcttatacacatctccgagcccacgagac

SEQ ID NO: 11 - DNA, 17 bp, Artificial Sequence
  Hybridization blocker
  misc_feature (3)..(3): locked nucleic acid monomer
  misc_feature (5)..(5): locked nucleic acid monomer
  misc_feature (7)..(8): locked nucleic acid monomer
  misc_feature (11)..(13): locked nucleic acid monomer
  misc_feature (15)..(15): locked nucleic acid monomer
  misc_feature (17)..(17): locked nucleic acid monomer
gtgactggagttcagac

SEQ ID NO: 12 - DNA, 16 bp, Artificial Sequence
  Hybridization blocker
  misc_feature (2)..(2): locked nucleic acid monomer
  misc_feature (4)..(4): locked nucleic acid monomer
  misc_feature (6)..(6): locked nucleic acid monomer
  misc_feature (8)..(8): locked nucleic acid monomer
  misc_feature (10)..(13): locked nucleic acid monomer
  misc_feature (15)..(15): locked nucleic acid monomer
gtgtgctcttccgatc

SEQ ID NO: 13 - DNA, 16 bp, Artificial Sequence
  Hybridization blocker
  misc_feature (2)..(2): locked nucleic acid monomer
  misc_feature (4)..(4): locked nucleic acid monomer
  misc_feature (6)..(6): locked nucleic acid monomer
  misc_feature (9)..(10): locked nucleic acid monomer
  misc_feature (12)..(12): locked nucleic acid monomer
  misc_feature (15)..(15): locked nucleic acid monomer
acactctttccctaca

SEQ ID NO: 14 - DNA, 16 bp, Artificial Sequence
  Hybridization blocker
  misc_feature (1)..(1): locked nucleic acid monomer
  misc_feature (2)..(2): locked nucleic acid monomer
  misc_feature (4)..(4): locked nucleic acid monomer
  misc_feature (6)..(6): locked nucleic acid monomer
  misc_feature (8)..(8): locked nucleic acid monomer
  misc_feature (11)..(11): locked nucleic acid monomer
  misc_feature (14)..(14): locked nucleic acid monomer
cgacgctcttccgatc

SEQ ID NO: 15 - DNA, 17 bp, Artificial Sequence
  Hybridization blocker
  misc_feature (2)..(3): locked nucleic acid monomer
  misc_feature (5)..(9): locked nucleic acid monomer
  misc_feature (11)..(15): locked nucleic acid monomer
  misc_feature (17)..(17): locked nucleic acid monomer
gtgactggagttcagac

SEQ ID NO: 16 - DNA, 16 bp, Artificial Sequence
  Hybridization blocker
  misc_feature (2)..(8): locked nucleic acid monomer
  misc_feature (10)..(13): locked nucleic acid monomer
  misc_feature (15)..(15): locked nucleic acid monomer
gtgtgctcttccgatc

SEQ ID NO: 17 - DNA, 16 bp, Artificial Sequence
  Hybridization blocker
  misc_feature (2)..(2): locked nucleic acid monomer
  misc_feature (4)..(6): locked nucleic acid monomer
  misc_feature (8)..(12): locked nucleic acid monomer
  misc_feature (15)..(15): locked nucleic acid monomer
acactctttccctaca

SEQ ID NO: 18 - DNA, 16 bp, Artificial Sequence
  Hybridization blocker
  misc_feature (1)..(2): locked nucleic acid monomer
  misc_feature (4)..(8): locked nucleic acid monomer
  misc_feature (10)..(14): locked nucleic acid monomer
cgacgctcttccgatc

SEQ ID NO: 19 - DNA, 19 bp, Artificial Sequence
  Hybridization blocker
  misc_feature (2)..(3): locked nucleic acid monomer
  misc_feature (5)..(9): locked nucleic acid monomer
  misc_feature (11)..(15): locked nucleic acid monomer
  misc_feature (17)..(18): locked nucleic acid monomer
gtgactggagttcagacgt

SEQ ID NO: 20 - DNA, 18 bp, Artificial Sequence
  Hybridization blocker
  misc_feature (1)..(1): locked nucleic acid monomer
  misc_feature (3)..(6): locked nucleic acid monomer
  misc_feature (8)..(10): locked nucleic acid monomer
  misc_feature (12)..(12): locked nucleic acid monomer
  misc_feature (14)..(14): locked nucleic acid monomer
  misc_feature (16)..(17): locked nucleic acid monomer
gatcggaagagcacacgt

SEQ ID NO: 21 - DNA, 19 bp, Artificial Sequence
  Hybridization blocker
  misc_feature (2)..(2): locked nucleic acid monomer
  misc_feature (4)..(4): locked nucleic acid monomer
  misc_feature (6)..(6): locked nucleic acid monomer
  misc_feature (8)..(13): locked nucleic acid monomer
  misc_feature (15)..(15): locked nucleic acid monomer
  misc_feature (17)..(18): locked nucleic acid monomer
acactctttccctacacga

SEQ ID NO: 22 - DNA, 17 bp, Artificial Sequence
  Hybridization blocker
  misc_feature (1)..(1): locked nucleic acid monomer
  misc_feature (3)..(6): locked nucleic acid monomer
  misc_feature (8)..(10): locked nucleic acid monomer
  misc_feature (12)..(12): locked nucleic acid monomer
  misc_feature (14)..(16): locked nucleic acid monomer
gatcggaagagcgtcgt

SEQ ID NO: 23 - DNA, 17 bp, Artificial Sequence
  Hybridization blocker
  misc_feature (2)..(6): locked nucleic acid monomer
  misc_feature (8)..(8): locked nucleic acid monomer
  misc_feature (10)..(13): locked nucleic acid monomer
  misc_feature (15)..(16): locked nucleic acid monomer
atgtgtataagagacag

SEQ ID NO: 24 - DNA, 16 bp, Artificial Sequence
  Hybridization blocker
  misc_feature (2)..(2): locked nucleic acid monomer
  misc_feature (4)..(5): locked nucleic acid monomer
  misc_feature (8)..(9): locked nucleic acid monomer
  misc_feature (11)..(11): locked nucleic acid monomer
  misc_feature (13)..(15): locked nucleic acid monomer
tcgtcggcagcgtcag

SEQ ID NO: 25 - DNA, 17 bp, Artificial Sequence
  Hybridization blocker
  misc_feature (2)..(3): locked nucleic acid monomer
  misc_feature (5)..(5): locked nucleic acid monomer
  misc_feature (7)..(9): locked nucleic acid monomer
  misc_feature (11)..(11): locked nucleic acid monomer
  misc_feature (13)..(13): locked nucleic acid monomer
  misc_feature (15)..(15): locked nucleic acid monomer
gtctcgtgggctcggag

SEQ ID NO: 26 - DNA, 17 bp, Artificial Sequence
  Hybridization blocker
  misc_feature (2)..(7): locked nucleic acid monomer
  misc_feature (9)..(13): locked nucleic acid monomer
  misc_feature (15)..(16): locked nucleic acid monomer
ctgtctcttatacacat

SEQ ID NO: 27 - DNA, 16 bp, Artificial Sequence
  Hybridization blocker
  misc_feature (2)..(3): locked nucleic acid monomer
  misc_feature (5)..(5): locked nucleic acid monomer
  misc_feature (7)..(7): locked nucleic acid monomer
  misc_feature (10)..(10): locked nucleic acid monomer
  misc_feature (12)..(12): locked nucleic acid monomer
  misc_feature (14)..(15): locked nucleic acid monomer
ctgacgctgccgacga

SEQ ID NO: 28 - DNA, 17 bp, Artificial Sequence
  Hybridization blocker
  misc_feature (3)..(3): locked nucleic acid monomer
  misc_feature (6)..(6): locked nucleic acid monomer
  misc_feature (8)..(8): locked nucleic acid monomer
  misc_feature (10)..(10): locked nucleic acid monomer
  misc_feature (12)..(12): locked nucleic acid monomer
  misc_feature (14)..(15): locked nucleic acid monomer
  misc_feature (17)..(17): locked nucleic acid monomer
ctccgagcccacgagac